(19) United States
(12) Patent Application Publication
(10) Pub. No.: US 2010/0129807 A1
(43) Pub. Date: May 27, 2010

(54) STABILIZATION OF CYCLIC PEPTIDE STRUCTURES

(76) Inventor: Ronald C. Geyer, Saskatoon (CA)

Correspondence Address: KLARQUIST SPARKMAN, LLP, 121 SW SALMON STREET, SUITE 1600, PORTLAND, OR, 97204, US

(21) Appl. No.: 12/522,708

(22) PCT Filed: Jan. 10, 2008
(86) PCT No.: PCT/CA08/00048

§ 371 Date: Sep. 11, 2009

Related U.S. Application Data
(60) Provisional application No. 60/844,279, filed on Sep. 14, 2006.

(51) Int. Cl. C12Q 1/68; C07H 21/00; C12N 5/07; C07K 16/00

(52) U.S. Cl. 435/6; 536/23.1; 536/23.53; 435/348; 530/387.1

(57) ABSTRACT

In various aspects, the invention provides methods for cyclizing proteins, including methods for enhancing the stability of cyclized proteins under cytosolic conditions. The invention also provides various methods for using the cyclized proteins. For example, cyclized proteins of the invention may be used in screening assays analogous to the yeast two hybrid assay. Selected embodiments of the invention provide cyclized single chain variable fragment (ScFv) molecules, including molecules in the form of an immunoglobulin fold.

### BACKGROUND

Advances in functional genomic analyses are providing data sets that enhance our ability to evaluate the therapeutic potential of proteins. Functional genomic data alone, however, produces relatively low quality information on the contribution of any individual protein to a disease or process. To obtain stronger conclusions on the therapeutic potential of a protein, it is necessary to supplement functional genomic data with directed experimentation. A major bottleneck in performing directed experimentation is a lack of high throughput technologies for the reverse analysis of protein function. In reverse analysis, the investigator starts with a gene hypothesized to be associated with a disease or process and uses directed experimentation to validate this association. Within organisms, directed experimentation has traditionally relied on genetic approaches that inactivate genes, either by deleting or creating loss of function mutations in genes. Although genetic approaches are highly informative, they are often difficult to perform on a large scale and in variety of organisms.

Trans dominant agents such as small molecules, antisense RNA, ribozymes, RNAi, antibodies, and dominant negative proteins have been developed that make it easier to perform reverse analysis in diploid organisms (Geyer, C. R. & Brent, R. (2000) Methods Enzymol. 328:178-208). These agents inactivate gene products without altering the genetic material that encodes them. In addition to a dominant mode of action, systematic reverse analysis of protein function requires agents that can be easily and rapidly generated against any given target, that can inhibit protein interactions and activities, and that can block specific interactions with a protein while leaving other interactions unperturbed. To demonstrate the therapeutic potential of inhibiting targets with small molecule drugs, reverse analysis must also be performed with reagents that directly inhibit the target rather than blocking steps in transcription or translation of the target.

Intracellular inhibitors of protein function with these characteristics can be rapidly obtained by genetically selecting conformationally constrained, scaffolded peptides (peptide aptamers) from combinatorial peptide aptamer libraries using the yeast two-hybrid assay (Geyer, C. R. & Brent, R. (2000) Methods Enzymol. 328:178-208). Constrained peptides are preferred as they generally bind tighter and are more stable (Davidson, A. R. & Sauer, R. T. (1994) Proc. Natl. Acad. Sci. USA 91:2146-2150) than linear peptides. Combinatorial libraries of peptide aptamers should in principle contain members that bind any target. The scaffold protein enhances solubility and allows a transcription activation domain to be fused to the peptide aptamer, which is essential for the yeast two-hybrid assay. Peptide aptamers are useful for validating proteins as therapeutic targets however displaying peptides on the surface of scaffolds limits their use as drugs or drug-leads as they are usually not membrane permeable and they are susceptible to degradation by proteases. The size of the scaffold protein also prevents the synthesis of peptide aptamers by synthetic peptide chemistry and makes solving their structure difficult.

Alternatively, peptides can be constrained by cyclization and there are many examples of natural and synthetic cyclic peptide inhibitors (Horswill, A. R. & Benkovic, S. J. (2005) Cell Cycle 4:552-555). Recently, methods have been developed to express genetically encoded cyclic peptides using engineered inteins (Scott, C. P. et al. (1999) Proc. Natl. Acad. Sci. USA 96:13638-13643). Cyclic peptide have advantages over peptide aptamers in that they are resistant to exoproteases and their small size makes them amenable to chemical synthesis, structural studies, and membrane transport. Combinatorial libraries of cyclic peptides have been screened using forward and reverse approaches to isolate cyclic peptides that inhibit cellular processes (Kinsella, T. M. et al. (2002) J. Biol. Chem. 277:37512-37518, Nilsson, L. O. et al. (2005) Protein Pept. Lett. 12:795-799) and disrupt protein interactions (Horswill, A. R. et al. (2004) Proc. Natl. Acad. Sci. USA 101:15591-15596), respectively.

Antibodies are non-cyclic proteins that have a very well characterized structure made up of a number of domains having a recognizable tertiary structure. Each domain in an antibody molecule has a similar structure of two beta sheets packed tightly against each other in a compressed antiparallel beta barrel. This conserved structure is termed the immunoglobulin fold. The fold is generally stabilized by hydrogen bonding between the beta strands of each sheet, by hydrophobic bonding between residues of opposite sheets in the interior, and by a disulfide bond between the sheets. The folds of variable domains have 9 beta strands arranged in two sheets of 4 and 5 strands. Each variable region is made up from three complementarity determining regions (CDR) separated by four framework regions (FR). The CDR's are the most variable part of the variable regions, and perform the antigen binding function. It has been shown that the function of binding antigens can also be performed by fragments of a whole antibody. Example binding fragments are (i) the Fab fragment consisting of the VL, VH, CL and CH1 domains; (ii) the Fd fragment consisting of the VH and CHI domains; (iii) the Fv fragment consisting of the VL and VH domains of a single arm of an antibody, (iv) the dAb fragment (Ward, E. S. et al., Nature 341, 544-546 (1989) which consists of a VH domain; (v) isolated CDR regions; and (vi) F(ab′).sub.2 fragments, a bivalent fragment comprising two Fab fragments linked by a disulphide bridge at the hinge region. Although the two domains of the Fv fragment are coded for by separate genes, it has proved possible to make a synthetic linker that enables them to be made as a single protein chain (known as single chain Fv (scFv); Bird, R. E. et al., Science 242, 423-426 (1988) Huston, J. S. et al., Proc. Natl. Acad. Sci., USA 85, 5879-5883 (1988)) by recombinant methods.

### SUMMARY

One aspect of the invention discloses a genetic assay that may be used to isolate peptide lariats that interact with a target protein using the yeast two-hybrid interaction trap (Gyuris, J. et al. (1993) Cell 75:791-803). A lariat consists of a cyclic peptide or “noose” region with a covalently attached transcription activation domain. The invention provides lariats that are compatible with the yeast two-hybrid system by engineering the intein cyclic peptide producing system (Scott, C. P. et al. (1999) Proc. Natl. Acad. Sci. USA 96:13638-13643) to halt the cyclic peptide reaction at an intermediate step, which produces a lariat that contains a transcription activation domain covalently attached through an amide bond to a lactone-cyclized peptide. Lariat peptides or cyclic peptides based on the noose sequence can be used to study the function or validate the therapeutic potential of protein targets.

In one specific embodiment, the invention exemplifies the feasibility of the foregoing approach by generating inhibitors of the bacterial repressor protein LexA. LexA represents a putative antimicrobial target, which when inhibited should potentiate that activity of cytotoxic antibiotics. When LexA is bound by activated RecA it undergoes autoproteolysis and no longer represses genes in its regulon (Lin, L. L. & Little, J. W. (1988) Bacteriol. 170:2163-2173). LexA mutants that block autoproteolysis (Walker, G. C. (1984) Microbiol. Rev. 48:60-93) make bacteria more sensitive to stress induced by compounds such as the DNA damaging reagent mitomycin C (MMC) (Lin, L. L. & Little, J. W. (1988) Bacteriol. 170:2163-2173) and they decrease antibiotic resistance (Cirz, R. T. et al. (2005) PLoS Biol. 3:e176, Miller, C. et al. (2004) Science 305:1629-1631). LexA inhibitors that block autoproteolysis would increase the sensitivity of bacteria to cytotoxic reagents and since LexA is not present in humans it would have no effect on host DNA damage repair systems.

Various embodiments of the invention make use of vectors comprising a host-operable promoter operably linked to a nucleic acid molecule comprising, in order, an activity domain, a modified C-intein domain, an insert, a modified N-intein domain and a transcription termination sequence.

In some embodiments, there is provided a modified intein lariat library comprising a host-operable promoter operably linked to a nucleic acid molecule comprising, in order, an activity domain, a modified C-intein, an insert having a random peptide or antibody single chain variable fragment (ScFv) encoding oligonucleotides, or a random genomic fragment inserted therein, a modified N-intein and a transcription termination sequence. Here, ScFv refers to an antibody fragment consisting of immunoglobulin variable (V) domains of heavy (H) and light (L) chains held together by a short linker (Tanaka, T. et al. (2003) Nucl. Acids Res. 31:e23). De novo ScFvs can be constructed that contain specific framework regions from chosen light and heavy chain variable domains and that contain random complementary determining regions. Alternatively, immune and non-immune ScFv libraries can be generated using RT-PCR to amplify light and heavy chain variable domains from total RNA purified from B lymphocytes of peripheral blood. The immune libraries can be generated from animals challenged with a specific antigen or from animals with a specific disease. Genomic fragments refer to randomly or rationally generated fragments of DNA derived from genomic DNA or cDNA.

In alternative embodiments, methods are provided for identifying a cyclic-like peptide, ScFv, or genomic fragment that interacts with a target molecule. These methods may for example take place inside of an host organisms comprising: (i) transforming the modified intein library as described above into a suitable host or host cells or cell line; (ii) transforming said with a nucleic acid molecule encoding the target molecule attached to the second activity domain arranged for expression in said host; (iii) identifying host cells comprising a detectable product generated by bringing together the activity domains through an interaction between a member of the intein library and the target molecule; and (iv) recovering the library member from the host cell expressing the detectable product and sequencing the random peptide, ScFv or genomic fragment encoding oligonucleotide. Many assays have been reported for detecting protein interactions within cells including two-hybrid systems (reviewed in Vidal M. & Legrain P. (1999) Nucl. Acid Res. 27:919), split-ubiquitin system (Stagljar et al., (1998) Proc. Natl. Acad. Sci. USA 95:5187), protein-fragment complementation assay (Remy I. & Michnick S. W. (1999) Proc. Natl. Acad. Sci. USA 96:5394), repressor reconstitution assay (Hirst et al., (2001) Proc. Natl. Acad. Sci. USA 98:8726), and SOS recruitment system (Broder et al., (1998) Curr. Biol. 8:1121). Any of these assays or similar assays not listed that detect protein interactions using reporter genes/proteins in cells can be used to isolate cyclic-like peptides, genomic fragments or ScFvs that interact with a protein target. Alternatively, many assays have been reported that couple the DNA encoding a protein to the expressed protein including phage display (Smith, G. P. (1985) Science 228:1315), bacterial display (Francisco, et al., (1993) Proc. Natl. Acad. Sci. USA 90:10444), and yeast display (Boder, E. T. Wittrup, K. D. (1997) Nat. Biotech. 15:553). Other assays that involve cell-free protein expression have been developed to couple the RNA encoding a protein to the expressed protein including ribosome display (Mattheakis, et al., (1994) Proc. Nat. Acad. Sci. USA 91:9022) and mRNA display (Roberts, R. W. & Szostak, J. W. (1997) Proc. Natl. Acad. Sci. USA 94:12297). Any of these assays or similar assays not listed that couple the DNA or RNA encoding nucleic acid to its expressed protein can be used to isolate cyclic-like peptides, genomic fragments or ScFvs that interact with a protein target.

The invention also providescyclical peptides, ScFv, or genomic fragment isolated as described above.

The present invention provides methods that may be used to generate cyclic and lariat peptide inhibitors of selected targets, which can be used for a variety of purposes. For example, the cyclic peptides can be used as drugs to inhibit disease-causing targets. They can also be used as affinity reagents for validating the therapeutic potential of targets or in general applications that require affinity reagents. In other embodimentembodiments, the lariat peptides are useful for applications that use cyclic peptides, but may also require a tag (tail) to be covalently attached to the cyclic peptide. These tags can encode yeast two hybrid transcription activation domains as described herein. The tags may also encode moieties required for other protein interaction detection systems including: split-ubiquitin system (Stagljar et al., (1998) Proc. Natl. Acad, Sci. USA 95:5187), protein-fragment complementation assay (Remy I. & Michnick S. W. (1999) Proc. Natl. Acad. Sci. USA 96:5394), repressor reconstitution assay (Hirst et al., (2001) Proc. Natl. Acad. Sci. USA 98:8726), SOS recruitment system (Broder et al., (1998) Curr. Boil. 8:1121), phage display (Smith, G. P. (1985) Science 228:1315), bacterial display (Francisco, et al., (1993) Proc. Natl. Acad. Sci. USA 90:10444), and yeast display (Boder, E. T. Wittrup, K. D. (1997) Nat. Biotech. 15:553), ribosome display (Mattheakis, et al., (1994) Proc. Nat. Acad. Sci. USA 91:9022) and mRNA display (Roberts, R. W. & Szostak, J. W. (1997) Proc. Natl. Acad. Sci. USA 94:12297).

The tags may also encode labels for labelling targets (fluorescence, radioactivity etc), localization sequences, membrane permeation sequences, antibody epitope tags, nucleic acid sequences to detect and quantify the amount of bound target, or small molecules. Other suitable uses will of course be apparent to one of skill in the art.

As discussed herein, libraries of lariat peptide can be generated in a variety of organisms. Specific lariat peptides in these libraries that interact with a specific target can be genetically selected using the protein interaction assays described above. The yeast two-hybrid assay has many advantages including but by no means limited to the following.

Cyclic peptides are relatively stable and small, increasing their in vivo stability and cellular permeability. In some embodiments, the invention provides peptides that may be adapted for intracellular peptide delivery. For example, manipulation of the HIV-1-derived Tat-peptide system has been utilized for intracellular peptide delivery. See for e.g. Caron et al. (2001) Intracellular delivery of a Tat-eGFP fusion protein into muscle cells. Mol. Therap. 3(3): 310-18; Wadia and Dowdy (2003) Modulation of cellular function by TAT mediated transduction of full length proteins; and EP 656950 B1. Alternatively, the penetratin, transportan, and MAP (KLAL) peptides can be used to mediate intracellular delivery. See for e.g. Hällbrink et al. (2001) Cargo delivery kinetics of cell-penetrating peptides. Biochim. Biophys. Acta. 1515(2): 101-09; Thorén et al. Uptake of analogs of penetratin, Tat(48-60) and oligoarginine in live cells. Biochem. Biophys. Res. Commun. 307(1): 100-07; WO 2006/101283 A1; and Howl et al. (2003) Intracellular delivery of bioactive peptides to RBL-2H3 cells induces beta-hexosaminidase secretion and phospholipase D activation. Chembiochem. 4(12): 1312-16. Alternatively, oligoarginine fusion proteins can be delivered intracellularly. See for e.g. Han et al. (2001) Efficient intracellular delivery of exogenous protein GFP with genetically fused basic oligopeptides. Mol. Cells. 12(2): 267-71; Futaki et al. (2001) Arginine-rich peptides. An abundant source of membrane-permeable peptides having potential as carriers for intracellular protein delivery. J. Biol. Chem. 276(8): 5836-40; and AU 2003/290511 A8. Alternatively, myristoylated peptides can be delivered intracellularly. See for e.g. Nelson et al. (2007) Myristoyl-based transport of peptides into living cells. Biochemistry 46(51): 14771-81; and EP 651805 B1.

The yeast two-hybrid assay is an easy, fast, and automatable assay. For example, the yeast two-hybrid system can be performed in array format. This allows arrays of lariat peptides to be generated. These arrays can be used to rapidly generate lariat peptides against specific targets using automated robotics. The patterns of lariat peptides that interact with different targets can be used to characterize targets. For example, targets with similar binding surfaces should interact with similar lariat peptides in the array. Alternatively, lariat peptides can be used to pull down target complexes to identify interaction partners. In other embodiments, lariats can be immobilized onto surfaces creating protein micro-array chips to detect protein levels.

In other embodiments, additional functional domains may be attached to the lariat including visualization, and destruction domains.

In specific embodiments, the invention provides recombinant nucleic acid sequences encoding a split intein polypeptide. The split intein polypeptide may include, in amino to carboxy order:

an I_{c} domain comprising an F block and a G block, the F block being at least 80% identical to the sequence rVYDLpV**a--HNFh, designated respectively as positions F1 to F16, and the G block being at least 80% identical to the sequence NGhhhHNp, designated respectively as positions G1 to G8;

an extein domain attached to the C terminal portion of the G block; and,

an I_{N} domain attached to the C terminal portion of the extein domain, the I_{N} domain comprising an A block and a B block, the A block being at least 80% identical to the sequence Ch--Dp-hhh--G, designated respectively as positions A1 to A13, and the B block being at least 80% identical to the sequence G--h-hT-H-hhh, designated respectively as positions B1 to B14. In the foregoing sequences: a capital letter represents an amino acid designated by the single letter amino acid code; “h” represents a hydrophobic residue selected from the group consisting of G, B, L, I, A and M: “a” represents an acidic residue selected from the group consisting of D and E; “r” represents an aromatic residue selected from the group consisting of F, Y and W; “p” represents a polar residue selected from the group consisting of S, T and C; “-” represents any amino acid; and “*” represents optional gaps.

In particular embodiments, which may be characterized by enhanced stability, particularly enhanced stability of a lactone bond in a peptide backbone, various amino acid substitutions may be made in the foregoing formulae, including substitutions in which:
(a) the residue encoded at position G7 is Q, W, F, L, I, Y, M, V, R, K, H, E or D; and/or
(b) the residue encoded at position G6 is L, N, D, W, F, I, M or Y; and/or
(c) the residue encoded at position B11 is K, Y, F, W, H, Q or E; and/or
(d) the residue encoded at position G6 is A and G7 is Y; and/or,
(e) the residue encoded at position G6 is A and B11 is K, Y, F, W, H, Q or E; and/or,
(f) the residue encoded at position F4 is E or Q; and/or,
(g) the residue encoded at position F13 is F, L or I; and/or,
(h) the residue encoded at position F14 is W, F, Y, L, K or R; and/or
(i) the residue encoded at position F15 is W or L; and/or,
(j) the residue at position B9 is not R or T and is a non-catalytic amino acid for an N—X acyl shift; and/or,
(k) the residue at position B10 is not R or T and is a non-catalytic amino acid for an N—X acyl shift; and/or,
(l) the residue at position F2 is not R or T and is a non-catalytic amino acid for an N—X acyl shift; and/or,
(m) the residue at position F6 is not S, T or C and is a non-catalytic amino acid for a transesterification reaction involving a nucleophilic amino acid at position G8 attacking an ester or thioester bond.

In some embodiments, the extein domain may include an immunoglobulin encoding region that encodes an immunoglobulin molecule comprised of a heavy chain variable region attached by linkers to a light chain variable region, a first linker attaching the C-terminal region of the heavy chain variable region to the N-terminal region of the light chain variable region and a second linker attaching the N-terminal region of the heavy chain variable region to the C-terminal region of the light chain variable region, wherein the linkers comprise a polypeptide chain of at least 10 amino acids (or an integer number of amino acids between 10 and 50). In these embodiments, the heavy chain variable region may include one or more heavy chain framework regions selected from the group consisting of HFR1, HFR2, HFR3, and HFR4; and the heavy chain variable region further comprises one or more complementarity determining regions selected from the group consisting of CDR-H1, CDR-H2, CDR-H3; with the heavy chain framework and complementarity determining regions arranged in accordance with the formula HFR1--CDR-H1--HFR2--CDR-H2--HFR3--CDR-H3--HFR4. The light chain variable region may include one or more light chain framework regions selected from the group consisting of LFR1, LFR2, LFR3 and LFR4; and the light chain variable region further comprises one or more complementarity determining regions selected from the group consisting of CDR-L1, CDR-L2 and CDR-L3; with the light chain framework and complementarity determining regions arranged in accordance with the formula LFR1--CDR-L1--LFR27-CDR-L2--LFR3--CDR-L3--LFR4. In these structural formulae:

(i) HFR1 is a first heavy chain framework region consisting of a sequence of about 30 amino acid residues (or any integer value or range therein from 20 to 40);

(ii) HFR2 is a second heavy chain framework region consisting of a sequence of about 14 amino acid residues (or any integer value or range therein from 10 to 30);

(iii) HFR3 is a third heavy chain framework region consisting of a sequence of about 29 to about 32 amino acid residues (or any integer value or range therein from 20 to 50);

(iv) HFR4 is a fourth heavy chain framework region consisting of a sequence of 7 to about 9 amino acid residues (or any integer value or range therein from 5 to 15);

(v) CDR-H1 is a first heavy chain complementary determining region (which may form example be any integer value or range therein from 10 to 100 amino acids is length);

(vi) CDR-H2 is a second heavy chain complementary determining region (which may form example be any integer value or range therein from 10 to 100 amino acids is length);

(vii) CDR-H3 is a third heavy chain complementary determining region (which may form example be any integer value or range therein from 10 to 100 amino acids is length);

(viii) LFR1 is a first light chain framework region consisting of a sequence of about 22 to about 23 amino acid residues (or any integer value or range therein from 15 to 35);

(ix) LFR2 is a second light chain framework region consisting of a sequence of about 13 to about 16 amino acid residues (or any integer value or range therein from 15 to 35);

(x) LFR3 is a third light chain framework region consisting of a sequence of about 32 amino acid residues (or any integer value or range therein from 20 to 40);

(xi) LFR4 is a fourth light chain framework region consisting of a sequence of about 12 to about 13 amino acid residues (or any integer value or range therein from 5 to 25);

(xii) CDR-L1 is a first light chain complementary determining region (which may form example be any integer value from 10 to 100 amino acids is length);

(xiii) CDR-L2 is a second light chain complementary determining region (which may form example be any integer value from 10 to 100 amino acids is length); and,

(xiv) CDR-L3 is a third light chain complementary determining region (which may form example be any integer value from 10 to 100 amino acids is length).

The invention further provides host cells that include the foregoing recombinant nucleic acids, including cells in which the split intein polypeptide is processed in the host cell in a self catalyzed reaction to form at least one cyclized polypeptide having no more than one linear terminal end (such as an immunoglobulin molecule having no more than one linear terminal end and having the conformation of an immunoglobulin fold). For example, the cyclized polypeptide may have one linear terminal end, being a C-terminal end or an N-terminal end, such as a lariat peptide (which may include a lactone or lectern junction). Alternatively, the cyclized polypeptide may be cyclic, so that it has no linear terminal end.

A host cells of the invention may be adapted for use in methods for assaying interactions between fusion proteins. For example, cells of the invention may include:

a first recombinant gene coding for a prey fusion protein, the prey fusion protein comprising a transcriptional repressor or activator domain and a first heterologous amino acid sequence;

a second recombinant gene coding for a bait fusion protein, the bait fusion protein comprising a DNA-binding domain and a second heterologous amino acid sequence; and,

a recombinant reporter gene coding for a detectable gene product, the recombinant reporter gene comprising an operator DNA sequence capable of binding to the DNA binding domain of the bait fusion protein;

wherein expression of the reporter gene is modulated in response to binding between the first heterologous amino acid sequence and the second heterologous amino acid sequence; and,

wherein at least one of the recombinant genes comprises the foregoing recombinante nucleic acids.

In one aspect, the invention provides immunoglobulin molecules having no more than one linear terminal end, including molecules having the conformation of an immunoglobulin fold comprised of a heavy chain variable region attached by linkers to a light chain variable region. In such molecules, a first linker may be present attaching the C-terminal region of the heavy chain variable region to the N-terminal region of the light chain variable region and a second linker may be present attaching the N-terminal region of the heavy chain variable region to the C-terminal region of the light chain variable region. The linkers may be flexible covalent molecular links of at least approximately 50 Angstroms in length, such as polypeptide chains of about 15 amino acids in length, or from 14 to 25 amino acids in length (for example made up of glycine and serine residues).

### BRIEF DESCRIPTION OF THE DRAWINGS

FIG. 1. Intein Catalyzed Protein Splicing Reactions. (a) Self-splicing intein reaction.

Intein domains (black) catalyze a self-splicing reaction that results in the joining of the extein domains (white). (b) Split-intein reaction. The intein is split into two separate proteins. One protein contains the N-Extein and N-Intein and the other protein contains the C-Intein and C-Extein. Interaction between the intein domains results in joining of the extein domains. (c) Split-Intein protein cyclization reaction. The intein domains are swapped relative to the extein domains. Intein domains fold together and catalyze cyclization of the extein domain.

FIG. 2. Schematic of the Intein-Mediated Peptide Cyclization Reaction. Step 1: Intein folding—C-intein and N-intein domains interact to form a catalytically active intein structure. Step 2: N-intein cleavage—Intein catalyzes the cyclization of extein and the cleavage of the N-intein domain. Step 3: C-intein release—C-intein domain is cleaved resulting in the formation of the cyclic peptide.

FIG. 3. Formation of Lariat Intein. (a) The lariat intein is an intermediate product in the intein-catalyzed cyclization reaction. The C-terminal amino acid in the lariat peptide is covalently attached through a lactone bond to a specific nucleophilic residue in the C-Intein domain (I_{c}). (b) The cyclized section of the lariat intein “noose” is used to display (i) random peptides, (ii) genomic fragments, and (iii) antibody single chain variable fragments (ScFv). The I_{c} domain is shown fused to a nuclear localization sequence (NLS), transcription activation domain (ACT), haemagglutinin tag (HA).

FIG. 4. Formation of Unprocessed Intein. (a) The unprocessed intein is formed by blocking Step (i) in the intein-catalyzed cyclization reaction. (b) The extein or region constrained between the C-Intein (I_{c}) and N-Intein (I_{N}) domains is used to display (i) random peptides, (ii) genomic fragments, and (iii) antibody single chain variable fragments (ScFv). The I_{c} domain is shown fused to a nuclear localization sequence (NLS), transcription activation domain (ACT), and haemagglutinin tag (HA).

FIG. 5. Formation of the Dicysteine intein. (a) The dicysteine intein is formed by blocking Step (i) in the intein-catalyzed cyclization reaction. The dicysteine intein contains one Cys after the C-Intein domain (l_{c}) and one Cys at in the first amino acid position of the N-Intein domain (I_{N}). (b) The extein, or region constrained between the two Cys amino acids is used to display (i) random peptides, (ii) genomic fragments, and (iii) antibody single chain variable fragments (ScFv). The I_{c} domain is shown fused to a nuclear localization sequence (NLS), transcription activation domain (ACT), and haemagglutinin tag (HA).

FIG. 6. Conversion of Lariat, Unprocessed, and Dicysteine intein to Cyclic and Linear Peptides. (a) The lactone-cyclized peptide or protein in the lariat intein can be converted to a head to tail cyclized peptide or protein or a linear peptide or protein. (b) The constrained peptide or protein in the unprocessed intein can be converted to a head to tail cyclized peptide or protein or a linear peptide or protein. (c) The constrained peptide or protein in the dicysteine intein can be converted to a Cys cross-linked or disulfide bond cyclized peptide or protein or a linear peptide or protein. I_{N} is the N-Intein domain and I_{C} is the C-Intein domain. The I_{C} domain is shown fused to a nuclear localization sequence (NLS), transcription activation domain (ACT), and haemagglutinin tag (HA).

FIG. 7. Yeast Two-Hybrid Assay Using the Lariat, Unprocessed, and Dicysteine intein. (a) Combinatorial lariat intein libraries are screened using the yeast two-hybrid assay. The lariat intein contains a transcription activation domain (ACT) fused to the I_{C} domain, which is required to activate the reporter genes in the yeast two-hybrid assay. (b) Combinatorial unprocessed intein libraries are screened using the yeast two-hybrid assay. The unprocessed intein contains a transcription activation domain (ACT) fused to the I_{C} domain, which is required to activate the reporter genes in the yeast two-hybrid assay. (c) Combinatorial dicysteine intein libraries are screened using the yeast two-hybrid assay. The dicysteine intein contains a transcription activation domain (ACT) fused to the I_{C}domain, which is required to activate the reporter genes in the yeast two-hybrid assay. For all examples listed above, the I_{C}domain is also shown fused to a nuclear localization sequence (NLS), and haemagglutinin tag (HA).

FIG. 8. Numbering of Intein and Extein Amino Acids. The C-Intein domain (I_{C}) has conserved blocks F and G, and the N-Intein (I_{N}) domain has conserved blocks A, and B. Conserved amino acids are numbered according to their block letter and position number. An enlargement of the splice site at the I_{C}-Extein-I_{N} boundaries is shown. The I_{C}intein is numbered from C-terminus to N-terminus using the labelling scheme I_{C-1}, I_{C-2}, I_{C-3} . . . , or according to block letter and position number. Block G is number 1-8 and Block F is numbered 1-16. The I_{N} intein is numbered from the N-terminus to the C-terminus using the labelling scheme I_{N+1}, I_{N+2}, I_{N+3} . . . , or according to block letter and position number. Block A is numbered 1-13 and Block B is numbered 1-14. The extein is numbered from N-terminus to C-terminus using the labelling scheme I_{C+1}, I_{C+2}, I_{C+3} . . . , or from C-terminus to N-terminus using the labelling scheme I_{N-1}, I_{N-2}, I_{N-3} . . . . The consensus sequence for each block is shown below the block. Definitions: h=hydrophobic residues (G, V, L, I, A, M); a=acidic residues (D,E); r=aromatic residues (F,Y,W); p=polar residues (S, T, C); “•”=non-conserved residue; “*”=gap introduced for better alignment; Capital Letter=single letter amino acid code, representing a highly conserved position.

FIG. 9. Standard Mechanism for intein-Mediated Protein Splicing. Step (i): N—X acyl shift—The I_{N+1} (A1) nucleophile at the Extein-I_{N} junction undergoes and N—X acyl shift to convert the amide bond to an ester or thioester. Step (ii): Transesterification reaction—The I_{C+1} (G8) nucleophile at the I_{C}-Extein junction undergoes a nucleophilic attack on the ester or thioester formed in Step (i) and produces the branched intermediate. Step (iii): Asn cyclization—The I_{C+2} Asn undergoes side chain cyclization, which cleaves amide bond between the I_{C}domain and the extein, generating exteins joined by an ester or thioester. Step (iv): Ester to amide shift—The ester or thioester bond is converted to an amide bond by the thermodynamically favoured X to N acyl shift. Definitions: X═O or S depending on Ser or Cys. I_{N} is the N-Intein domain and I_{C}is the C-Intein domain.

FIG. 10. Non-Standard Mechanism for Intein-Mediated Protein Splicing. Step (i): Direct attack on amide bond—The I_{C+1} (G8) nucleophile at the I_{C}-Extein junction undergoes a nucleophilic attack on the amide bond connecting the N-Extein and I_{N} domain and produces the branched intermediate. Step (ii): Asn cyclization—The I_{C+2} Asn undergoes side chain cyclization, which cleaves the I_{C} domain and generates the extein product joined by an ester or thioester. Step (iii): Ester to amide shift—The ester or thioester is converted to the amide by the thermodynamically favoured X to N acyl shift. Definitions: X═S or O depending on Cys, or Ser/Thr. I_{N} is the N-Intein domain and I_{C}is the C-Intein domain.

FIG. 11. Intein-Mediated Protein Cyclization Reaction. Step (i): N—X acyl shift—The I_{N+1} (A1) nucleophile at the Extein-I_{N} junction undergoes and N—X acyl shift to convert the amide bond to an ester or thioester. Step (ii): Transesterification reaction—The I_{C+1} (G8) nucleophile at the I_{C}-Extein junction undergoes a nucleophilic attack on the ester or thioester formed in Step (i) and produces the branched intermediate. Step (iii): Asn cyclization—The I_{C+2} Asn undergoes side chain cyclization, which cleaves the I_{C} domain and generates the extein product as a lactone. Step (iv): Lactone to Lactam Shift—The lactone cyclized intein is converted to the lactam by the thermodynamically favoured X to N acyl shift. Definitions: X═S or O depending on Cys, or Ser/Thr. I_{N} is the N-Intein domain and I_{C}is the C-Intein domain.

FIG. 12. Generation of the Unprocessed Intein. (a) The unprocessed intein is generated by inhibiting Step (ii) (Transesterification reaction). If only Step (ii) is blocked then the unprocessed intein can undergo two side reactions. Side reaction (iii) (Asn cyclization) causes the I_{C} domain to be cleaved from the unprocessed intein. Side reaction (iv) (Ester hydrolysis) cause cleavage of the I_{N} domain from the unprocessed intein. To generate a stable unprocessed intein Steps (i) (N—X acyl shift) and (iii) (Asn cyclization) need to be inhibited. The I_{C}domain is shown fused to a nuclear localization sequence (NLS), transcription activation domain (ACT), and haemagglutinin tag (HA). X═S or O depending on Cys, or Ser/Thr.

FIG. 13. Generation of the Dicysteine Intein. (a) The dicysteine intein is generated by inhibiting Step (ii) (Transesterification reaction). If only Step (ii) is blocked then the unprocessed intein can undergo two side reactions. Side reaction (iii) (Asn cyclization) causes the I_{C}domain to be cleaved from the unprocessed intein. Side reaction (iv) (Ester hydrolysis) cause cleavage of the I_{N} domain from the unprocessed intein. To generate a stable dicysteine intein Steps (I) (N—X acyl shift) and (iii) (Asn cyclization) need to be inhibited. The I_{C}domain is shown fused to a nuclear localization sequence (NLS), transcription activation domain (ACT), and haemagglutinin tag (HA). X═S or O depending on Cys, or Ser/Thr.

FIG. 14. Generation of the Lariat Intein. (a) To generate the lariat intein Step (iii) (Asn cyclization) needs to be blocked. The lariat intein can undergo the side reaction (iv) (Lactone hydrolysis). To generate a stable intein lariat, Step (iv) (Lactone hydrolysis) should be reduced. The I_{C}domain is shown fused to a nuclear localization sequence (NLS), transcription activation domain (ACT), and haemagglutinin tag (HA). X═S or O depending on Cys, or Ser Thr.

FIG. 15. Isolation of Anti-LexA lariats. (a) Intein-mediated peptide cyclization. (i) Unprocessed intein undergoes an N-to-S acyl shift using the I_{N+1} cysteine at the peptide-I_{N} junction. (ii) Transesterification reaction involving I_{C+1} serine at the l_{C}-peptide junction and the thioester formed in step (i), which releases the I_{N} domain producing the lariat intermediate. (iii) In the intein producing cyclic peptide system, I_{C+2} asparagine undergoes a side chain cyclization, which releases the I_{C}domain and generates a lactone-cyclized peptide that undergoes a thermodynamically favoured O to N acyl shift to produce a lactam-cyclized peptide. In the lariat producing intein, asparagine at position I_{C}−1 is mutated to alanine (*), which inhibits asparagines cyclization. (b) Mutations used to produce lariat and inactive inteins. Lariat intein contains an asparagine to alanine mutation at position I_{C-1}, which blocks the asparagine side chain cyclization reaction. Inactive intein contains the same mutations as the lariat intein and a serine to alanine mutations at position l_{C+1} and a cysteine to alanine mutation at position I_{N+1}. Cysteine to alanine mutation at I_{N+1} blocks the N to S acyl shift. Serine to alanine mutation at I_{C+1} blocks the transesterification reaction. X represents amino acids coded by the NNK codon. (c) Lariat yeast two-hybrid assay. The asparagine side chain cyclization reaction is inhibited by mutating asparagine to alanine, which stops the cyclization reaction at the lariat intermediate. The lariat contains a transcription activation domain, which is used to select anti-LexA lariats using the yeast two-hybrid interaction trap. (d) Amino acid sequences of the noose region from two anti-LexA lariat peptides (L1 and 12). Amino acids from the combinatorial region are bolded and dashes are used to align common amino acids in L1 and L2.

FIG. 16. Analysis of Combinatorial Lariat Library. Sequences from seventeen lariat library plasmids (pIL-XX). Bold amino acids are constant. * represent stop codons. X represents amino acids coded by the NNK codon. 35% of the library contains random seven amino acids peptides with no stop codons.

FIG. 17. Analysis of L2 Lariat. (a) Western analysis of intein-mediated lariat production in EY93 using an anti-HA antibody. pIL-L2 and pIN-L2 are designed to produce lariat and unprocessed inteins, respectively. The unprocessed intein is at ˜23 kDa and the lariat is at ˜9 kDa. (b) Yeast two-hybrid analysis of the L2 lariat interaction with LexA. pIL-01 is a lariat expression plasmid with a CGPC peptide noose. pIL-L2 is a lariat expression plasmid with an L2 noose. pIN-L2 is a mutant lariat expression plasmid with L2 noose that produces only the unprocessed intein. (i) Yeast growth on nonselective His⁻,Trp⁻ glucose media. (ii) Yeast grown on His⁻Tip⁻Leu⁻Ade⁻Xgal galactose/sucrose media that selects for the activation of LEU2, ADE2, and LacZ yeast two-hybrid reporter genes. (c) HPLC and ESI-TOF MS analysis of His-tag purified lariat produced in BL21-CP. (i) Reverse-phase HPLC separation of the lariat and I_{N} fragment. (ii) ESI-TOF MS analysis of lariat (8651.7 calc; 8651.4 obs) and hydrolyzed lariat (8669.7 calc; 8669.5 obs). (iii) ESI-TOF MS analysis of I_{N} fragment (13966.7 calc: 13967.0 obs). (d) Analysis of the amount of lariat present prior to MS analysis. (i) Lariat cyclized through a lactone bond. (ii) Lariat cleaved prior to Na¹⁸OH treatment. (iii) Products from the Na¹⁸OH induced cleavage of lactone bond. (iv) Trypsin digest of cleaved lariat to confirm the location of ¹⁸O incorporation. The percentage of each fragment containing ¹⁸O is shown and corresponds to the amount of lariat cyclized through a lactone bond prior to MS analysis (FIG. 20).

FIG. 18. Surface Plasmon Resonance (SPR) Analysis of L2 interaction With LexA. L2 peptide was immobilized onto a CM5 sensor chip and LexA (11 μM-110 μM) was passed over the sensor chip. The response curve of each point was used to determine the dissociation constant (K_{d}) using the BiaEvaluation (Biacore) fitting software. Standard deviation was calculated using the different LexA concentrations.

FIG. 19. Mechanism for Lariat Cleavage By NaOH. Hydrolysis of the lariat lactone by Na¹⁸OH can occur by two mechanisms. (a) The first mechanism involves the hydrolysis of the ester bond causing ¹⁸O incorporation at the tyrosine carboxylic acid at position (I_{N-1}). (b) The second mechanism involves an α-H elimination to generate dihydroalanine, followed by a Michael addition, which incorporates the O¹⁸ at the serine side chain at position (I_{C+1}).

FIG. 20. Quantification of Lariat Prior to MS analysis. (a) Analysis of ¹⁸O incorporation at the tyrosine carboxylic acid at position (I_{N-1}). Trypsin digest of the Na¹⁸OH treated lariat produces a peptide fragment containing tyrosine at position (I_{N-1}) (SWDLPGEY). The ¹⁶O product has a calculated mass of 966.420 m/z and the ¹⁸O product has a calculated mass of 968.420 m/z. (b) Mass spectrometry analysis of SWDLPGEY peptide fragment from the Na¹⁶OH or Na¹⁸OH treated samples over layed with the theoretical isotope distribution (MS-ISOTOPE software). In the Na¹⁸OH treated sample there is a large deviation from the theoretical distribution indicating the presence of more than one peptide. (c) MATCHING software analysis of the percentages of ¹⁶O labelled peptide (966.3 m/z, 86%, squares) and ¹⁸0 labelled peptide (969.3 m/z, 14%, triangles) in the observed spectrum. The 1+ and 2+ charged fragments were analyzed and similar results were observed. Only the 1+ charge is shown. (d) Overlay of the sum of the calculated contributions of the ¹⁸O and ¹⁸O peptides on the observed SWDLPGEY peptide fragment spectrum. (e) Analysis of ¹⁸O incorporation at the serine side chain at position (I_{C+1}). Trypsin digest of the Na¹⁸OH treated lariat produces a peptide fragment containing serine at position (I_{C+1}) (IFDIGLPQDHNFLLANGAIAHASR). The mass of this fragment is 2590.352 m/z corresponding to a product 1 Da heavier than the predicted ¹⁸O incorporated product. A 1 Da shift can be attributed to deamidation of asparagine. The asparagine at position (I_{C}.₇) is susceptible to base-catalyzed deamidation as it is N-terminal to a glycine (7-10). The 2+, 3+4+and 5+charged fragments were analyzed and similar results were obtained. Only the 4+ charged fragment is shown. (f) Mass spectrometry analysis of IFDIGLPQDHNFLLANGAIAHASR peptide fragment from the Na¹⁶OH or NA¹⁸OH treated samples overlayed with the theoretical isotope distribution (MS-ISOTOPE software). In the Na¹⁸OH treated sample there is a large deviation from the theoretical distribution indicating the presence of more than one peptide. The Na¹⁸OH treated sample should incorporate two ¹⁸O, one from the hydrolysis and the second from deamidation, resulting in a M+H of 2595.36 Da. (g) MATCHING software analysis of the percentages of ¹⁶O and ¹⁸O labelled peptides: (▪) a peptide with two ¹⁶O substitutions corresponding to deamidation and hydrolysis by ¹⁸O (2591.35 m/z, 8.8%, squares), (▴) a peptide with one ¹⁶O and one ¹⁸O substitution corresponding to deamidation by ¹⁸O and hydrolysis by ¹⁶O (2593.35 m/z, 59.0%, triangles), and () a peptide with two ¹⁸O substitutions (2595.35 m/z, 32.2%, circles) corresponding to deamidation and hydrolysis by ¹⁸O. D 32 deamidation and H=hydrolysis. (h) Overlay of the sum of the calculated contributions of the ¹⁸O and ¹⁶O peptides on the observed IFDIGLPQDHNFLLANGAIAHASR peptide fragment spectrum.

FIG. 21. Biological Activity of L2 Lariat and Cyclic Peptide. (a) Inhibition of MMC-induced LexA cleavage by L2 lariat. 13L21-CP cells expressing either pETIL-01, which expresses a lariat with a CGPC noose, or pETIL-L2, which expresses a lariat with an L2 noose, were treated with MMC and chloramphenicol. Cell extracts were analyzed by Western analysis using Anti-LexA antibody at 0, 1, 2, and 3 hours after MMC addition. (b) Inhibition of MMC-induced expression of sulA-GFP in SMR6039-DE3. Percentage of GFP expressing SMR6039-DE3 cells transformed with pETIL-L2 and treated with MMC in the presence and absence of IPTG. GFP expression was analyzed at 0, 0.5, 1.5, and 2.5 hours after MMC addition using flow cytometry. Error bars represent the standard deviation from three independent experiments. (c) Survival assay for BL21-CP cells transformed with pETIL-01 or pETIL-L2 in the presence (+) and absence (−) of MMC and/or IPTG. (d) Survival assay for BL21-CP cells treated synthetic linear and cyclic L2 peptides. Normalized percent cell survival is calculated by dividing the number of colony forming units (cfu) after one hour by the number of cfu at the zero hour time point. The uninduced control or the no peptide control is normalized to 100%. Error bars represent the standard deviation of three independent experiments.

FIG. 22. Linear U Peptide Inhibits Cell Survival and Potentiates Mitomycin C Activity. Survival assay for BL21-CP cells treated synthetic linear L2 peptide. Cell survival is reported relative to the untreated control. Error bars represent the standard deviation of three independent experiments.

FIG. 23. Oligonucleotides Used To Construct Lariat Intein and Mixed Intein.

FIG. 24. The affect of lariat mutations on lariat stability and processing. Three positions in the intein construct, G6, G7, and B11 were mutated to amino acids listed. The wild-type intein process all steps in the intein reaction and produces a cyclic peptide. The G6: His, G7: Ala, and B11: Arg is the lariat producing intein construct. (−) indicates the lariat formation and processing was not characterized. % lariat is the amount to unhydrolyzed lariat. % processing is the amount of undergoing the first two steps in the lariat reaction.

FIG. 25. Amino Acid Positions in The Diversified Complementarity Regions (CDRs) Of The ScFv Libraries. The names of the CDRs are listed above the tables and the positions are labelled with numbers corresponding to the Kabat database. The letters under the numbers refer to the amino acids in that position in the single letter amino acid code. X denotes a variable position.

FIG. 26. Time course analysis of ScFv lariat processing. Western analysis of intein-mediated ScFv lariat production in EY93 using an anti-HA antibody. The unprocessed intein is at ˜54 kDa and the lariat is at ˜42 kDa.

FIG. 27. Yeast two-hybrid comparison of ScFv library interactions. K4, cyc-K4, T4 and cyc-T4 libraries were screened against a pool of five baits: Bcr-Abl SH2 Domain, Bcr-Abl SH3 Domain, Bcr-Abl Coiled-coil domain, Bcr-Abl Y177 Motif, and Hck Tyr Kinase Domain. The number of positive colonies growing due to activation of the Adenine reporter (ADE) is shown in black bars. The number of positive colonies growing and turning blue due to activation of the Adenine reporter (ADE) and the LacZ reporter are shown in grey bars. Errors bars represent the standard deviation from five independent experiments.

FIG. 28. Oligonucleotides used to construct ScFv Libraries

### DETAILED DESCRIPTION

Head to tail peptide cyclization, resulting in a continuous amide peptide backbone, has been successfully used to constrain and stabilize peptides and to improve their biological activity. A variety of in vitro chemical and enzymatic strategies for cyclizing peptides from their linear precursors have been developed. Recently, methods using inteins have been developed to synthesize head to tail cyclic peptides in vivo (Scott, C. P. et al. (1999) Proc. Natl. Acad. Sci. USA 96:13638-13643).

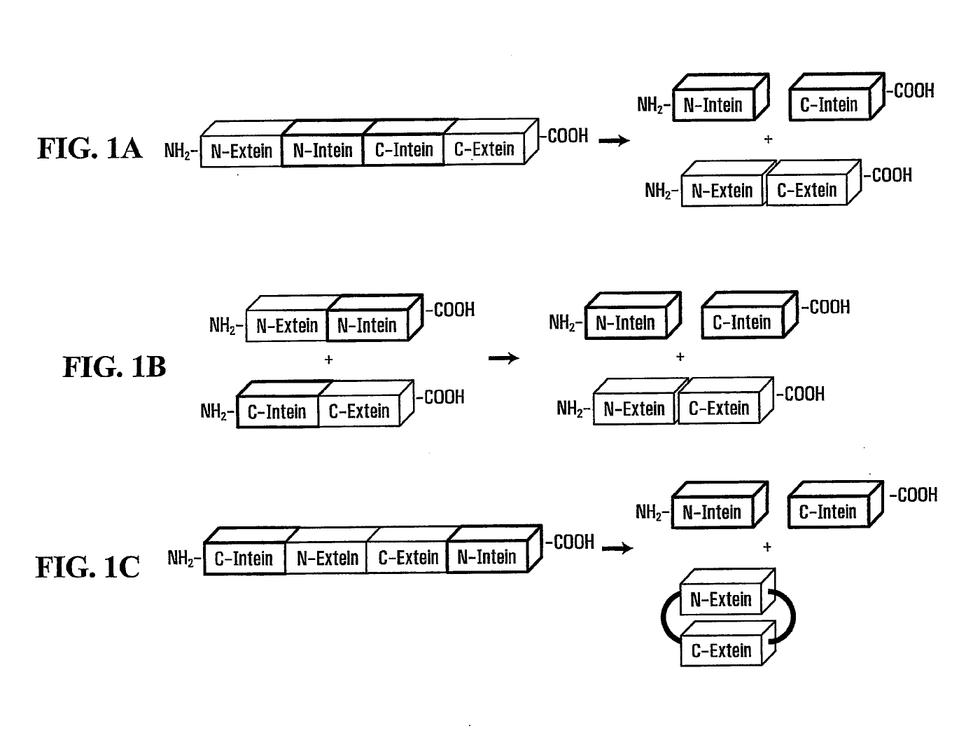

Inteins are self-splicing proteins that are present in between exteins in a precursor protein. Inteins remove themselves from the precursor protein, resulting in a joining of the exteins (FIG. 1a). Naturally occurring and engineered inteins and split inteins ligate proteins and peptides together (FIG. 1b). Based on these results, inteins have been further engineered to generate cyclic proteins and peptides. To do this, the order of the intein domains are changed (FIG. 1c) to enable the head to tail cyclization of the extein domain.

Methods for using engineered inteins to create head to tail cyclized peptides have been described. Combinatorial cyclic peptide libraries have been generated by fusing random peptides between intein domains. These cyclic peptide libraries have been used to isolate specific head to tail cyclized peptides that block Interleukin-4 signalling in human B-cells (Kinsella, T. M. et al. (2002) J. Biol. Chem. 277:37512-37518). Although this method has been used successfully to alter cell phenotypes, it is difficult to determine the cellular targets of the cyclic peptides. Recently, techniques have been developed to isolate head to tail cyclized peptides that disrupt specific protein-protein interaction using a genetic selection strategy in bacteria (Horswill, A. R. et al. (2004) Proc. Natl. Acad. Sci. USA 101:15591-15596, Tavassoli, A. & Benkovic, S. J. (2005) Agnew Chem. int. Ed. Engl. 44:2760-2763). However, as will be apparent to one of skill in the art, head to tail cyclized peptides lack free N-terminal or C-terminal ends which means that reporters or activators cannot be attached thereto.

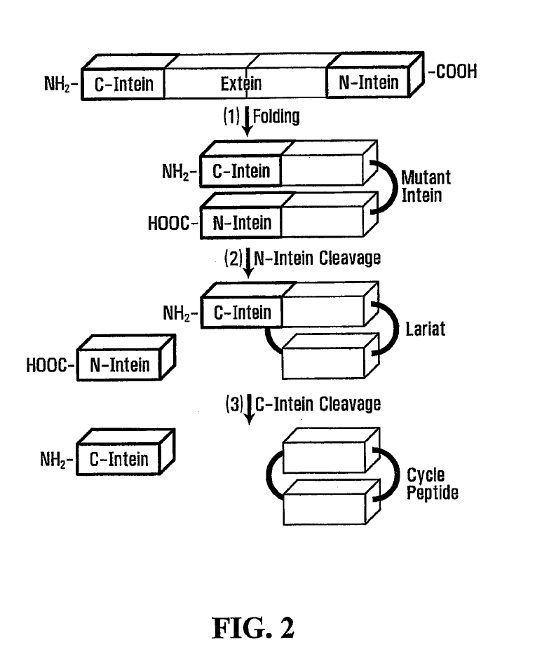

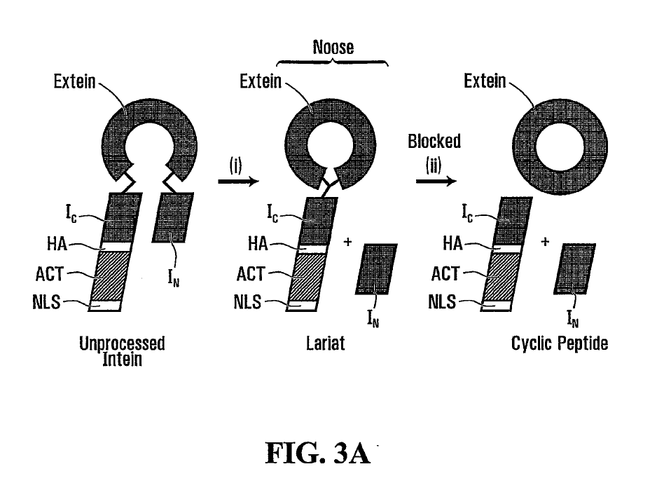

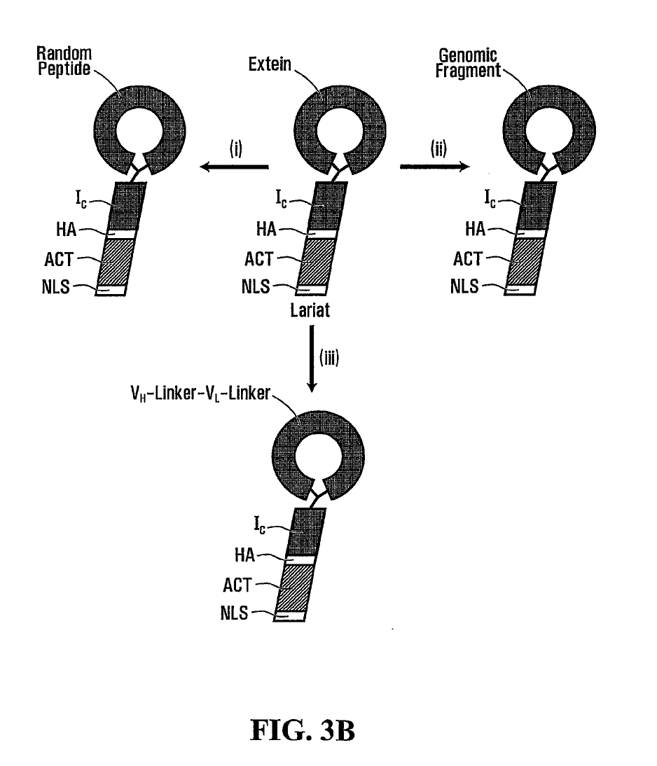

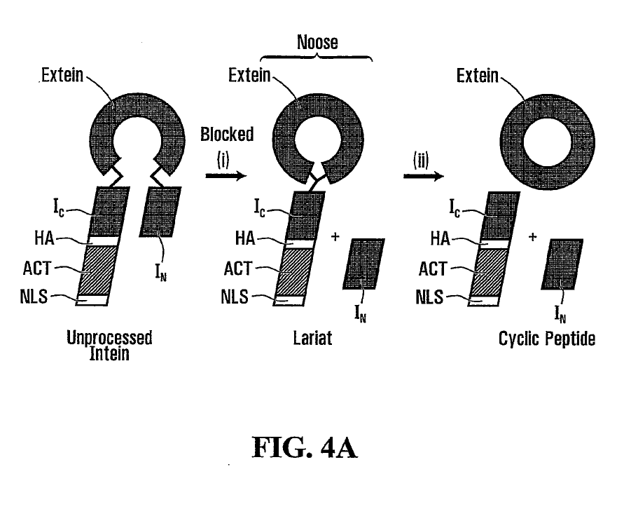

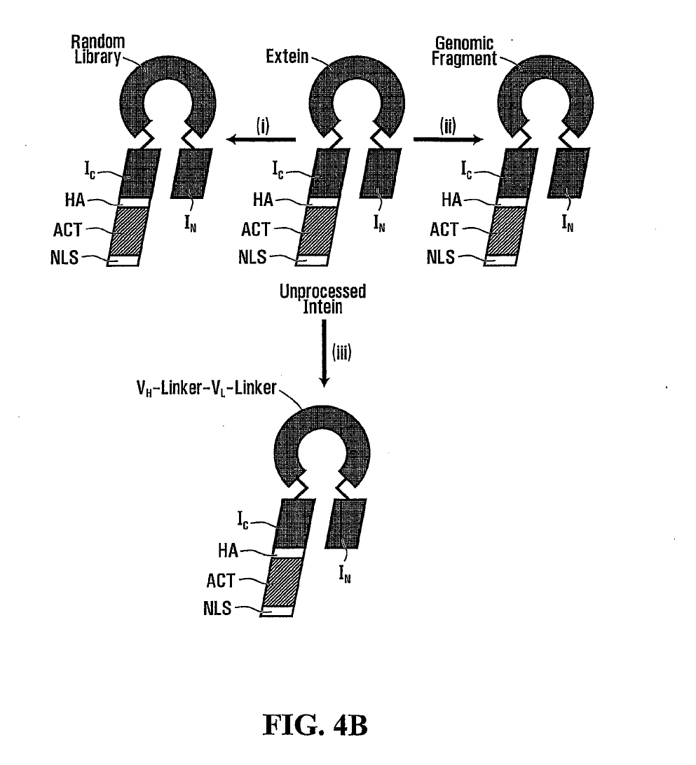

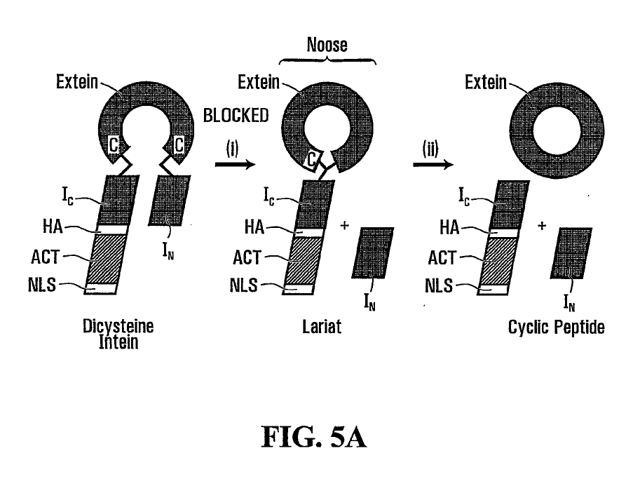

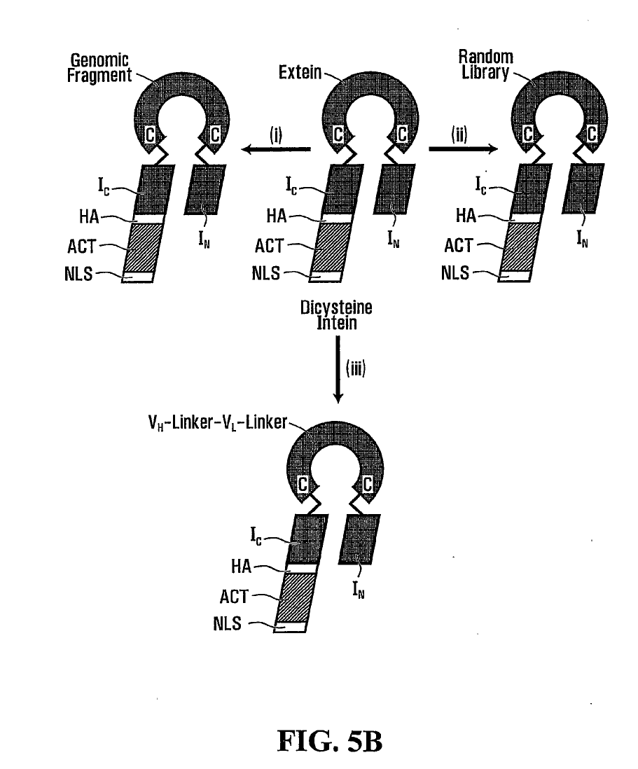

The present invention describes the construction and application of the “lariat” intein or lariat precursors (unprocessed or dicysteine inteins) in the yeast two-hybrid assay or other selection technologies described above and/or known in the art. The lariat is a new peptide construct that has no C-terminus and represents a novel class of cyclic peptides. Lariat inteins are generated by modifying the in vivo intein-mediated protein ligation reaction (FIG. 2). The C-terminus of the lariat intein is looped back and linked to a specific serine in the interior of the peptide via a lactone bond (FIG. 3). Libraries of random peptides, ScFvs, or genomic fragments can be displayed in the cyclic or noose region of the lariat (FIG. 3). The lariat, unlike a head to tail cyclized peptide, has a free N-terminus that allows the attachment of useful activity domains such as a transcription activation domain, which is necessary for yeast-two hybrid assays. The unprocessed intein (FIG. 4) is an intein construct that is unable to undergo any steps in the intein mediated cyclization. Random peptides, ScFvs, or genomic fragments can be displayed and constrained between the C- and N-intein domains (FIG. 4). The unprocessed intein has a free N- and C-terminus that allows the attachment of useful activity domains at either end such as a transcription activation domain, which is necessary for yeast-two hybrid assays. The dicysteine intein (FIG. 5) is an intein construct that is unable to undergo any steps in the intein mediated cyclization. Random peptides, ScFvs, or genomic fragments can be displayed and constrained between the C- and N-intein domains (FIG. 5). In the dicysteine intein, random peptides, ScFvs, or genomic fragments are flanked by a Cys at each end. The dicysteine intein has a free N- and C-terminus that allows the attachment of useful activity domains at either end such as a transcription activation domain, which is necessary for yeast-two hybrid assays. The lariat construct or the unprocessed intein and dicysteine intein can be used to display libraries of combinatorial cyclic peptides, ScFvs, or genomic fragments fused to a transcription activation domain. Here, ScFv refers to an antibody fragment consisting of immunoglobulin variable (V) domains of heavy (H) and light (L) chains held together by a short linker (Tanaka, T. et al. (2003) Nucl. Acids Res. 31:e23). Note that in FIGS. 3, 4, and 5 that the ScFv can be constructed with the V_{H} domain fused to the I_{C}domain followed by a linker and the V_{L} domain. Alternatively, the V_{L} domain can be fused to the I_{C}domain followed by a linker and the V_{H} domain. As used herein “ScFv” refers to either construct. Here genomic fragments refer to randomly or rationally generated fragments of DNA derived from genomic DNA or cDNA that are expressed in the lariat, unprocessed intein or dicysteine intein constructs. The yeast two-hybrid assay or other selection technologies can be used to genetically select lariat peptides, unprocessed inteins, and dicysteine inteins that bind to specific targets.

Other assays for detecting protein, RNA, and DNA interactions within cells can also be used to select lariat peptides, unprocessed inteins, and dicysteine inteins that bind to specific targets including two-hybrid systems (reviewed in Vidal M. & Legrain P. (1999) Nucl. Acid Res. 27:919), split-ubiquitin system (Stagljar et al., (1998) Proc. Natl. Acad. Sci. USA 95:5187), protein-fragment complementation assay (Remy I. & Michnick S. W. (1999) Proc. Natl. Acad. Sci. USA 96:5394), repressor reconstitution assay (Hirst et al., (2001) Proc. Natl. Acad. Sci. USA 98:8726), and SOS recruitment system (Broder et al., (1998) Curr. Biol. 8:1121). Any of these assays or similar assays not listed that detect protein, RNA, or DNA interactions using reporter genes/proteins in cells can be used to isolate cyclic-like peptides, genomic fragments or ScFvs that interact with a target. Alternatively, many assays have been reported that couple the DNA encoding a protein to the expressed protein including phage display (Smith, G. P. (1985) Science 228:1315), bacterial display (Francisco, et al., (1993) Proc. Natl. Acad. Sci. USA 90:10444), and yeast display (Boder, E. T. Wittrup, K. D. (1997) Nat. Biotech. 15:553). Other assays that involve cell-free protein expression have been developed to couple the RNA encoding a protein to the expressed protein including ribosome display (Mattheakis, et al., (1994) Proc. Nat. Acad. Sci. USA 91:9022) and mRNA display (Roberts, R. W. & Szostak, J. W. (1997) Proc. Natl. Acad. Sci. USA 94:12297). Any of these assays or similar assays not listed that couple the DNA or RNA encoding nucleic acid to its expressed protein can be used to isolate cyclic-like peptides, genomic fragments or ScFvs that interact with a target.

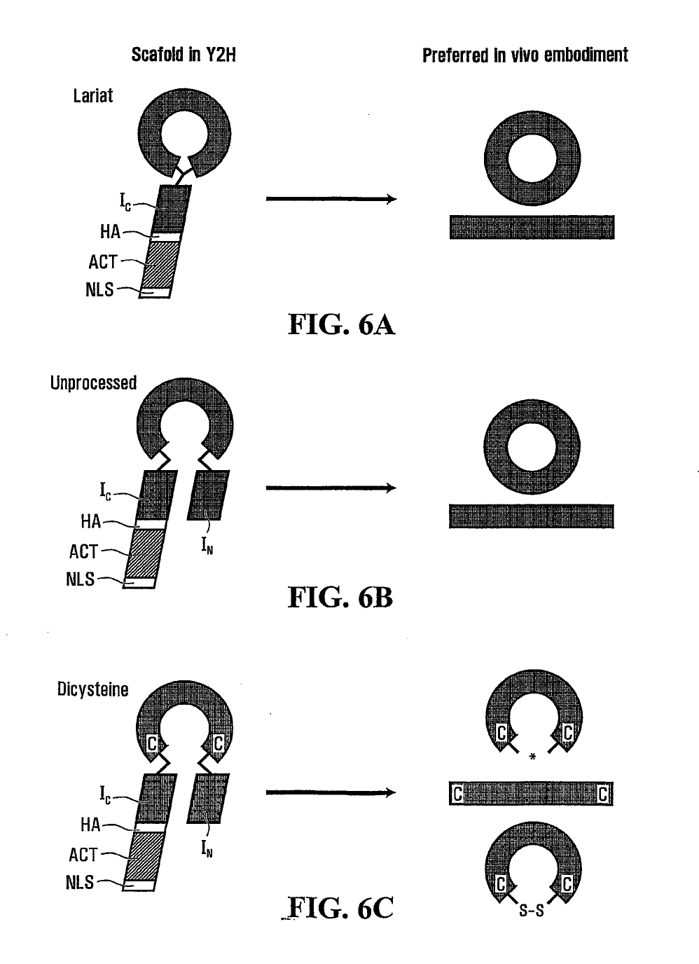

The lariat and unprocessed inteins that bind specific targets can be used as templates for synthesizing linear or cyclic peptides, ScFvs or genomic fragments that interact with the same target but do not contain any intein sequence (FIG. 6a, b). Dicysteine inteins that interact with a target can be used to design constrained peptides, ScFvs or genomic fragments that interact with the target, but that do not contain any of the intein sequence. To do this, the peptide, ScFv, or genomic fragment that are displayed between the C- and N-intein domains are synthesized with flanking Cys. The flanking Cys are used to cross-link and constrain the peptide, ScFv, or genomic fragment (FIG. 6c). The dicysteine intein can also be constructed using other cross-linkable amino acids in place of the two-cysteine residues. Examples of cross-linkable moieties present on amino acids include but are not limited to: amine-thiol, amine-amine, amine-carboxylic acid, carboxylic acid-carboxylic acid, etc. Further amino acids can be post-translationally modified to incorporate cross-linkable moieties that are not naturally present on amino acids. Further the cross-linking molecules can be designed such that additional molecules with unique functions can be appended to the peptide, ScFv, or genomic fragment. These molecules may include fluorescent labels, localization sequences, purification tags, molecule destruction moieties, etc.

The term ‘intein’ refers to a well-known group of ‘splicing proteins’. As discussed herein, a variety of inteins can be modified as discussed below for use in the invention. The N-intein domain and C-intein domain from different inteins can also be mixed to create functional inteins. Examples include but are not limited to naturally occurring split-inteins, for example, Aha DnaE-c and Aha DnaE-n, Aov DnaE-c and Aov DnaE-n, Asp DnaE-c and Asp DnaE-n, Ava DnaE-c and Ava DnaE-n, Cwa DnaE-c and Cwa DnaE-n, Dra Snf2-c and Dra Snf2-n, Npu DnaE-c and Npu DnaE-n, Nsp DnaE-c and Nsp DnaE-n, Oli DnaE-c and Oli DnaE-n, Ssp DnaE-c and Ssp DnaE-n, Tel DnaE-c and Tel DnaE-n, Ter DnaE-3c and Ter DnaE-3n and Tvu DnaE-c and Tvu DnaE-n.

Other suitable inteins include those peptides identified as being an intein, that is, a peptide that meets the following criteria (from a New England Biolabs Webpage):

1) An in-frame insertion in a gene that has a previously sequenced homolog lacking the insertion.

2) The observed size of the mature protein is similar to the size of homologs lacking the intein and not to the predicted size of the precursor. Many groups have gone a step further to prove protein splicing by amino acid sequencing across the splice junction in the ligated exteins or by identifying spliced peptides by mass spec analysis. In the absence of experimental proof of splicing, inteins should be considered putative and are marked theoretical in the Intein Registry.

3) The presence of intein splicing motifs consisting of Blocks A, N2, B, N4, F and G. Although Blocks C, D, E and H are part of the endonuclease domain, they tend to be more conserved than the splicing motifs and are sometimes easier to find in a candidate sequence. However, the presence of homing endonuclease domains is insufficient to classify a protein as an intein, since many homing endonucleases are free-standing or found in introns. Mini-inteins that lack these DOD motifs are thus harder to identify, especially when they contain non-consensus sequences in conserved positions. Note that recent papers have reported ‘protein splicing’ that is not intein-mediated, nor is it self-catalytic. Please distinguish between intein-mediated protein splicing and other Protein Editing mechanisms that result in spliced, rearranged proteins.

4) The presence of the four conserved splice junction residues: Ser, Thr or Cys at the intein N-terminus The dipeptide His-Asn or His-Gln at the intein C-terminus Ser, Thr or Cys following the downstream splice site. Ser, Thr, Cys and Asn are essential residues that act as nucleophiles in the splicing pathway. The absence of these residues or the substitution with residues that cannot perform similar chemistry, would suggest an inactive intein or an alternate splicing pathway. Thr has not been observed at the intein N-terminus, but can effectively substitute for Ser in the Tli Pol-2 intein (Hodges, R. A. et al. (1992) Nucl. Acid. Res. 20:6153-6157). The conserved Thr (Block B) and His (in Blocks B and G) residues assist in catalysis and thus may not be essential since other residues in the intein may provide similar facilitating functions in their absence.

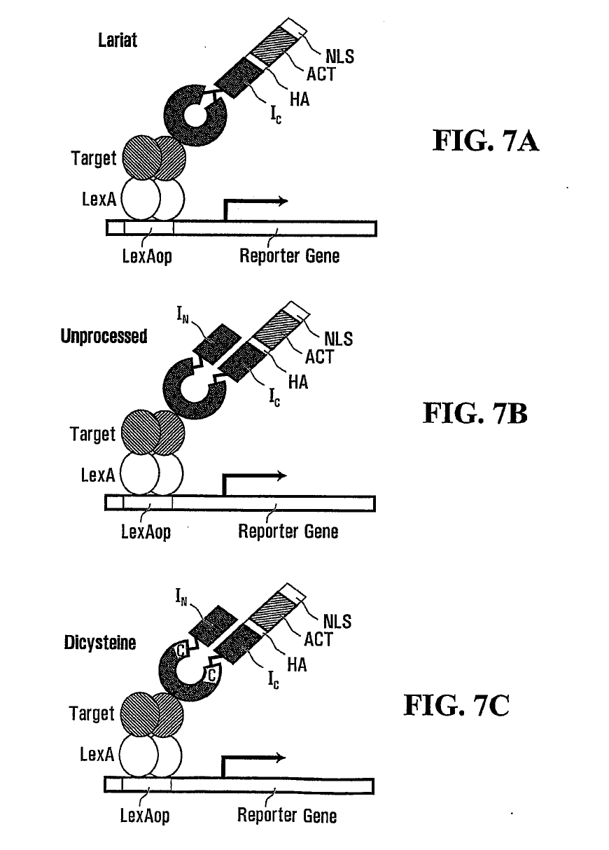

In one aspect, the invention provides methods for isolating lariat inteins, unprocessed inteins, and dicysteine inteins that recognize a selected target, for example using the yeast two-hybrid interaction trap (FIG. 7). Lariat inteins are cyclized peptides, genomic fragments, or ScFvs that have a peptide tag covalently attached to the cyclized or noose region. Lariat peptides are generated by mutating the cyclic-peptide generating intein such that it only undergoes the first two steps in the cyclization reaction. A lariat is an intermediate product in the intein-mediated cyclic peptide reaction. The lariat product contains a tail (for the yeast two-hybrid assay, this is a transcription activation domain) covalently attached through an amide bond to a lactone-cyclized peptide. The lariat peptides are necessary for the yeast two-hybrid assay as this assay requires a transcription activation domain be attached to the cyclic peptide to activate the reporter gene. As discussed below, the yeast two-hybrid assay can be to generate cyclic and lariat peptide affinity agents against a given target. Other activations domains may be utilized, for example, repression domains, split ubiquitin and other two hybrid fusions known in the art, as discussed herein.

Two lariat intein precursor proteins are described, which do not undergo any steps in the cyclization reaction. The first precursor protein, referred to as the unprocessed intein, contains mutations that do not allow any steps to occur in the cyclization reaction. In the unprocessed intein, the combinatorial peptide, genomic fragment, or ScFv is constrained by inserting it between C-intein and N-intein domains. In the unprocessed intein, the activity domain can be attached to either the C-intein or N-intein domain. The second precursor protein, referred to as the dicysteine intein, contains combinatorial peptides, genomic fragments, or ScFvs flanked by cysteines at each end. The dicysteine intein also contains mutations that do not allow the steps in the cyclization reaction to proceed. Combinatorial peptides, ScFvs, or genomic fragments inserted between the C-intein and N-intein domains can be selected that interact with a target molecule. The unprocessed intein or dicysteine intein can be used as affinity agents against a given target. Cyclic peptides based on the sequence of the peptide, ScFv or genomic fragment insert can also be used as affinity agents against a given target. The cysteines at each end of the peptide insert in the dicysteine intein can also be used to cyclize peptide, genomic fragment, or ScFv inserts either through the formation of a disulfide bond or by cross linking the cysteines through a thiol reactive cross linker.

Cyclic peptides are utilized in nature to produce high-affinity drug-like effectors. Both naturally occurring and synthetically designed cyclic peptides have been successfully employed as drugs to treat human diseases (Horswill, A. R. & Benkovic, S. J. (2005) Cell Cycle 4:552-555). Cyclic peptides have an advantage for use as drugs since they have diminished proteolytic susceptibility relative to linear peptides (Humphrey, J. M. & Chamberlin, A. R. (1997) Chem. Rev. 97:2243-2266) and they display enhanced binding to their target due to their restricted conformational space (Horton, D. A. et al. (2002) J. Comput. Aided Mol. Des. 16:415-430, Li, P. & Roller, P. P. (2002) Curr. Top. Med. Chem. 2:325-341), which decreases entropy loss upon binding (Williams et al. (2002) J. Biol. Chem. 277:7790-7798). For these reasons, methods are desired that can rapidly generate cyclic peptides that bind or perturb specific targets.

In one aspect, the invention provides a modified intein library and a method of using the library to screen for cyclic peptides, genomic fragments, and ScFvs, which interact with a specific target or interfere with a specific process. As discussed herein, in some embodimentembodiments, the ‘specific process’ may be protein-protein interactions.

In one embodiment, which can be applied to the lariat intein, unprocessed intein, and dicysteine intein, there is provided a vector which comprises a host-operable promoter operably linked to a nucleic acid molecule comprising, in order, an activity domain, a modified C-intein, an insert, a modified N-intein and a transcription termination sequence.

In another embodiment, which can be applied to the unprocessed intein and dicysteine intein, there is provided a vector which comprises a host-operable promoter operably linked to a nucleic acid molecule comprising, in order, a modified C-intein, an insert, a modified N-intein, an activity domain and a transcription termination sequence.

In some embodiments, the host-operable promoter is a suitable promoter active in the host that is operably linked to the intein library as described herein for driving expression in the host. Examples of such promoters and termination sequences are well-known in the art as are the hosts in which these elements are functional. It is of note that in some embodiments, rather than a strong host-specific promoter, a strong viral promoter, for example, SV40 or CAMV, may be used. As will be appreciated by one of skill in the art, one advantage of these constructs is that they would be functional in multiple hosts. In other embodiments, a tissue-specific promoter or inducible promoter may be used. In a selected embodiment, the host-operable promoter in the vector is a cassette that can be easily replaced using common molecular biology techniques for inserting different expression cassettes or promoter cassettes upstream of the nucleic acid sequence.

As will be apparent to one of skill in the art, the activity domain is selected based on its ability to form a detectable product when in close proximity to a second activity domain. As discussed below, in use, the second activity domain is fused to the target molecule so that interaction between the cyclic peptide, ScFv or genomic fragment encoded by the intein library and the target molecule brings together the two activity domains to produce a detectable product. Examples of suitable activity domains include but are by no means limited to DNA binding domains, transcription activation domains, repression domains, fluorescent proteins and localization sequences, split-ubiquitin, other domains used for protein interaction assays (described above), biotinylation sequence or other antibody epitope tags and protein purification domains such as His tags or GST.

In other embodimentembodiments, the library may be used to screen for disruption or alteration of a specific biological process or cell phenotype. As will be appreciated by one skilled in the art, depending on the nature of the biological process or cell phenotype, positives may be selected based on detecting the disruption of the biological process (as an example, ability to grow on a specific substrate or medium) or cell phenotype.

As will be appreciated by one skilled in the art, interaction of the library member with the target may prevent the target from interacting with another cellular component or may prevent interactions between cellular components other than the target. Thus, in these and similar embodimentembodiments, the library may be used to identify candidates that inhibit protein-protein interactions.

As will be apparent to one of skill in the art, I_{N} and I_{C}refer to the N- and C-intein domains that flank the insert. The modifications made to the intein domains so that the inteins form a lariat, unprocessed intein, or dicysteine intein are discussed below.

In these embodimentembodiments, the insert includes an insertion site for insertion of nucleic acid molecules encoding random peptides, ScFvs, or genomic fragments, as discussed below. As will be appreciated by one of skill in the art, the insertion site may be for example a single restriction site, two adjacent restriction sites or a multiple cloning site as known in the art. For example, the insert may comprise an NruI restriction enzyme recognition site although as will be apparent to one of skill in the art, any suitable restriction enzyme recognition site may be used. It is further noted that ‘suitability’ will be readily understood to one of skill in the art to include factors such as but by no means limited to uniqueness within the vector sequence and enzymatic activity. PCR can also be used to generate a linearized lariat, unprocessed, or dicysteine vector for inserting nucleic acid molecules encoding random peptides, ScFvs, or genomic fragments, as discussed below.

In a further embodiment, there is provided a modified intein lariat library comprising a host-operable promoter operably linked to a nucleic acid molecule comprising, in order, an activity domain, a modified C-intein, an insert having a random peptide, ScFv, or genomic fragment encoding oligonucleotide inserted therein, a modified N-intein and a transcription termination sequence.

In these embodimentembodiments, an oligonucleotide encoding one or more amino acids has been inserted into the insertion site of the insert. As discussed below, the amino acid(s) encoded by the random peptide, ScFv, or genomic fragment encoding oligonucleotide will form the loop of the lariat, unprocessed intein or dicysteine intein.

It is important to note that while generating the oligonucleotide for random peptides or de novo ScFvs that if stop codons are either eliminated or selected against it improves the efficiency of the method in that all or substantially all inserts would form a loop, this is not a necessary feature of the invention. Furthermore, it is important to note that there is no necessary upper or lower limit on the number of amino acids encoded by the oligonucleotide. Yet further, it is important to note that while during preparation of the library, it may be desirable to use oligonucleotides of the same length (encoding for example 6 or 7 or 8 amino acids) to produce for example a 6 amino acid lariat library, in use, these libraries may be combined, as discussed below. It is important to note that while it is certainly desirable that the library contain all combinations of amino acids over a certain length oligonucleotide (for example, for a 5 amino acid lariat, this would be 20×20×20×20×20=3200000 different 5 amino acid lariats) this is by no means a requirement of the invention. Finally, it is important to note that random amino acid libraries do not need to contain all twenty amino acids. Libraries can consists on any combinations of two or more amino acids.

In some embodimentembodiments, the modified intein libraries may be arranged for transformation into a suitable host or may comprise a mixture of host cells already transformed with the library as discussed below.

In use, the modified intein lariat libraries are transformed into a suitable host or host cells or cell line. The cells may be cells that have been previously transformed or transfected with a nucleic acid molecule encoding the target molecule fused to the second activity domain as discussed above. Alternatively, the library may be introduced first and the target may be introduced second or the host may be co-transformed with the library and the target.

Examples of suitable hosts include but are by no means limited to bacteria, yeast, phage, Drosophilia Melanogaster, C. elegans, zebra fish, mice or other model organisms and mammalian cell lines, insect cell lines and the like.

As discussed herein, if a specific modified intein library member interacts with the target molecule, a detectable product is produced and the specific intein library member can be recovered from the host cell expressing the detectable product and sequenced. Examples of such peptides isolated in such a screen are provided below. Accordingly, in one aspect of the invention, there is provided a cyclical peptide identified by the above-described method.

As will be appreciated by one of skill in the art, any molecule that the activity domain can be attached to may be used as a target. It is of note that a large number of protein-protein interactions for a wide variety of peptides have been identified using the yeast two-hybrid system on which this method is based as discussed herein.

As discussed herein, the intein sequences are modified to produce either a lariat structure, which undergoes a partial intein reaction, producing a lariat with a cyclical ‘loop’ and a N-terminal tail to which the activity domain is attached. The unprocessed intein and dicysteine intein do not undergo any steps in the intein reaction and therefore the activity domain can be added to either the C-terminal or N-terminal end. The lariat, unprocessed intein, and dicysteine intein are generated by making specific mutations to the intein sequences thereby blocking complete processing of the intein.

Numbering Scheme for Intein Constructs

A numbering scheme has been developed to assist in comparing heterologous or foreign inteins. This convention numbers the amino acids in inteins sequentially from N-terminal to C-terminal beginning with the first residue of the intein and ending with the last residue of the intein. Split inteins complicate this naming convention and therefore the following numbering scheme is used: (i) The I_{N} intein domain is numbered {I_{N+1}, I_{N+2}, I_{N+3} . . . } from N-terminus to C-terminus; (ii) The extein is numbered from the C-terminus to the N-terminus {I_{N-1}, I_{N-2}, I_{N-3} . . . } or from the N-terminus to the C-terminus (I_{C+1}, I_{C+2}, I_{C+3} . . . }; (ii) The I_{C} intein domain is then {I_{C-1}, I_{C-2}, I_{C-3} . . . } from the C-terminus to the N-terminus of the intein (Perler, F. B. (2002). Nucl. Acids Res. 30:383-384).

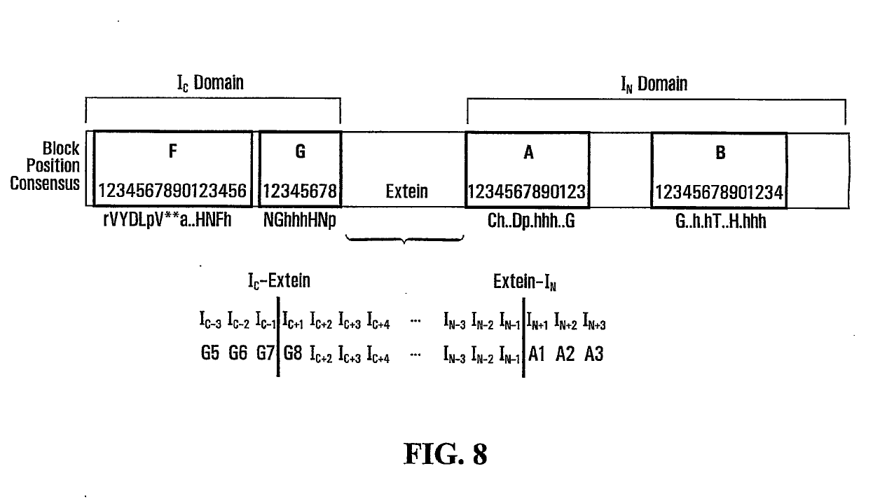

This numbering system makes it difficult to compare conserved amino acids sites that are not close to the splice site between different inteins. To facilitate referring to these conserved amino acids, the present disclosure sets out a naming scheme based on conserved intein motifs. Several conserved motifs have been observed by comparing intein amino acid sequences. There are two nomenclatures for these motifs: (i) Blocks A, B, C, D, E, H, F, G (Pietrokovski, S. (1994) Protein Sci 3:2340-50, Telenti, A. et al. (1997) J. Bacteriol. 179:6378-82) and (ii) Blocks N1, N3, EN1, EN2, EN3, EN4, C2 and C1 (Pietrokovski, S. (1998) Protein Sci. 7:64-71). The present disclosure uses the A, B, C, D, E, H, F, G nomenclature and assigns each amino acid position in a conserved block a number from N-terminus to C-terminus. For example, which is the eighth amino acid from the N-terminus in block G, is labelled G8. Similarly, I_{NA}, which is the first amino acid from the N-terminus of block A, is labelled A1. The I_{N} intein domain contains blocks A and B and the I_{C}intein domain contains blocks F and G. The region to be cyclized or the extein is numbered from N-terminus to C-terminus {I_{C+1}, I_{C+2}, I_{C+3}, . . . , I_{N-3}, I_{N-2}, N_{N-1}} (See FIG. 8 for overview of numbering scheme).

In this disclosure, amino acids within 5 amino acids of the splice junctions will be named using both conventions i.e. I_{C+1} (G8). Amino acids further than 5 amino acids from the splice site will be referred to by their conserved block and amino acid number.

Mechanisms of Intein-Mediated Protein Splicing

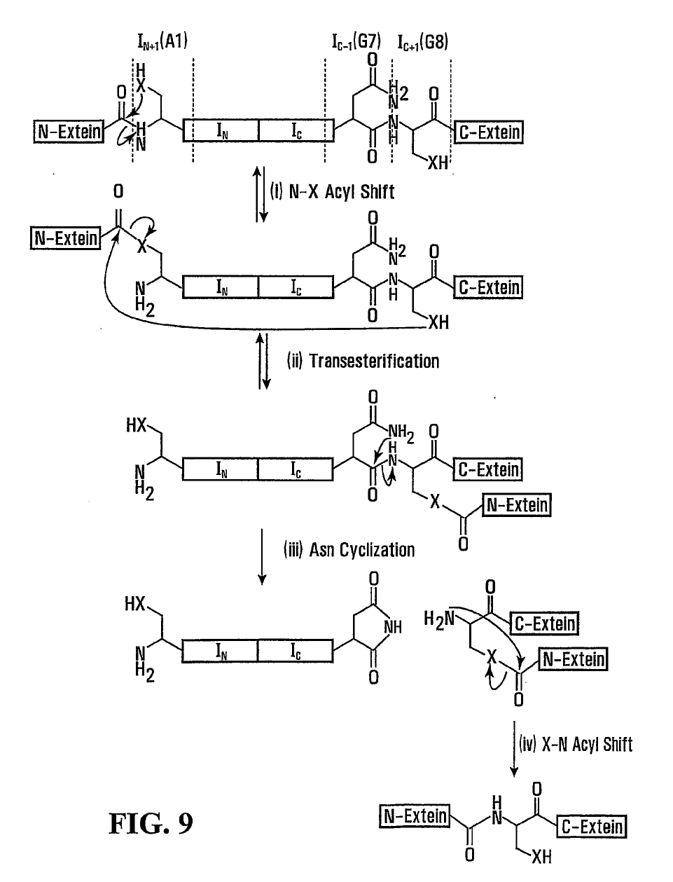

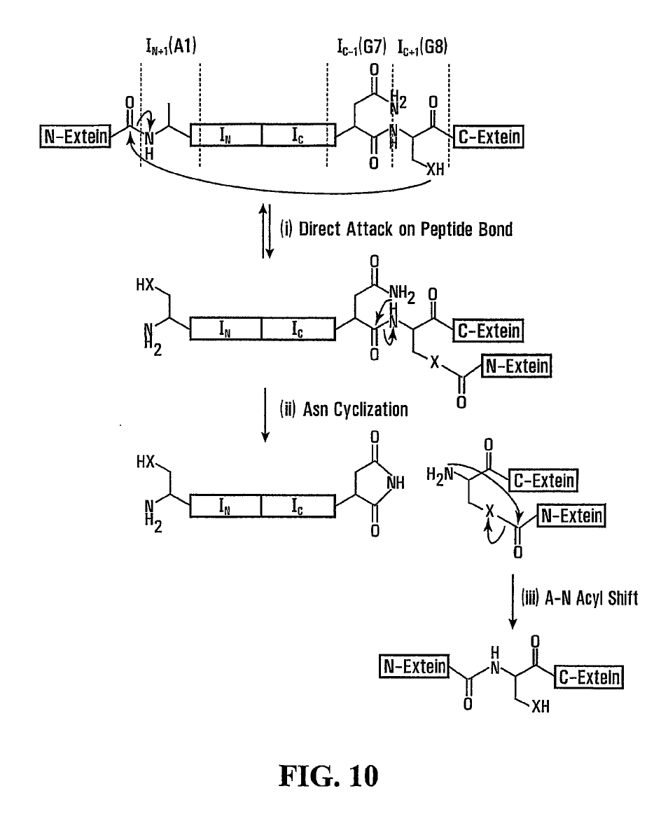

There are two proposed mechanisms for intein-mediated protein splicing. The first mechanism is the most common and will be referred to as the “standard” mechanism (FIG. 9). The second less common mechanism will be referred to as the “non-standard” mechanism (FIG. 10). Step 1 in the standard mechanism involves an N—X acyl shift (where X=Cys or Ser) at position I_{N+1} (A1). The acyl shift introduces a thioester or an ester into the amide backbone of the peptide. Ester bonds are more labile than amide bonds and thus provide a good leaving group for the reaction in Step 2 (Transesterification reaction). Formation of the ester bond also positions the ester bond for attack by the I_{C+1} (G8) Ser or Cys nucleophile in Step 2 (Transesterification reaction) (Southworth, M. W. et al. (2000) EMBO J. 19:5019-5026, Poland, B. W. et al. (2000) J. Biol. Chem. 275:16408-16413). In

Step 2 (Transesterification reaction), either Cys, Ser, or Thr at position I_{C+1} (G8) can act as a nucleophile that reacts with the thioester or ester bond formed in the Step 1 (N—X acyl shift). This results in the cleavage of the I_{N} domain from the intein between amino acids at position I_{N+1} (A1) and I_{N-1} and the formation of a branched intermediate via a thioester or ester bond between I_{C+1} (G8) and I_{N-1}. In Step 3, Asn cyclization cleaves the amide bond that connects amino acids at positions I_{C+1} (G8) and I_{C-1} (G7) and releases the extein, which contains a thioester or ester bond between I_{C+1} (G8) and I_{N-1}). Gln also occurs at position I_{C-1} (G7) and undergoes a similar cyclization reaction (Pietrokovski, S. (1998) Protein Sci. 7:64-71). The last step, a lactone to lactam transformation, converts the ester bond between I_{C+1} (G8) and I_{N-1} in the extein to an amide bond. Based on the 344 intein sequences in the InBase database (Perler, F. B. (2002). Nucl. Acids Res. 30:383-384) the following amino acids occur at sites described above: Cys (281/344) and Ser (34/344) occur at position I_{N+1} (A1); Cys (139/344), Ser (120/344), and Thr (81/344) occur at position I_{C+1} (G8); and Asn (327/344) and Gln (15/344) occur at position I_{C-1} (G7).

In the non-standard mechanism there is no N—X acyl shift (Step1 in standard mechanism). For inteins that use the non-standard mechanism, Ser or Cys at position I_{N+1} (A1) is replaced by Ala. Ala occurs at I_{N+1} (A1) in 25/344 inteins in InBase (Perler, F. B. (2002). Nucl. Acids Res. 30:383-384). Inteins that have Ala at position I_{N+1} (A1) undergo a direct nucleophilic attack on the peptide backbone between I_{N-1} and I_{N+1} (A1) using the amino acid at position I_{C+1} (G8) (FIG. 10) (Southworth, M. W. et al. (2000) EMBO J. 19:5019-5026). Step 1 (N—X acyl shift) is not needed in inteins that use the non-standard mechanism since the amide bond is already aligned for direct attack by the nucleophile at I_{C+1} (G8) and therefore they do not need the extension in the backbone caused by Step 1 (N—X acyl shift) (Southworth, M. W. et al. (2000) EMBO J. 19:5019-5026, Poland, B. W. et al. (2000) J. Biol. Chem. 275:16408-16413).

Other amino acids have also been observed substituted at the three positions that are directly involved in splicing (I_{C+1} (G8), I_{C-1} (G7), and I_{N+1} (A1)). These inteins may also use a mechanism for intein splicing that is different from the standard mechanism. For example, Asp has been identified at I_{C-1} (G7) in place of Asn (1/344) (Amitai, G. et al. (2004) J. Biol. Chem. 279:3121-3131). This intein may undergo Asp cyclization at Step 3 (Asn cyclization) of the standard mechanism. This intein however, is still capable of splicing even if Asp is mutated to Ala, which indicates that there are yet undetermined non-standard mechanisms for intein splicing (Amitai, G. et al. (2004) J. Biol. Chem. 279:3121-3131).

Several other inteins have been identified that also have other amino acids at the three amino acids directly involved in splicing (I_{C+1} (G8), I_{C-1} (G7), I_{N+1} (A1)). For these inteins, there is no information on their mechanism(s) of splicing or if they are capable of splicing. Some examples of these inteins that contain other amino acids at indicated sites include: Gln (2/344) (Cth TerA and PhiEL ORF11 inteins), Met (1/344) (PhiEL ORF40 intein), and Pro (1/344) (Mbe DnaB intein) at position I_{N+1} (A1); Val (2/344) (Cth ATpase and Pfi Fha), Gly (1/344) (Avin RIR1), and Tyr (1/344) (Mmag Magn8951) at position I_{C+1} (G8); and His (1/344) (Mga SufB (Mga Pps1)) at position I_{C-1} (G7).

Describing Intermediate Structures in the Intein Reaction

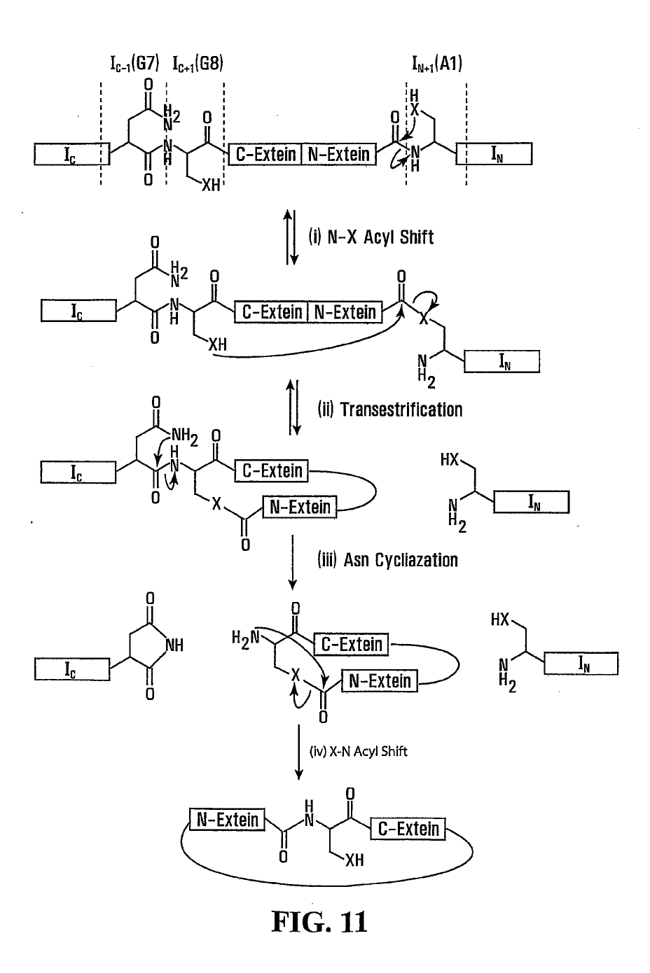

Understanding the intein-mediated splicing mechanism allows us to interrupt splicing at different points in the mechanism and isolate mutant inteins that are useful in many biological applications. The mutant inteins described in this invention refer to mutants in the intein-mediated protein cyclization reaction (FIG. 11). These mutants are referred to as the unprocessed intein, the dicysteine intein, and the lariat intein. These three mutant inteins are described by the mutations required to generate them.

To generate the unprocessed intein, Step 2 (Transesterification reaction) needs to be inhibited. The transesterification reaction releases the I_{N} domain. If the I_{N} domain is released, then the I_{C}-I_{N} domain interaction responsible for the scaffolding ability of this mutant is disrupted. To further stabilize the unprocessed intein, Step 1 (N—X acyl shift) should also be inhibited. Step 1 (N—X acyl shift) results in the formation of a thioester or ester bond at the Extein-I_{N} junction, between I_{N-1} and I_{N+1} (A1). The thioester or ester bond is more susceptible to hydrolysis than an amide bond. Hydrolysis results in cleavage at the Extein-I_{N} junction and release of the I_{N} domain. Cleavage at the I_{C}-Extein junction between I_{C-1} (G7) and I_{C+1} (G8) also occurs at a slow rate due to Asn cyclization (Step 3) (Xu, M. & Perler, F. B. (1996) EMBO J. 15:5146-5153). To stabilize the intein from I_{C}-Extein cleavage by Asn cyclization, Step 3 (Asn cyclization) should also be inhibited. Inhibition of all three steps in the intein reaction (N—X acyl shift, Transesterification reaction, and Asn cyclization) results in the most stable unprocessed intein (Xu, M. & Perler, F. B. (1996) EMBO J. 15:5146-5153).

Dicysteine inteins have Cys at positions I_{C+1} (G8) and I_{N+1} (A1) that are used to cross link peptides, genomic fragments, or ScFvs that interact with a target. Since these amino acids can function as nucleophiles in Step 1 (N—X acyl shift) and Step 2 (Transesterification reaction) of the intein reaction, strategies are needed that inhibit these steps without mutating these Cys. At minimum, Step 2 (Transesterification reaction) needs to be inhibited to prevent formation of an unstable thioester bond between I_{C+1} (G8) and I_{N-1} the last residue of the extein, which results in the cleavage of the I_{N} domain. Similar to the unprocessed intein, the dicysteine intein can be stabilized by inhibiting Step 1 (N—X acyl shift), which prevents the hydrolysis of the Extein-I_{N} ester or thioester. The dicysteine intein can be further stabilized by inhibiting Step 3 (Asn cyclization), which prevents I_{C}-Extein cleavage between I_{C-1} (G7) and I_{C+1} (G8).

The lariat intein is generated by inhibiting the Step 3 (Asn cyclization) in the intein reaction. The lariat intein is cyclized through a lactone bond, which is more susceptible to hydrolysis than an amide bond. The lariat can be further stabilized by inhibiting hydrolysis of the lactone bond.

Overview of Methods to Inhibit Steps in the intein Reaction.

The strategies, as described below, can be used either alone or in combinations to generate unprocessed inteins, dicysteine inteins, or lariat inteins.

Step 1: N—X Acyl Shift

The N—X acyl shift involves the I_{N+1} (A1) nucleophile, which is usually Ser or Cys. Although Thr is not normally present at position I_{N+1} (A1), it could also potentially function as a nucleophile in Step 1 (N—X acyl shift). Step 1 (N—X acyl shift) produces an ester or thioester bond that replaces the amide bond between the I_{N+1} (A1) residue and the I_{N-1} residue (the last residue of the extein). The ester or thioester forms a good leaving group for Step 2 (Transesterification), however the ester or thioester bond is susceptible to hydrolysis, which can result in cleavage between the Extein-I_{N} at I_{N-1} and I_{N+1} (A1). Therefore, if Step 2 (Transesterification) is inhibited, I_{N} cleavage by hydrolysis can become a side product. Mutation of amino acids that are involved in catalyzing the N—X acyl shift can block Step 1 in the intein reaction. The catalytic pocket where the N—X acyl bond is formed contains amino acids in Block B: B7 (Thr69^{Ssp DnaE}, Thr70^{Ssp DnaB}), B9 (Asn72^{Ssp DnaB}), B10 (His72^{Ssp DnaE}, His73^{Ssp DnaB}), amino acids in Block F: F2 (Val134^{Ssp DnaB}), F3 (Phe139^{Ssp DnaE}), F4 (Asp140^{Ssp DnaE}), amino acids between Blocks A and B: Arg50^{Ssp DnaE}, Thr51^{Ssp DnaB}, Lys54^{Ssp DnaB}, the nucleophile in Block A: A1 (Cys1^{Ssp DnaE}), the adjacent amino acid in Block A: A2 (Leu2^{Ssp DnaE}), and the last residue of the extein: I_{N-1} (Sun, P. et al. (2005) J. Mol. Biol. 353:1093-1105, Ding, Y. et al. (2003) J. Biol. Chem. 278:39133-39142).

The following strategies can be use to inhibit Step 1 (N—X acyl shift) in the intein reaction. These strategies can be used to generate the unprocessed intein and the dicysteine intein.

Strategy 1.1: Mutation of the I_{N+1} (A1) nucleophile. Mutation of the I_{N+1} (A1) to a non-nucleophillic amino acid prevents the formation of the ester or thioester (Ding, Y. et al. (2003) J. Biol. Chem. 278:39133-39142, Sun, P. et al. (2005) J. Mol. Biol. 353:1093-1105, Xu, M. & Perler, F. B. (1996) EMBO J. 15:5146-5153). Mutation of Ser at position I_{N+1} (A1) to Ala prevents Step 1 (N—X acyl shift) in Psp Pol-I intein (Xu, M. & Perler, F. B. (1996) EMBO J. 15:514.6-5153). Mutation of Cys at position I_{N+1} (A1) to Arg, Gly, or Val inhibits Step 1 (N—X acyl shift) in the Sce VMA intein (Cooper, A. A. et al. (1993) EMBO J. 12:2575-2583). Many inteins are inhibited when the nucleophile at position I_{N+1} is mutated to another nucleophilic amino acid. Some examples include the Psp Pol-I intein that splices poorly when Ser I_{N+1} (A1) is mutated to Cys, and is blocked completely when Ser is mutated to Thr (Xu, M. & Perler, F. B. (1996) EMBO J. 15:5146-5153). Similarly, the Sce VMA1 intein does not tolerate a mutation of Cys at position I_{N+1} (A1) to Ser (Hirata, R. & Anraku, Y. (1992) Biochem. Biophys. Res. Commun. 188:40-47, Cooper, A. A. et al. (1993) EMBO J. 12:2575-2583). Strategy 1.1 is useful for inhibiting Step 1 (N—X acyl shift) in inteins that use the standard mechanism.

Strategy 1.2: Mutation of the F3 amino acid. Analysis of Ssp DnaE, PI Scel, and Ssp DnaB intein structures reveals that amino acids at position F3 are in the catalytic pocket where the N—X acyl bond is formed. Mutation of Phe at position F3 in Ssp DnaE to Ala inhibits the formation of the ester or thioester between I_{N-1} and I_{N+1} (Ghosh, I. et al. (2001) J. Biol. Chem. 276:24051-24058).

Strategy 1.3: Mutation of amino acids within hydrogen bonding distance of I_{N+1} (A1). Analysis of Ssp DnaE, PI Scel, and Ssp DnaB intein structures reveals that Arg50 in Ssp DnaE (Sun, P. et al. (2005) J. Mol. Biol. 353:1093-1105) and Thr51 in Ssp DnaB (Ding, Y. et al. (2003) J. Biol. Chem. 278:39133-39142) interact with the I_{N+1} (A1) nucleophile. In accordance with alternative embodiments, mutations of of amino acids within hydrogen bonding distance of I_{N+1} (A1) may be used to disrupt Step 1 (N—X acyl shift) or Step 2 (Transesterification reaction). In alternative aspects, mutations that block Step 1 may accordingly include substitutions at positions B9, B10, or F2 (the equivalent amino acids to Arg50 SspDnaE and Thr51 in SspDnaB), including substitution of non-catalytic amino acids at these positions.

Step 2: Transesterification Reaction

The transesterification reaction involves nucleophilic amino acids at position I_{C+1} (G8) attacking the ester or thioester bond formed in Step 1 (N—X acyl shift), which results in the formation of a ester or thioester bond between I_{C+1} (GB) and I_{N-1}. The transesterification reaction releases the I_{N} domain from the I_{C}-extein domain (Split intein product). The ester or thioester bond formed between the I_{C+1} (08) residue and I_{N-1} can potentially be hydrolysed resulting in a linear intein product consisting of I_{C}-extein (Split intein product). I_{C+2} and I_{N-1} are found in the catalytic pocket for the transesterification reaction and can potentially influence splicing.

The following strategies can be use to inhibit Step 2 (Transesterification reaction) in the intein reaction. These strategies can be used to generate the unprocessed intein and the dicysteine intein.

Strategy 2.1: Mutation of the I_{C+1} (G8) nucleophile. Amino acid at position I_{C+1} (G8) functions as a nucleophile in the transesterification reaction. Mutations of nucleophilic amino acids at position I_{C+1} (G8) inhibit transesterification. For example the following mutations at position I_{C+1} (G8) block the transesterification reaction: Ser to Ala in the Psp pol intein (Xu, M. & Perler, F. B. (1996) EMBO J. 15:5146-5153); Cys to Ala in the Mja Klb intein (Southworth, M. W. et al. (2000) EMBO J. 19:5019-5026); and Cys to Arg, Gly, or Val in the Sce Tfp1 intein (Cooper, A. A. et al. (1993) EMBO J. 12:2575-2583). Asn cyclization is severely inhibited in vitro when I_{C+1} (G8) Cys is mutated to Pro; moderate to severely inhibited by Val, Ile, Asp, Glu, Lys, Arg, and His, moderately inhibited by Gly, Leu, Asn, Trp, Phe, and Tyr; and minimally inhibited by Met, Ala, Gln in the Sce VMA intein (New England Biolabs, IMPACT™-CN protein purification system).

Mutation of Cys at I_{C+1} (G8) to Ser inhibits transesterification, but stabilizes the branched intermediate in the Sce VMA intein (Chong, S. et al. (1996) J. Biol. Chem. 271:22159-22168, Cooper, A. A. et al. (1993) EMBO J. 12:2575-2583). Certain inteins are unable to function using other nucleophilic amino acids at position I_{C+1} (G8) (Shingledecker, K. et al. (2000) Archives Biochem. Biophys. 375:138-144). For example, in Psp Pol-I intein, Step 2 (Transesterification reaction) is inhibited, when Ser I_{C+1} (G8) is mutated to Cys or Thr, (Xu, M. & Perler, F. B. (1996) EMBO J. 15:5146-5153). Similarly, mutation of Cys at position I_{C+1} (G8) to Ser inhibits Step 2 (Transesterification reaction) in the Sce VMA1 intein (Hirata, R. & Anraku, Y. (1992) Biochem. Biophys. Res. Commun. 188:40-47).

Strategy 2.2: Mutation of the B7 amino acid. Analysis of the Ssp DnaE, PI Scel, and Ssp DnaB intein structures suggests that amino acids at position B7 are involved in Step 2 (Transesterification reaction). Amino acids at position B7 (Thr69^{Ssp DnaE} (Sun, P. et al. (2005) J. Mol. Biol. 353:1093-1105), Thr73^{Ssp DnaE} (Ding, Y. et al. (2003) J. Biol. Chem. 278:39133-39142), and Asp76^{PI Scel} (Werner, E. et al. (2002) Nucl. Acid Res. 30:3962-3971)) stabilize the carbonyl oxygen of Cys at position I_{N+1}(A1). Mutational studies confirm the role of B7 in Step 2 (Transesterification reaction). Mutation of Thr at position B7 to Ala inhibits the transesterification reaction in the Ssp DnaE intein; I_{N} cleavage can be induced in vitro by DTT, which cleaves ester or thioester bonds, demonstrating that Step 1 (N—X acyl shift) and possibly Step 2 (Transesterification reaction) occurs with this mutation (Ghosh, I. et al. (2001) J. Biol. Chem. 276:24051-24058). Further experiments with Mja KlbA intein, which uses a non-standard mechanism, show the mutation of Thr at position B7 to Ala inhibits ester or thioester cleavage in the presence of DTT (Southworth, M. W. et al. (2000) EMBO J. 19:5019-5026). In the Mja KlbA intein, the I_{C+1} nucleophile directly attacks the amide bond between I_{N-1} and I_{N+1} (A1), the only bond susceptible to DTT is the ester or thioester in the branched intermediate formed by Step 2 (Transesterification reaction). This indicates that Step 2 (Transesterification reaction) is inhibited by mutation at position B7.

Strategy 2.3: Mutation of the B10 amino acid. Analysis of the Ssp DnaE, PI Scel, and Ssp DnaB intein structures implicates B10 in I_{N}-Extein splicing. Amino acids at position B10 (His72^{Ssp DnaE} (Sun, P. et al. (2005) J. Mol. Biol. 353:1093-1105), His73^{Ssp DnaB} (Ding, Y. et al. (2003) J. Biol. Chem. 278:39133-39142), and His79^{PI Scel} (Werner, E. et al. (2002) Nucl. Acid Res. 30:3962-3971)) hydrogen bond with the amido nitrogen of Cys1^{Ssp DnaE} at position A1 (I_{N+1}). Mutational studies confirm its role in the transesterification reaction. Mutation of His at position B10 to Ala prevents splicing in the Ssp DnaE intein, but I_{N} cleavage can be induced in vitro using DTT, which cleaves ester or thioester bonds, demonstrating that Step 1 (N—X acyl shift) occurs with mutations at B10 (Ghosh, I. et al. (2001) J. Biol. Chem. 276:24051-24058). Further experiments with Mja KlbA intein, which uses a non-standard mechanism, show the mutation of His at position B10 to Ala inhibits ester or thioester cleavage in the presence of DTT (Southworth, M. W. et al. (2000) EMBO J. 19:5019-5026). In the Mja KlbA intein, the I_{C+1} nucleophile directly attacks the amide bond between I_{N-1} and I_{N+1} (A1), the only bond susceptible to DTT is the ester or thioester in the branched intermediate formed by Step 2 (Transesterification reaction). This indicates that Step 2 (Transesterification reaction) is inhibited by mutation at position B10.

Strategy 2.4: Introduction of a charged amino acid near the splice sites. Mutation of Leu at position I_{N+2} (A2) in Psp Pol or mutation of Ala at position I_{C-2} (G6) in Psp Pol to Lys prevents cleavage of the I_{N} domain (Xu, M. & Perler, F. B. (1996) EMBO J. 15:5146-5153). Mutation of Val at position I_{C-2} (G6) to Arg or Phe blocks splicing in the Sce VMA intein, however, Ser, Cys, Ile, and Gly mutations do not inhibit splicing (Cooper, A. A. et al. (1993) EMBO J. 12:2575-2583). Mutations that introduce a charge at positions I_{N+2} (A2) and I_{C-2} (G6) should inhibit the transesterification reaction.

Strategy 2.5: Mutation of the F4 amino acid. Analysis of intein structures reveals that the amino acid at position F4 (Asp140^{Ssp DnaE}) hydrogen bonds the carbonyl oxygen of the I_{N-1} (Tyr-1^{Ssp DnaE}) amino acid (Sun, P. et al. (2005) J. Mol. Biol. 353:1093-1105). Mutation of Asp140 to Ala in the Ssp DnaE intein prevents splicing (Ghosh, I. et al. (2001) J. Biol. Chem. 276:24051-24058).

Strategy 2.6: Zinc-mediated inhibition. Zinc coordinates with the I_{C+1} (G8) nucleophile and prevents splicing (Mills, K. V. & Paulus, H. (2001) J. Biol. Chem. 276:10832-10838). Addition of Zinc at concentrations greater than 10 μM should block the transesterification reaction.

Strategy 2.7: The amino acid at position F6 coordinates the Ser (G8) for attack on the thioester formed in Step1. Accordingly, mutation at position F6 to a non-catalytic residue may be used to block Step 2.

Step 3: Asn Cyclization: Step 3 (Asn cyclization) results in cleavage of the I_{C}domain from the extein. The most common mechanism for this step involves Asn cyclization. This mechanism is used by 327 of the 344 inteins in the InBase database. The second most common method involves Gln cyclization, which is used by 15 of the 344 inteins in the InBase database. The following amino acids are important in forming the catalytic pocket for Asn cyclization: Block B: B11 (Arg73^{Ssp DnaE}); Block F: F5 (Leu137^{Ssp DnaB}), F6 (Thr138^{Ssp DnaB}), F7 (Val139^{Ssp DnaB}, Leu143^{Ssp DnaE}), F13 (His143^{Ssp DnaB}, His147^{Ssp DnaE}); Block G: G6 (His153^{Ssp DnaB}), G7 (Asn154^{Ssp DnaB}, Asn159^{Ssp DnaE}), G8 (Ser155^{Ssp DnaE}, Cys160^{Ssp DnaE}), the second residue of the extein (I_{C+2}), and the last residue of the extein (I_{N-1}) (Sun, P. et al. (2005) J. Mol. Biol. 353:1093-1105, Ding, Y. et al. (2003) J. Biol. Chem. 278:39133-39142).

To generate lariat, unprocessed, and dicysteine inteins, mutations are needed that block Asn cyclization. The following strategies have been developed to inhibit Asn cyclization.

Strategy 3.1: Mutation of amino acids at position I_{C-1} (G7). Mutation of amino acids at position I_{C-1} (G7) to any non-native amino acid inhibits Step 3 (Asn cyclization). Mutation of Asn at position I_{C-1} (G7) to Gln and Asp may not block Step 3 (Asn cyclization) (Amitai, G. et al. (2004) J. Biol. Chem. 279:3121-3131) and therefore should be avoided. Mutation of Asn at position I_{C-1} (G7) in the Sce Tfp1 intein to Lys, Ala, Tyr, Gln, Glu, His, and Asp all inhibit Step 3 (Asn cyclization) (Cooper, A. A. et al. (1993) EMBO J. 12:2575-2583). In addition to inhibiting Step 3 (Asn cyclization), mutation of Asn at position I_{C-1} (G7) to hydrophobic amino acids may also stabilize the ester or thioester formed in Step 2 (Transesterification reaction). This prediction is based on the observed accumulation of branched intermediate when His at position I_{C-2} (G6) is mutated to a Leu, Asn, or Gln (Xu, M. & Perler, F. B. (1996) EMBO J. 15:5146-5153). Therefore, certain mutations of Asn at position I_{C-1} (G7) may stabilize the lariat.

This prediction is further supported by the observation that the branched intermediate accumulates when Asn at position I_{C-1} (G7) is mutated in the Sce VMA intein. Mutation of Asn at position I_{C-1} (G7) to Ser or Ala (Chong, S. et al. (1996) J. Biol. Chem. 271:22159-22168) does not result in the accumulation of the branched intermediate, however mutation of Asn at position I_{C-1} (G7) to Lys results in an accumulation of branched intermediate (Kawasaki M. et al. (1997) J. Biol. Chem. 272:15668-15674). Mutation of both Asn at position I_{C-1} (G7) to Ala and Cys at position I_{C+1} (G8) to Ser in the Sce VMA intein results in an accumulation of branched intermediate (Chong, S. et al. (1996) J. Biol. Chem. 271:22159-22168). The environment surrounding the ester or thioester bond formed in the transesterification reaction appears to plays a role in stabilizing the branched intermediate.

Strategy 3.2: Mutation of amino acids at positions G6 (I_{C-2}) or B11 that hydrogen bond with the Asn carbonyl oxygen at position I_{C-1} (G7). His at position I_{C-2} (G6) assists in Asn cyclization (Xu, M. & Perler, F. B. (1996) EMBO J. 15:5146-5153) by hydrogen bonding to the Asn carbonyl oxygen at position I_{C-1} (G7), making this peptide bond more labile (Klabunde, T. et al. (1998) Nat. Struct. Biol. 5:31-36, Duan, X. et al. (1997) Cell 89:555-564). However, in the Ssp DnaE and other inteins that have Ala at position I_{C--2} (G6) instead of His, there are conflicting reports on the role of position I_{C-2} (G6) in Step 3 (Asn cyclization). Structural analysis of the Ssp DnaE (Sun, P. et al. (2005) J. Mol. Biol. 353:1093-1105) and Ssp DnaB (Ding, Y. et al. (2003) J. Biol. Chem. 278:39133-39142) inteins has provided insight to the role of amino acids at position I_{C-2} (G6). In the Ssp DnaB Intein, His at position I_{C-2} (G6) binds to the Asn carbonyl oxygen at position I_{C-1} (G7). In the Ssp DnaE intein, Arg at position B11 binds to the Asn carbonyl oxygen at position I_{C-1} (G7) (Ding, Y. et al. (2003) J. Biol. Chem. 278:39133-39142). The use of His or Arg to interact with the Asn carbonyl oxygen depends on residues in the extein. Phe at position (I_{C+2}) and Phe at position (I_{N-4}) in the extein form a hydrophobic pocket that interacts with the imidazole ring of His at position I_{C-2} (G6), which prevents it from interacting with the Asn carbonyl oxygen at position I_{C}₋₁ (G7) (Sun, P. et al. (2005) J. Mol. Biol. 353:1093-1105). Mutation of His at position I_{C-2} (G6) in the Psp pol-I intein to Leu, Asn, and Gln results in an accumulation of the branched intermediate (Xu, M. & Perler, F. B. (1996) EMBO J. 15:5146-5153). Mutation of His I_{C-2} (G6) to Gin prevents Asn cyclization in the Sce VMA intein (New England Biolabs, IMPACT™-CN Protein purification system). However when His at position I_{C-2} (G6) is mutated to Leu and Asn at position I_{C-1} (G7) is mutated to Ala no branched intermediate accumulates, suggesting that Asn is important for branched intermediate accumulation. Currently, there are no mutagenic studies on the role of Arg at position B11 in accumulating branched intermediates.

Strategy 3.3: Mutation of the amino acids at position F13. Mutation of His at position F13 in the Ssp DnaB intein to Gln blocks Step 3 (Asn cyclization) (Ding, Y. et al. (2003) J. Biol. Chem. 278:39133-39142). Mutation of His at position F13 in the Ssp DnaB intein to Ala only partially inhibits Step 3 (Asn cyclization) (Ding, Y. et al. (2003) J. Biol. Chem. 278:39133-39142).

Strategy 3.4: Mutation of the amino acid at position F14. Mutation of Asn at position F14 in the Ssp DnaE intein to Ala inhibits Asn cyclization (Ghosh, I. et al. (2001) J. Biol. Chem. 276:24051-24058). However, Mutation of Asn at position F14 in the Ssp DnaB intein to Ala has no effect on Step 3 (Asn cyclization) (Ding, Y. et al. (2003) J. Biol. Chem. 278:39133-39142).

Strategy 3.5: Mutation of the amino acids at position F15. The amino acid at position F15 is highly conserved. Mutation of Phe at position F15 in the Ssp DnaB intein to Ala blocks Step 3 (Asn cyclization) (Ding, Y. et al. (2003) J. Biol. Chem. 278:39133-39142). Mutation of Phe at position F15 in the Ssp DnaB intein to Tyr slightly inhibits Step 3 (Asn cyclization) (Ding, Y. et al. (2003) J. Biol. Chem. 278:39133-39142).

Strategy 4: Mutations in the extein region. Amino acids in the extein located near the splice site effect splicing. Evans et al., provided evidence that the I_{N-1}, I_{N-2} amino acids at the Extein-I_{N} junction, and I_{C+2}, I_{C+3}, I_{C+4} amino acids at the I_{C}-Extein junction are required for splicing in the Ssp DnaE intein (Evans, T. C. Jr. et al. (2000) J. Biol. Chem. 275:9091-9094). In the Ssp DnaE intein, the amino acid at position I_{C+2} in the extein is involved in intein-mediated splicing (Iwai, H. et al. (2006) FEBS Lett. 580:1853-1858). Mutation of Phe at position I_{C+2} in Ssp DnaE intein to an amino acid other than Phe, Tyr, or Trp inhibits intein-mediated splicing (Iwai, H. et al. (2006) FEBS Lett. 580:1853-1858). A mixed intein containing the I_{N} domain from Npu DnaE intein and an I_{C}domain from Ssp DnaE intein is much more tolerant to amino acid substitutions at this position (Iwai, H. et al. (2006) FEBS Lett. 580:1853-1858). Therefore fixing this amino acid in random libraries may be beneficial when using certain inteins. Amino acids at position I_{N-1} are found in the N—X acyl shift catalytic pocket (Sun, P. et al. (2005) J. Mol. Biol. 353:1093-1105). In Ssp DnaE, Tyr at position I_{N-1} has been proposed to act as a switch that prevents Step 3 (Asn cyclization) from occurring before Step 2 (Transesterification reaction) is finished (Sun, P. et al. (2005) J. Mol. Biol. 353:1093-1105). A modified Sce VMA intein used for protein purification, is fused C-terminal to the target protein, the Sce VMA is mutated to prevent Step 3 (Asn Cyclization) and Step 2 (Transesterification reaction) allowing only Step 1 (N—X acyl shift) to occur. Certain amino acids at position I_{N-1} in Sce VMA intein allow Step 1 (N—X acyl shift) to occur in vivo: Thr, Glu, His, Arg, and Asp. The following amino acids at position I_{N-1} in Sce VMA intein inhibit Step 1 (N—X acyl shift) in vivo but not in vitro: Gly, Ala, Ile, Leu, Met, Phe, Val, Gin, Ser, Trp, Tyr, and Lys. The following amino acids at position I_{N}₋₁ prevent in Sce VMA intein inhibit Step 1 (N—X acyl shift) in vivo and in vitro: Asn, Cys, and Pro (New England Biolabs, IMPACT™-CN Protein purification system). Fixing the extein amino acids near the slice junctions is a useful strategy for generating lariat inteins.

Description of Inteins

Description of Unprocessed Intein

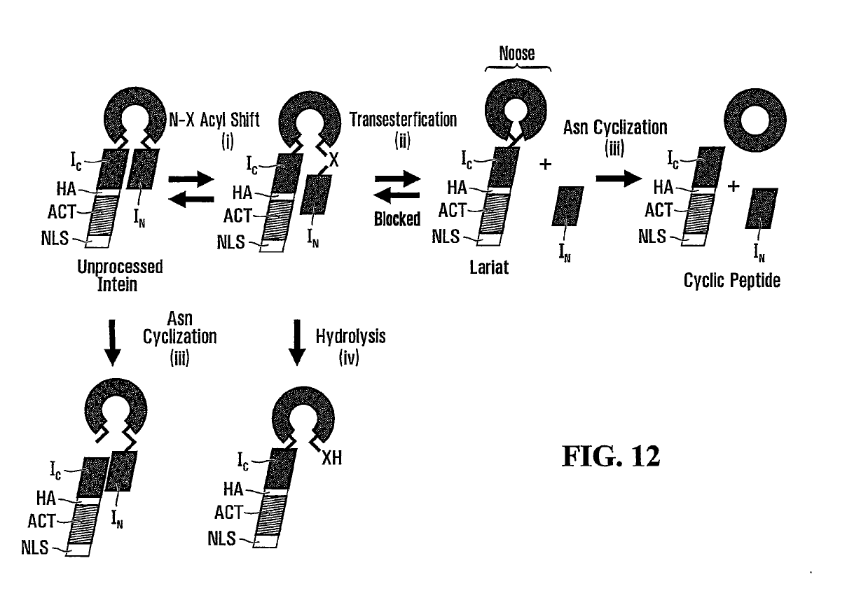

In the unprocessed intein, no cyclized peptide or “noose” is formed. The I_{N} and I_{C}domains fold to display the peptide, ScFv, or genomic fragment in a constrained, cyclic-like conformation (FIG. 12). The most important mutations for constructing the unprocessed intein are mutations that inhibit Step 2 (Transesterification reaction) (FIG. 12). When only Step 2 (Transesterification reaction) is inhibited, it is still possible for unprocessed intein to undergo Step 1 (N—X acyl shift) and Step 3 (Asn cyclization) (FIG. 12). If Step 1 (N—X acyl shift) occurs then the amino acids at I_{N+1} (A1) and I_{N-1} that form the Extein-I_{N} boundary will be linked via an ester or thioester bond. This bond can undergo hydrolysis more rapidly than an amide bond, which would result in the release of the I_{N} domain. If Step 3 (Asn cyclization) occurs then Asn cyclization still occurs at a slow rate, which results in Extein-I_{C} cleavage. To stabilize the unprocessed intein, Step 1 (N—X acyl shift) and Step 3 (Asn cyclization) need to be inhibited.

For each of the strategies described, there may be more than one amino acid substitution that will work. For example, in Strategy 2.1, Step 2 (Transesterification) may be blocked by mutating Ser at position I_{C+1} (G8) to Ala. It may also be blocked by mutating Ser at position I_{C+1} (G8) to other amino acids. When a strategy refers to a mutation, there may be multiple amino acid substitutions at that site that will accomplish the same outcome.

Generating the Unprocessed Intein by Inhibiting Step 2 (Transesterification reaction) Unprocessed intein can be generated using a single strategy or a combination of strategies that inhibit only Step 2 (Transesterification reaction). If I_{C+1} (G8) is not Cys, Strategy 2.6 will have no effect, leaving strategies (2.1-2.5), which results in a total of 2⁵−1=31 strategies for inhibiting Step 2. If I_{C+1} (G8) is Cys all six strategies (2.1-2.6) can be used to inhibit Step 2 (Transesterification reaction), which results in a total of 2⁸1=63 strategies for inhibiting Step 2. The application of the strategies 2.1-2.6 defined above to the unprocessed intein are described below.

To inhibit Step 2 using Strategy 2.1, the amino acid at position I_{C+1} (G8) needs to be mutated to an amino acid that cannot function as a nucleophile in Step 2 (Transesterification reaction). The inteins listed in InBase, contain Ser, Cys, and Thr at position I_{C+1} (G8). Mutation of I_{C+1} (G8) to any other amino acid should inhibit Step 2 (Transesterification reaction). However, certain inteins are only able to use a specific nucleophilic amino acid at position I_{C+1} (G8) (Shingledecker, K. et al. (2000) Archives Biochem. Biophys. 375:138-144). Therefore, for these inteins, Step 2 (Transesterification reaction) can be inhibited by substituting the wild-type amino acid for another nucleophilic amino acid at position I_{C+1} (G8). (Shingledecker, K. et al. (2000) Arch. Biochem. Biophys. 375:138-144). For example, in Psp Pol-I intein, Step 2 (Transesterification reaction) is inhibited, when Ser I_{C+1} (G8) is mutated to Cys or Thr, (Xu, M. & Perler, F. B. (1996) EMBO J. 15:5146-5153). Similarly, mutation of Cys at position I_{C+1} (G8) to Ser inhibits Step 2 (Transesterification reaction) in the Sce VMA1 intein (Hirata, R. & Anraku, Y. (1992) Biochem. Biophys. Res. Commun. 188:40-47).

To inhibit Step 2 using Strategy 2.2, the amino acid at position B7 needs to be mutated to an amino acid that cannot hydrogen bond to the carbonyl oxygen at position I_{N+1} (A1). The following amino acids at position B7 occur more than once in the Inteins listed in InBase: Thr, Ser, Asn, Asp, Cys, and Glu. Mutation of the amino acids at position B7 to any other amino acid except Thr, Ser, Asn, Asp, Cys, and Glu, should inhibit Step 2 (Transesterification reaction). However, certain inteins are able to only use a specific amino acid at position B7. Therefore, for these inteins, Step 2 (Transesterification reaction) can be inhibited by substituting the wild type amino acid for any other amino acid at position B7.

To inhibit Step 2 using Strategy 2.3, the amino acid at position B10 needs to be mutated to an amino acid that cannot hydrogen bond with the amido nitrogen at position I_{N+1} (A1). The most common amino acids at position B10 in inteins listed in InBase that are believed to undergo splicing are His and Thr. The amino acids Asp and Lys also occur at position B10 although at a much lower frequency than His and Thr. These amino acids are capable of hydrogen bonding with the amido nitrogen at position I_{N+1} (A1) and mutation of amino acids at position B10 to any other amino acid except His, Thr, Asp, and Lys should inhibit Step 2 (Transesterification reaction). However, certain inteins are able to only use a specific amino acid at position B10. Therefore, for these inteins, Step 2 (Transesterification reaction) can be inhibited by substituting the wild-type amino acid for another amino acid at position B10.

To inhibit Step 2 using Strategy 2.4, a charged amino acid is introduced near the splice site. The inteins listed in InBase that are believed to undergo splicing contain primarily Leu, Val, Phe, and Ile at position A2 (I_{N+2}). The amino acids His, Gln, Met, Gly, Cys, Ser, Thr, and Tyr occur in less than ten inteins at position A2 (I_{N+2}). At position G5 (I_{C-3}), the most frequently occurring amino acids are Val, Thr, Leu, Ala, and Ser. The amino acids Cys, Ile, Asn, and His occur in four or less inteins. The most common amino acids at positions A2 (I_{N+2}) and G5 (I_{C-3}) are hydrophobic. Introduction of Lys, Arg, Glu, or Asp near the splice site should inhibit Step 2 (Transesterification reaction). However, certain inteins are able to only use a specific amino acid at positions A2 (I_{N+2}) and G5 (I_{C-3}). Therefore, for these inteins, Step 2 (Transesterification reaction) can be inhibited by substituting the wild-type amino acid for another amino acid at positions A2 (I_{N+2}) and G5 (I_{C-3}).

To inhibit Step 2 using Strategy 2.5, the amino acid at position F4 needs to be mutated to an amino acid that cannot hydrogen bond with the carbonyl oxygen of the I_{N-1}. The inteins listed in InBase that are believed to undergo splicing primarily contain Asp, Cys, Thr, Trp, Ser, and Asn at the F4 position. Amino acids Arg, Ala, Glu, Phe, Gly, Ile, Leu, Gln, Val, and Tyr occur in five or less inteins. The amino acids Asp, Cys, Thr, Trp, Ser, and Asn can all form hydrogen bonds with the carbonyl oxygen of the I_{N-1}. Mutation of the amino acid at position F4 to an amino acid that does not form hydrogen bonds should inhibit Step 2 (Transesterification reaction). However, certain inteins are able to only use a specific amino acid at position F4. Therefore, for these inteins Step 2 (Transesterification reaction) can be inhibited by substituting the wild-type amino acid for another amino acid at position F4.

To inhibit Step 2 using Strategy 2.6, the I_{C+1} (G8) nucleophile needs to be Cys. When the I_{C+1} (G8) nucleophile is Cys, addition of Zinc to the growth media will inhibit Step 2 (Transesterification reaction).

Generation of Unprocessed Intein By Inhibiting Step 1 (N—X Acyl Shift)

Unprocessed intein can also be generated using a single strategy or a combination of strategies that inhibit only Step 1 (N—X acyl shift). There are three strategies (1.1-1.3) to inhibit Step 1 (N—X acyl shift), which results in a total of 2³−1=7 strategies for inhibiting Step 1 (N—X acyl shift). Application of strategies 1.1-1.3 for generating unprocessed intein are described below.

To inhibit the Step 1 using Strategy 1.1 the amino acid at position Al (I_{N+1}) needs to be mutated to an amino acid that cannot function as a nucleophile in Step 1 (N—X acyl shift). The inteins listed in InBase that are believed to undergo splicing primarily contain Cys,

Ser, and to a lesser extent Ala. Inteins with Ala at this position undergo the alternative intein mechanism described above. In standard inteins, mutation of the amino acid at position Al to any other amino acid should inhibit Step 1 (N—X acyl shift). However, certain inteins are able to only use a specific nucleophilic amino acid at position Al (I_{N+1}). Therefore, for these inteins, Step 1 (N—X acyl shift) can be inhibited by substituting the wild-type amino acid for another nucleophilic amino acids at position Al (I_{N+1}). For example, the Psp Pol-I intein splices poorly when Ser I_{N+1} (A1) is mutated to Cys, and splicing is blocked completely when Ser is mutated to Thr (Xu, M. & Perler, F. B. (1996) EMBO J. 15:5146-5153). Similarly, the Sce VMA1 intein does not tolerate a mutation of Cys at position I_{N+1} (A1) to Ser (Hirata, R. & Anraku, Y. (1992) Biochem. Biophys. Res. Commun. 188:40-47, Cooper, A. A. et al. (1993) EMBO J. 12:2575-2583).

To inhibit the Step 1 using Strategy 1.2, the amino acid at position F3 needs to be mutated to an amino acid the disrupts N—X catalytic pocket. The inteins listed in InBase that are believed to undergo splicing primarily contain Tyr and Phe at the F3 position. Amino acids Glu, Ile, Arg, Val, Gln, Asp, Lys, Thr, His, Leu, Trp, Ser, Cys, Gly, Asn, and Pro occur less often at this position. Mutation of amino acids at position F3 to any amino acid other than Tyr or Phe will inhibit Step 1 (N—X acyl shift). However, certain inteins are able to only use a specific amino acid at position F3. Therefore, for these inteins, Step 1 (N—X acyl shift), can be inhibited by substituting the wild-type amino acid for another amino acid at position F3.

To inhibit the Step 1 using Strategy 1.3, amino acids within hydrogen bonding distance of the side chain of the I_{N+1} (A1) nucleophile need to be mutated. The amino acids found here do not correspond to amino acids in the conserved intein blocks. Thr and Arg are within hydrogen bonding distance of the side chain of the I_{N+1} (A1) nucleophile in the Ssp DnaE and Ssp DnaB inteins. Mutation of Thr or Arg to an amino acid that cannot hydrogen bond to the side chain of the I_{N+1} (A1) nucleophile will inhibit Step 1 (N—X acyl shift), or Step 2 (Transesterification).

Generation of Unprocessed Intein By Inhibiting Step 1 (N—X Acyl Shift) and Step 3 (Asn Cyclization)

A stable unprocessed intein can be generated using a single or a combination of strategies that inhibit Step 1 (N—X acyl shift) with a single or combination of strategies that inhibit Step 3 (Asn Cyclization). There are three strategies (1.1-1.3) to inhibit Step 1 (N—X acyl shift) and five strategies (3.1-3.5) to inhibit Step 3 (Asn cyclization), which results in a total of (2³−1)×(2⁵−1)=217 different strategies for inhibiting Steps 1 (N—X acyl shift) and 3 (Asn Cyclization). Application of the strategies 1.1-1.3 for generating unprocessed intein are described above. Application of strategies 3.1-3.5 for generating unprocessed intein are described below.

To inhibit the Step 3 using Strategy 3.1, the amino acid at position I_{C-1} (G7) needs to be mutated to an amino acid that cannot undergo cyclization. The inteins listed in InBase that are believed to undergo splicing contain Asn and Gin at position 1_{C}_{31 1} (G7). Mutation of amino acids at position I_{C-1} (G7) to an amino acid that cannot undergo side chain cyclization will inhibit Step 3 (Asn cyclization). However, certain inteins are able to only use a specific amino acid at position I_{C-1} (G7). Therefore, for these inteins, Step 3 (Asn cyclization) can be inhibited by substituting the wild-type amino acid for another amino acid at position I_{C-1} (G7).

To inhibit the Step 3 using Strategy 3.2, amino acids G6 (I_{C-2}) and/or B11, which assist in Asn cyclization by hydrogen bonding with the Asn carbonyl oxygen at position I_{C-1} (G7) should be mutated to an amino acid that cannot hydrogen bond with this amino acid. The inteins listed in InBase that are believed to undergo splicing contain His at the G6 (I_{C-2}) position and to a lesser extent Gly, Ser, Ala, and Cys. Mutation to any amino acid except for His should inhibit Step 3 (Asn cyclization). In the absence of the His at G6 (I_{C-2}), B11 can assist in Asn cyclization by hydrogen bonding with the Asn carbonyl oxygen at position I_{C-1} (G7). B11 is predominately Lys or Arg when G6 is not His. Mutation to any amino acid that does not have a positive charge (Lys, Arg, or His) at either position should inhibit Step 3 (Asn cyclization) However, certain inteins are able to only use a specific amino acid at position G6 (I_{C-2}) or B11. Therefore, for these inteins, Step 3 (Asn cyclization) can be inhibited by substituting the wild-type amino acid for another amino acid at position G6 (I_{C}₋₂) or B11.

To inhibit the Step 3 using Strategy 3.3, the amino acid at position F13 needs to be mutated to an amino acid that cannot act as a proton acceptor from Asn at position I_{C-1} (G7) through a coordinated water molecule. The inteins listed in InBase that are believed to undergo splicing contain primarily His, and to a lesser extent, Glu, Gln, Asn, Pro, Ser, Lys, Ala, Gly, Asp, Arg, Ile, Leu, Tyr, Trp, Val, and Thr at position F13. If the wild-type residue is His mutation to another amino acid should inhibit Step 3 (Asn cyclization). However, certain inteins are able to only use a specific amino acid at position F13. Therefore, for these inteins, Step 3 (Asn cyclization) can be inhibited by substituting the wild-type amino acid for another amino acid at position F13.

To inhibit the Step 3 using Strategy 3.4, the amino acid at position F14 needs to be mutated to an amino acid that inhibits Asn cyclization. The inteins listed in InBase that are believed to undergo splicing contain primarily Asn at position F14. The amino acids, Leu, Ser, Thr, Gln, Ala, Arg, Met, Phe, Val, Glu, Tyr, His, Lys, Cys, Asp, and Ile occur less frequently. Mutation to any other amino acid would disrupt the splice site inhibiting Step 3 (Asn cyclization). However, certain inteins are able to only use a specific amino acid at position F14. Therefore, for these inteins, Step 3 (Asn cyclization) can be inhibited by substituting the wild type amino acid for another amino acid at position F14.

To inhibit the Step 3 using Strategy 3.5, the amino acid at position F15 needs to be mutated to an amino acid that inhibits Asn cyclization. The inteins listed in InBase that are believed to undergo splicing contain primarily Phe and Tyr at position F15. The amino acids Val, Gly, Asn, Ser, Thr, His, Ile, Trp, Ala, and Glu also occur at position F15. The F15 position forms hydrophobic contacts with amino acids surrounding the splice site and orients the amino acid at position F13. Mutation of the amino acid at position F15 from Phe or Tyr to any amino acid except Phe or Tyr will inhibit Step 3 (Asn cyclization). However, certain inteins are able to only use a specific amino acid at position F15. Therefore, for these inteins, Step 3 (Asn cyclization) can be inhibited by substituting the wild type amino acid for another amino acid at position F15.

Generation of Unprocessed Intein By Inhibiting Step 1 (N—X Acyl Shift) and Step 2 (Transesterification)

Three strategies (1.1-1.3) can be used to inhibit Step 1 (N—X acyl shift), If I_{C+1} (G8) is not Cys then strategy 2.6 is not applicable and there are only five strategies (2.1-2.5) that can be used to inhibit Step 2 (Transesterification), which results in a total of (2³−1)×(2⁶−1)=217 strategies for inhibiting Steps 1 (N—X acyl shift) and 2 (Transesterification). If I_{C+1} (G8) is Cys then there are six strategies (2.1-2.6) that can be used to inhibit Step 2 (Transesterification), which results in a total of (2³−1)×(2⁶−1)=441 strategies for inhibiting Steps 1 (N—X acyl shift) and 2 (Transesterification). Application of strategies 1.1-1.3 and 2.1-2.6 for generating unprocessed intein are described above.

Generation of Unprocessed Intein By Inhibiting Step 2 (Transesterification) and Step 3 (Asn Cyclization)

Five strategies (3.1-3.5) can be used to inhibit Step 3 (Asn cyclization). If I_{C+1} (G8) is not Cys then strategy 2.6 is not applicable and there are only five strategies (2.1-2.5) to inhibit Step 2 resulting in (2⁵−1)'(2⁵−1) =961 strategies for inhibiting Steps 1 (N—X acyl shift) and 3 (Asn Cyclization). If I_{C+1} (G8) is Cys then there are six strategies (2.1-2.6) that can be used to inhibit Step 2 (Transesterification), which results in a total of (2⁶−1)×(2⁵−1)=1953 strategies for inhibiting Steps 1 (N—X acyl shift) and 3 (Asn Cyclization). The application of the strategies 2.1-2.6 and 3.1-3.5 for the unprocessed intein are described above.

Generation of Unprocessed Intein by Inhibiting Step 1 (N—X Acyl Shift), Step 2 (Transesterification), and Step 3 (Asn Cyclization)

Three strategies (1.1-1.3) can be used to inhibit Step 1 (N—X acyl shift), and five strategies (3.1-3.5) can be used to inhibit Step 3 (Asn cyclization). If I_{C+1} (G8) is not Cys then strategy 2.6 is not applicable, which leaves five strategies (2.1-2.5) to inhibit Step 2 resulting in (2³−1)×(2⁵−1)×(2⁵−1)=6727 strategies for inhibiting Steps 1 (N—X acyl shift), 2 (Transesterification), and 3 (Asn Cyclization). If I_{C+1} (G8) is Cys then six strategies (2.1-2.6) can be used to inhibit Step 2 resulting in (2³−1)×(2⁸−1)×(2⁵−1)=13671 strategies for inhibiting Steps 1 (N—X acyl shift), 2 (Transesterification), and 3 (Asn Cyclization). The application of the strategies 1.1-1.3, 2.1-2.6, and 3.1-3.5 for the unprocessed intein are described above.

Description of Dicysteine Intein

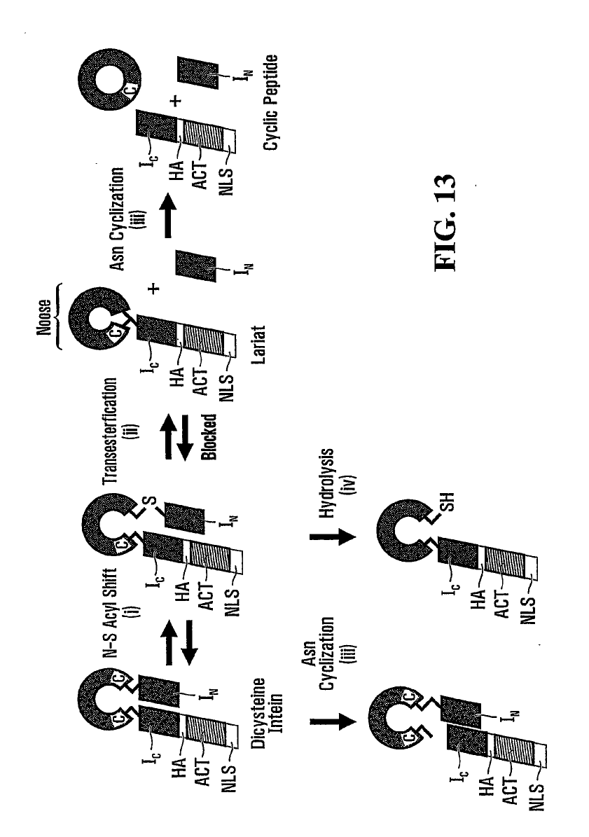

The dicysteine intein does not undergo any steps in the intein-mediated splicing reaction (FIG. 13). Cys amino acids at positions I_{C+1} (G8) and I_{N+1} (A1) are retained and other mutations are required to inhibit intein processing. After dicysteine inteins are selected that interact with a given target, a peptide containing the random peptide, ScFv, or genomic fragment flanked by the cysteine residues can be synthesized. These peptides, ScFvs, and genomic fragments can then be constrained by disulfide bonds or cysteine cross-linking reagents.

The Cys amino acids at positions I_{C+1} (G8) and I_{N+1} (A1) are required for the dicysteine intein. Strategy 2.2 (Zinc inhibition) is a good strategy for generating the dicysteine intein as it does not require mutation at I_{C+1} (G8), and inhibition is reversible. Alternatively using an intein that is not tolerant to substitutions at I_{C+1} (G8) and I_{N+1} (A1) can be used to generate the dicysteine intein. For example the Psp Pol intein has Ser at positions I_{C+1} and I_{N+1} and mutation to Cys inhibits protein splicing (Xu, M. & Perler, F. B. (1996) EMBO J. 15:5146-5153).

Generation of Dicysteine Intein By Inhibiting Step 1 (N—X Acyl Shift)

Dicysteine intein can be generated using a single strategy or a combination of strategies that inhibit only Step 1 (N—X acyl shift). There are three strategies (1.1-1.3) to inhibit Step 1 (N—X acyl shift), which gives rise to 2³−1=7 strategies for inhibiting Step 1 (N—X acyl shift). If the intein has a native Cys at position I_{N+1} (A1) then strategy 1.1 cannot be used. Therefore there are 2²−1=3 strategies for inhibiting Step 1 (N—X acyl shift). The application of the strategies 1.1-1.3 for the dicysteine intein are described below.

In Strategy 1.1 the amino acid at position I_{N+1} (A1) needs to be mutated to Cys or if it is already a Cys no changes need to be made. Certain inteins are able to only use a specific nucleophilic amino acid at position I_{N+1} (A1). Therefore, for these inteins, Step 1 (N—X acyl shift) can be inhibited by substituting the wild-type amino acid for another nucleophilic amino acid at position I_{N+1} (A1). For example, Psp Pol-I intein splices poorly when Ser I_{N+1} (A1) is mutated to Cys (Xu, M. & Perler, F. B. (1996) EMBO J. 15:5146-5153).

To inhibit the Step 1 (N—X acyl shift) using Strategy 1.2, the amino acid at position F3 needs to be mutated to an amino acid the disrupts N—X acyl shift catalytic pocket. Inteins listed in InBase that are believed to undergo splicing primarily contain Tyr and Phe at the F3 position. Amino acids Glu, Ile, Arg, Val, Gin, Asp, Lys, Thr, His, Leu, Trp, Ser, Cys, Gly, Asn, and Pro occur less often at this position. Mutation of amino acids at position F3 to any amino acid other than Tyr or Phe should inhibit Step 1 (N—X acyl shift). However, certain inteins are able to only use a specific amino acid at position F3. Therefore, for these inteins, Step 1 (N—X acyl shift) can be inhibited by substituting the wild-type amino acid for another amino acid at position F3.

To inhibit the Step 1 using Strategy 1.3, amino acids within hydrogen bonding distance of the side chain of the I_{N+1} (A1) nucleophile need to be mutated. The amino acids in this region do not correspond to amino acids in the conserved intein blocks. Thr51 and Arg50 are within hydrogen bonding distance of the side chain of the I_{N+1} (A1) nucleophile in the Ssp DnaE and Ssp DnaB inteins. Mutation of Thr or Arg to an amino acid that cannot hydrogen bond to the side chain of the I_{N+1} (A1) nucleophile should inhibit Step 1 (N—X acyl shift) or Step 2 (Transesterification reaction).

Generation of Dicysteine Intein By Inhibiting Step 1 (N—X Acyl Shift) and Step 3 (Asn Cyclization)

A stable dicysteine intein can be generated by using a single or a combination of strategies that inhibit Step 1 (N—X acyl shift) with a single or combination of strategies that inhibit Step 3 (Asn Cyclization). There are three strategies to inhibit Step 1 (N—X acyl shift) (1.1-1.3) and five strategies to inhibit Step 3 (Asn Cyclization), which results in a total of (2³−1)×(2⁵−1)=217 strategies to inhibit Steps 1 (N—X acyl shift) and 3 (Asn Cyclization). If the intein has a Cys at position I_{N+1} (A1), then Strategy 1.1 is not relevant. For this case, there are a total of (2²−1)×(2⁵−1)=93 strategies to inhibit Steps 1 (N—X acyl shift) and 3 (Asn Cyclization). The application of the strategies 1.1-1.3 for generating the dicysteine intein are described above. The application of strategies 3.1-3.5 for generating the dicysteine intein are the same as for the unprocessed intein.

Generation of the Dicysteine Intein by Inhibiting Step 1 (N—X Acyl Shift) and Step 2 (Transesterification)

A stable dicysteine intein can be generated by using a single or a combination of strategies that inhibit Step 1 (N—X acyl shift) with a single or combination of strategies that inhibit Step 2 (Transesterification). There are three strategies to inhibit Step 1 (N—X acyl shift) (1.1-1.3) and six strategies to inhibit Step 2 (Transesterification), which results in a total of (2³−1) (2⁶−1)=441 strategies to inhibit Steps 1 (N—X acyl shift) and 2 (Transesterification). If the wild-type amino acid for the intein is a Cys at position I_{N+1} (A1), then Strategy 1.1 is not relevant. For this case, there are a total of (2²−1)×(2⁶−1)=189 strategies to inhibit Steps 1 (N—X acyl shift) and 2 (Transesterification). If the intein has a Cys at position I_{C+1} (G8), then Strategy 2.1 is not relevant. For this case, there are a total of (2³−1)×(2⁵−1)=217 strategies to inhibit Steps 1 (N—X acyl shift) and 2 (Transesterification). If the wild-type amino acids for the intein at positions I_{C+1} (G8) and I_{N+1} (A1) are both Cys then Strategies 1.1 and 2.1 are not relevant. In this case there are a total of (2²−1)×(2⁵−1)=93 strategies to inhibit Steps 1 (N—X acyl shift) and 2 (Transesterification). See unprocessed intein for description of mutations that inhibit Step 2 (Transesterification reaction), except for Strategy 2.1, which is discussed below.

in Strategy 2.1, the amino acid at position I_{C+1} (G8) needs to be mutated to Cys. Certain inteins are able to only use a specific nucleophilic amino acid at position I_{C+1} (G8) and mutation to Cys will inhibit Step 2 (Transesterification). Therefore, for these inteins, Step 2 (Transesterification) can be inhibited by substituting the wild-type amino acid for another nucleophilic amino acid at position I_{C+1} (G8). For example, in Psp Pol-I intein, Step 2 (Transesterification reaction) is inhibited, when Ser I_{C+1} (G8) is mutated to Cys (Xu, M. & Perler, F. B. (1996) EMBO J. 15:5146-5153).

Generation of the Dicysteine Intein by Inhibiting Step 2 (Transesterification) and Step 3 (Asn Cyclization)

A stable dicysteine intein can be generated by using a single or a combination of strategies that inhibit Step 2 (Transesterification) with a single or combination of strategies that inhibit Step 3 (Asn cyclization). There are six strategies (2.1-2.6) to inhibit Step 2 (Transesterification) and five strategies (3.1-3.5) to inhibit Step 3 (Transesterification), which results in a total of (2⁶−1)×(2⁵−1)=1953 strategies to inhibit Steps 2 (Transesterification) and 3 (Asn cyclization). If the wild-type amino acid at position I_{C+1} (G8) is Cys then strategy 2.1 is not relevant, which leaves five strategies (2.2-2.6) to inhibit Step 2 (Transesterification). In this case there are (2⁵−1)×(2⁵−1)=961 strategies to inhibit Steps 2 (Transesterification) and 3 (Asn cyclization). See unprocessed intein for description of mutations that inhibit Steps 2 (Transesterification reaction) and 3 (Asn cyclization), except for Strategy 2.1 described above).

Generation of the Dicysteine Intein by Inhibiting Step 1 (N—X Acyl Shift), Step 2 (Transesterification), and Step 3 (Asn Cyclization)

A stable dicysteine intein can be generated by using a single or a combination of strategies that inhibit Step 1 (N—X acyl shift), with a single or combination of strategies that inhibit Step 2 (Transesterification reaction), and with a single or combination of strategies that inhibit Step 3 (Asn cyclization). There are three (1.1-1.3) strategies to inhibit Step 1 (N—X acyl shift), six strategies (2.1-2.6) to inhibit Step 2 (Transesterification reaction) and five strategies (3.1-3.5) to inhibit Step 3 (Asn cyclization), which results in a total of (2³−1)×(2⁶−1)×(2⁵−1)=13671 strategies to inhibit Steps 1 (N—X acyl shift), 2 (Transesterification), and 3 (Asn cyclization). If the wild-type amino acid for the intein is a

Cys at position I_{N+1} (A1), then Strategy 1.1 is not relevant. For this case, there are a total of (2²−1)×(2⁶−1)×(2⁵−1)=5859 strategies to inhibit Steps 1 (N—X acyl shift), 2 (Transesterification), and 3 (Asn cyclization). If the intein has a Cys at position I_{C+1} (G8), then Strategy 2.1 is not relevant. For this case, there are a total of (2³−1)×(2⁵−1)×(2⁵−1)=6727 strategies to inhibit Steps 1 (N—X acyl shift), 2 (Transesterification), and 3 (Asn cyclization). If the wild-type amino acids for the intein at positions I_{C+1} (G8) and I_{N+1} (A1) are both Cys then Strategies 1.1 and 2.1 are not relevant. In this case there are a total of (2²−1)×(2⁵−1)×(2⁵−1)=2883 strategies to inhibit Steps 1 (N—X acyl shift), 2 (Transesterification), and 3 (Asn cyclization). See unprocessed intein for description of mutations that inhibit Step 1 (N—X acyl shift), Step 2 (Transesterification reaction), and Step 3 (Asn cyclization), except for Strategies 1.1 and 2.1, which are discussed above).

Description of the Lariat Intein

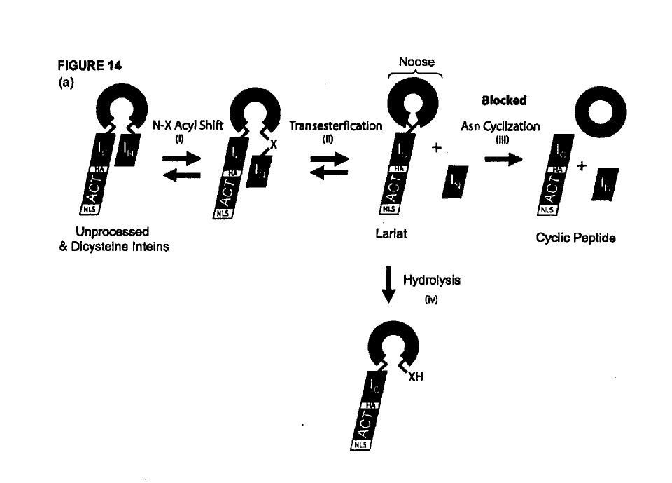

The lariat intein is generated by allowing the first two steps of intein reaction (FIG. 14) to proceed and by blocking the third step, (Asn cyclization). Any residues surrounding I_{C+1} (G8) that stabilizes the ester bond from hydrolysis should also be incorporated. Mutations that enhance the first two steps are also beneficial. Strategy 4 may also be beneficial for generating robust lariat libraries, where an increased number of library members form lariats.

Generation of the Lariat Intein by Inhibiting Step 3 (Asn Cyclization)

The lariat intein can be generated using strategies that inhibit Step 3 (Asn cyclization) or combinations of strategies that inhibit Step 3 (Asn cyclization). There are six strategies that inhibit Step 3 (Asn cyclization), which gives rise to 2⁵−1=31 strategies for inhibiting Step 3 (Asn cyclization). Strategy 2.6 might also be applicable since it is not definitively confirmed that Zinc blocks Step 2 (Transesterification) (Mills, K. V. & Paulus, H. (2001) J. Biol. Chem. 276:10832-10838).

in Strategy 3.1 the amino acid at position I_{C-1} (G7) needs to be mutated to an amino acid that cannot undergo cyclization. The inteins listed in InBase that are believed to undergo splicing contain Asn and Gln at position I_{C-1} (G7). Mutation of amino acids at position I_{C-1} (G7) to any amino acid other than Asn, Gln and Asp will inhibit Step 3 (Asn cyclization). Specific mutations of I_{C-1} (G7) can also lead to stabilization of the branched intermediate (Lariat). Mutation of the I_{C-1} G7 amino acid to Lys (Kawasaki M. et al. (1997) J. Biol. Chem. 272:15668-15674), Gln, or Asp, (Xu, M. & Perler, F. B. (1996) EMBO J. 15:5146-5153) may facilitate the stabilization of the branched intermediate (lariat). Accumulation of the branched intermediate is also observed when this amino acid is not mutated, but amino acids at position G6 (I_{C-2}) are mutated to Leu, Asn, or Gin (Xu, M. & Perler, F. B. (1996) EMBO J. 15:5146-5153).

In Strategy 3.2 amino acids G6 (I_{C-2}) and/or B11, which assist in Asn cyclization by hydrogen bonding with the Asn carbonyl oxygen at position I_{C-1} (G7) should be mutated to an amino acid that cannot hydrogen bond with this amino acid. The inteins listed in InBase that are believed to undergo splicing contain His at the G6 (I_{C-2}) position and to a lesser extent Gly, Ser, Ala, and Cys. Mutation to any amino acid except for His should inhibit Step 3 (Asn cyclization). In the absence of the His at G6 (I_{C-2}), it has been found that B11 can hydrogen bonding with the Asn carbonyl oxygen at position I_{C-1} (G7). Position B11 is predominately Lys or Arg when G6 is not His. Mutation to any amino acid that does not have a positive charge (Lys, Arg, His) at either position should inhibit Step 3 (Asn cyclization). Mutation of His at position I_{C-2}(G6) in the Psp pol-I intein to Leu, Asn, and Gin results in an accumulation of the branched intermediate only when I_{C-1} (G7) is not mutated to Ala (Xu, M. & Perler, F. B. (1996) EMBO J. 15:5146-5153). Currently, there are no mutagenic studies on the role of Arg at position B11 in accumulating branched intermediates. However, certain inteins are able to only use a specific amino acid at positions G6 (I_{C-2}) or B11. Therefore, for these inteins, Step 3 (Asn cyclization) can be inhibited by substituting the wild-type amino acid for another amino acid at positions G6 (I_{C-2}) or B11.

For Strategies 3.3 to 3.5 see unprocessed intein for description of mutations that inhibit Step 3 (Asn cyclization).

Application of the Lariat Intein Technology to Isolate Lariats that Bind to and Inhibit the Bacterial Repressor Protein Lexa

The present invention describes the construction and application of the “lariat”, unprocessed intein, and dicysteine intein in the yeast two-hybrid assay. The lariat is a new peptide construct that has no C-terminus and represents a novel class of cyclic peptides. Lariat peptides are generated by modifying the in vivo intein-mediated protein ligation reaction. The C-terminus of the lariat peptide is looped back and linked to a specific serine n the interior of the peptide via a cyclic lactone bond (FIG. 3). The lariat has a free N-terminus that allows the attachment of useful biological domains such as an activation domain, which is necessary for yeast-two hybrid assays.

As discussed above this method is used to generate cyclic peptide, lariat, unprocessed intein, and dicysteine intein affinity agents against a given target. The feasibility of this approach is demonstrated by generating inhibitors of the bacterial repressor protein LexA. LexA represents a putative antimicrobial target, which when inhibited should potentiate that activity of cytotoxic antibiotics. When LexA is bound by activated RecA it undergoes autoproteolysis and no longer represses genes in its regulon (Lin, L. L. & Little, J. W. (1988) Bacteriol. 170:2163-2173). LexA mutants that block autoproteolysis (Walker, G. C. (1984) Microbiol. Rev. 48:60-93) make bacteria more sensitive to stress induced by compounds such as the DNA damaging reagent mitomycin C (MMC) (Lin, L. L. & Little, J. W. (1988) Bacteriol. 170:2163-2173) and they decrease antibiotic resistance (Cirz, R. T. et al. (2005) PLoS Biol. 3:e176, Miller, C. et al. (2004) Science 305:1629-1631). LexA inhibitors that block autoproteolysis would increase the sensitivity of bacteria to cytotoxic reagents and since LexA is not present in humans it would have no effect on host DNA damage repair systems.

Construction and Screening of Combinatorial Lariat Peptide Libraries

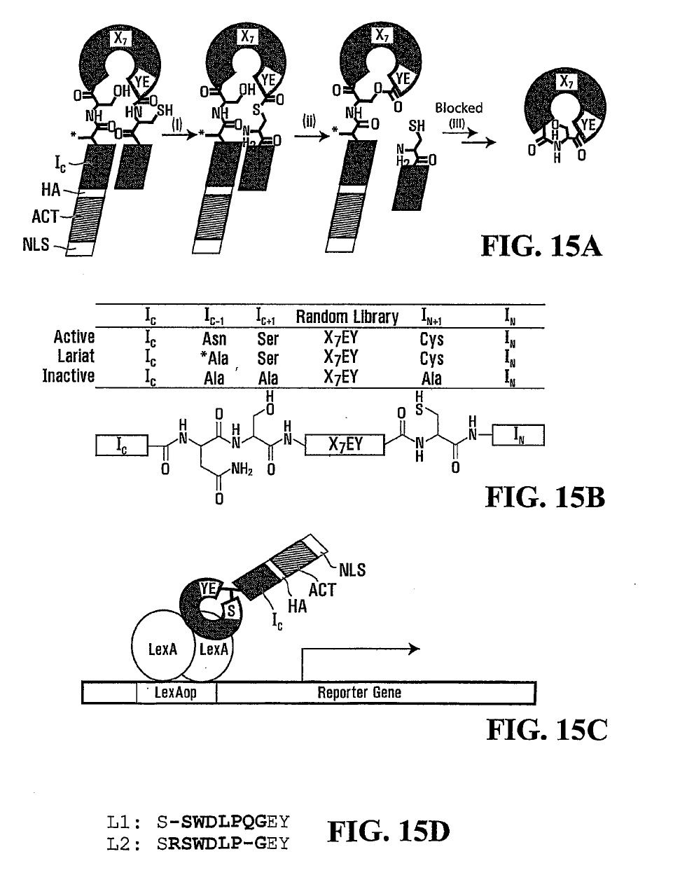

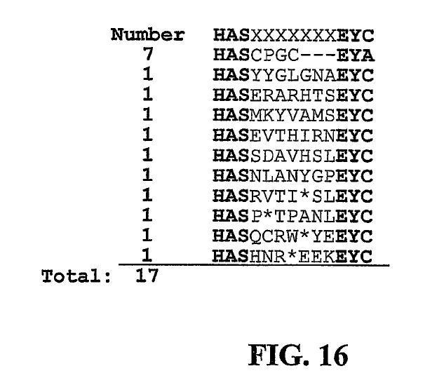

Lariats were generated that are compatible with the yeast two-hybrid system by engineering the intein producing cyclic peptide system (Scott, C. P. et al. (1999) Proc. Natl. Acad. Sci. USA 96:13638-13643) to halt the cyclic peptide reaction at an intermediate step, which produces a lariat that contains a transcription activation domain covalently attached through an amide bond to a lactone-cyclized peptide. To prevent the lariat intermediate from undergoing asparagine cyclization, which produces a cyclic peptide, asparagine at position I_{C-1} (G7) was substituted with alanine (FIG. 15a, b). A combinatorial library of lariats was created, where the “noose” region contains the amino acid sequence SXXXXXXXEY, where X represents amino acids encoded by the NNK codon. Glutamate and tyrosine amino acids in the noose region are included to facilitate cyclization (Scott, C. P. et al. (2001) Chem Biol. 8:801-815, Naumann, T. A. et al. (2005) Biotechnol. Bioeng. 92:820-830). A library of approximately seven million lariat peptides was constructed in the MATa yeast strain EY93 (FIG. 16) and mated the library to a MATa strain EY111 containing the LexA target plasmid (pEG202) (Gyuris, J. et al. (1993) Cell 75:791-803) and yeast two-hybrid reporter genes. Using the yeast two-hybrid interaction trap in FIG. 15c, 14 clones were isolated, encoding two unique lariats that interacted with LexA (FIG. 15d). The L2 lariat was used for further analysis as it contains more charged amino acids, which may enhance its solubility.

Characterization of Anti-LexA Lariats

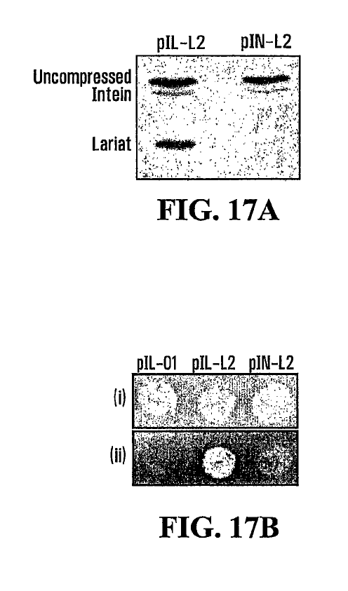

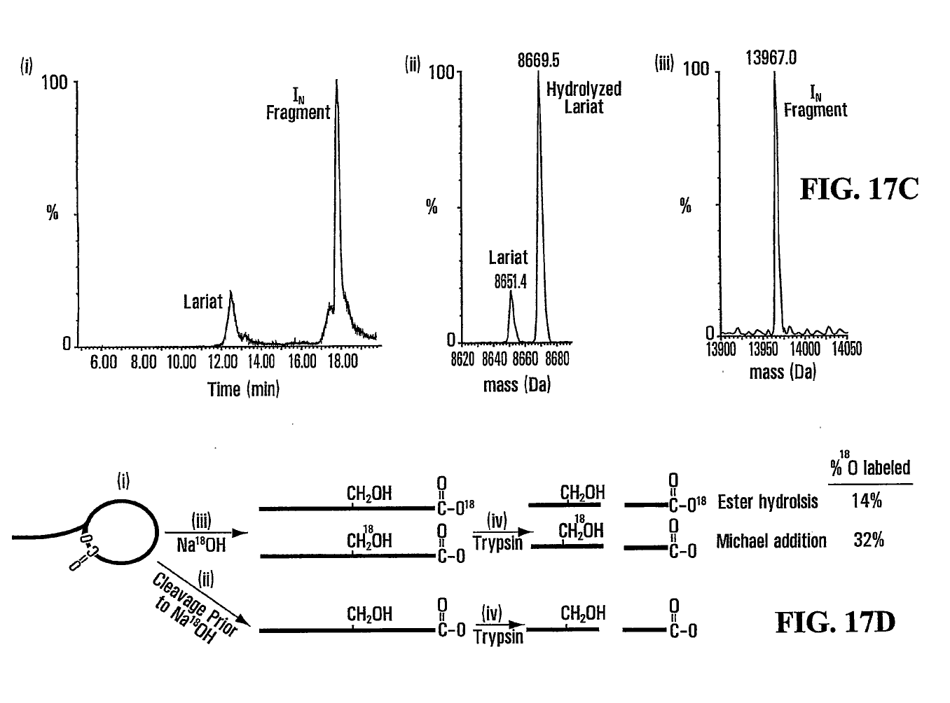

To confirm the importance of the lariat structure for the L2 lariat-LexA interaction, the noose region from the L2 lariat was cloned into an inactive lariat intein plasmid (pIN-L2), which does not undergo any steps in the intein-mediated cyclization reaction. We confirmed expression of the L2 lariat and the L2 inactive lariat intein in EY93 and monitored the intein-mediated cyclization reaction, using Western analysis with an antibody against the N-terminal intein haemagglutinin (HA) tag (FIG. 17a). pIL-L2 produces unprocessed (˜23 kDa) and lariat (˜9 kDa) products, whereas inactive intein plasmid pIN-L2 produces only unprocessed product. The lariat structure is important for the L2 lariat-LexA interaction, as activation of the yeast two-hybrid reporter genes with the inactive L2 intein (pIN-L2), expressing the unprocessed lariat, is barely detectable relative to the L2 lariat (pIL-L2), which expresses both the unprocessed product and the lariat (FIG. 17b).

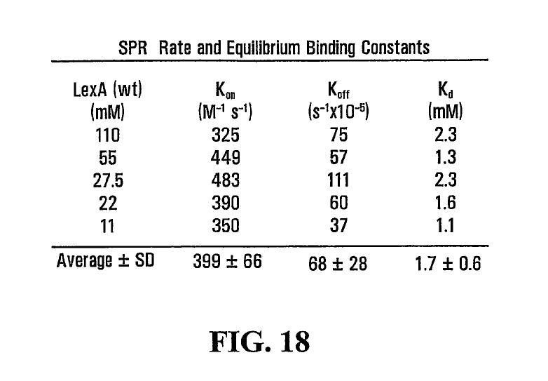

We used surface plasmon resonance analysis to determine whether a direct interaction occurs between a synthetic linear peptide corresponding to the L2 lariat noose and LexA. The linear L2 peptide interacted with LexA with a K_{d} of 1.7±0.6 μM (FIG. 18).

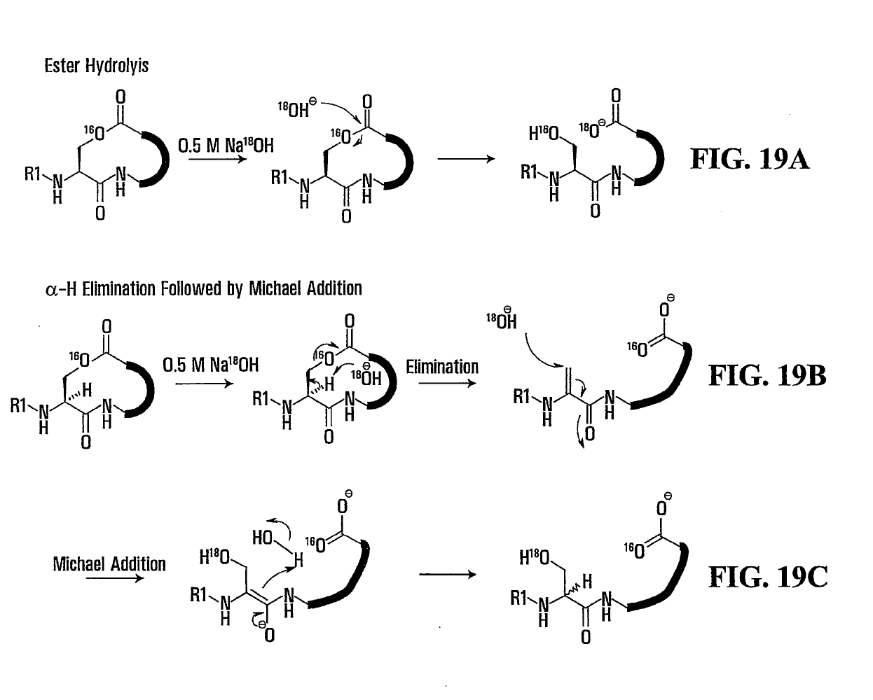

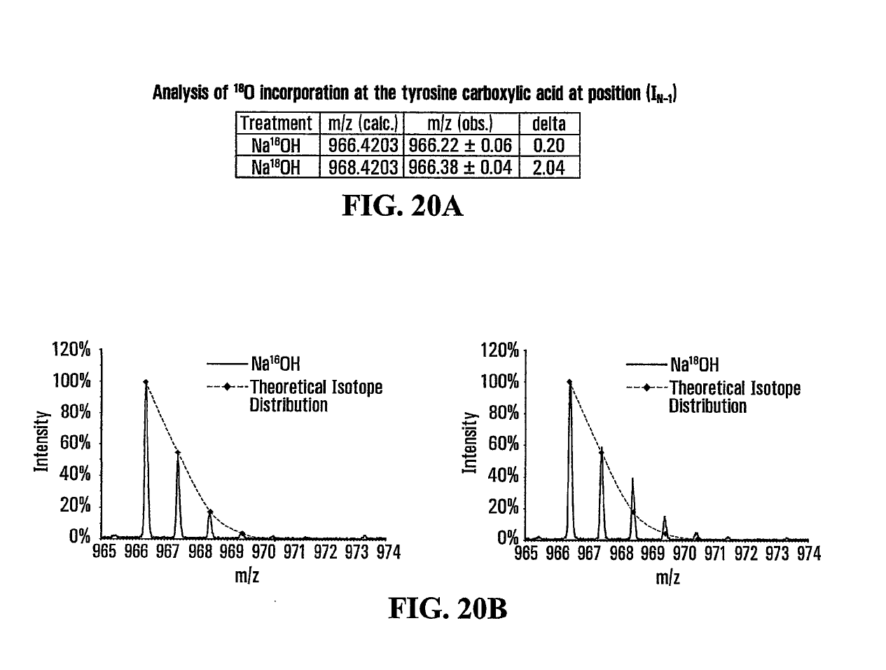

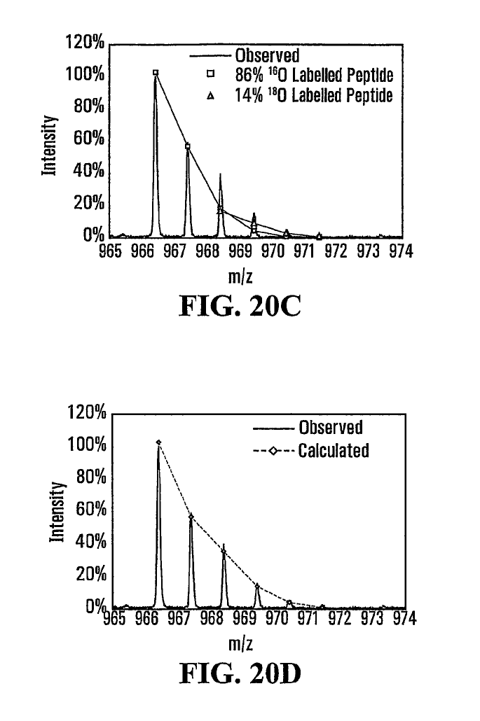

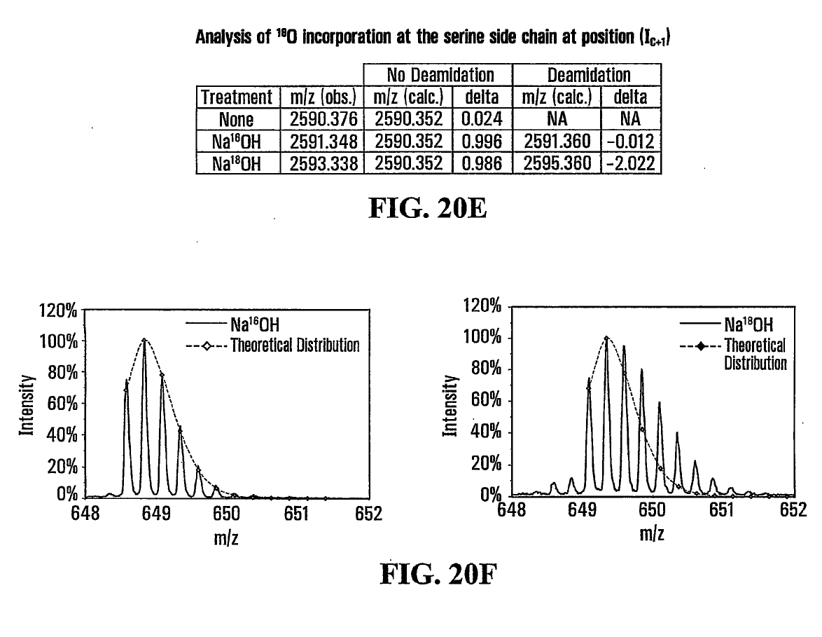

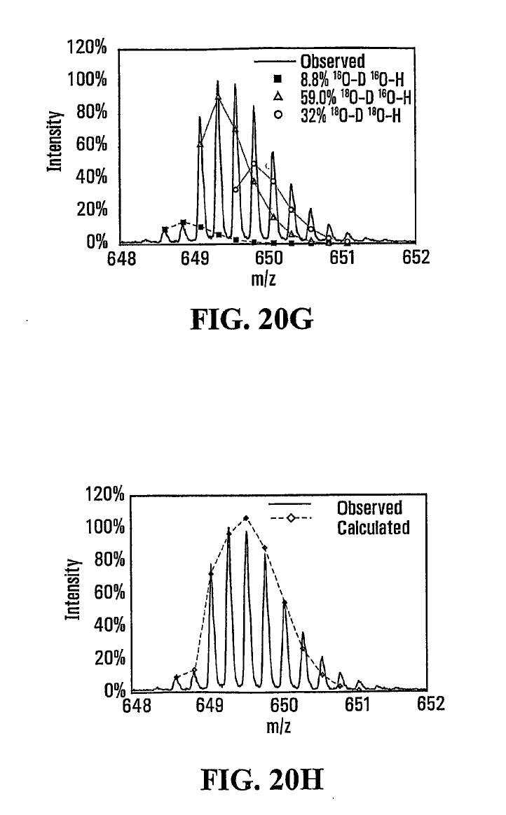

We used mass spectrometry (MS) to measure the molecular weight of the L2 lariat expressed from a His-tag bacterial expression vector (pETIL-L2) in BL21 CodonPlus (BL21-CP) E. coil. We observed two products, 15% corresponds to the L2 lariat (8651 Da) and 85% corresponds to a hydrolyzed lariat product that is 18 Da heavier (8669 Da) (FIG. 17c). Lariats are difficult to observe by MS (Scott, C. P. et al. (1999) Proc. Natl. Acad. Sci. USA 96:13638-13643, Scott, C. P. et al. (2001) Chem Biol. 8:801-815), presumably due to hydrolysis of the lactone bond caused by high temperatures and acidic conditions used in the MS analysis. To determine the amount of lariat present prior to MS analysis, we forced the cleavage of the lariat lactone using Na¹⁸OH (Hagelin, G. (2005) Rap. Commun. Mass Spectrom. 19:3633-3642) and then digested the lariat with trypsin and analyzed the molecular weight of the fragments using LC-ESI-TOF MS (FIG. 17d). Lariat lactones cleaved by Na¹⁸OH are 2 Da heavier than lactones cleaved prior to Na¹⁸OH treatment. We observed incorporation of ¹⁸O into two trypsin fragments that results either from the hydrolysis of the ester bond or from an α-H elimination that generates dehydroalanine, followed by a Michael addition (FIG. 19). The fraction of ¹⁸O incorporated in these fragments indicates that 46% of the lariat is cyclized prior to MS analysis (FIG. 20). This data, combined with the fact that many lactone-cyclized peptides exist in nature (Guenewald, J. & Marahiel, M. A. (2006) Microbiol. Mol. Biol. Rev. 70:121-146), supports the existence of the lariat structure in vivo.

Biological Activity of Anti-LexA Lariat and Cyclic Peptide

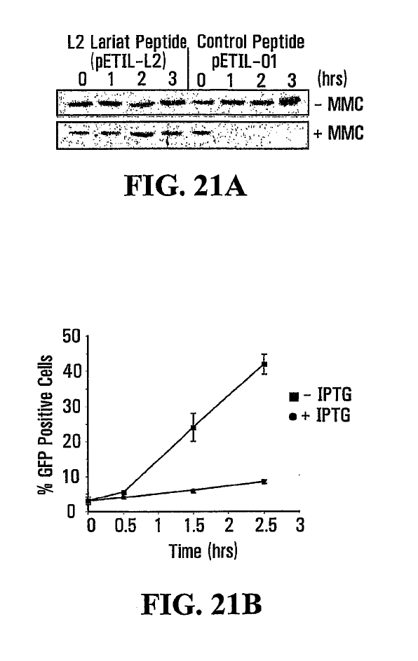

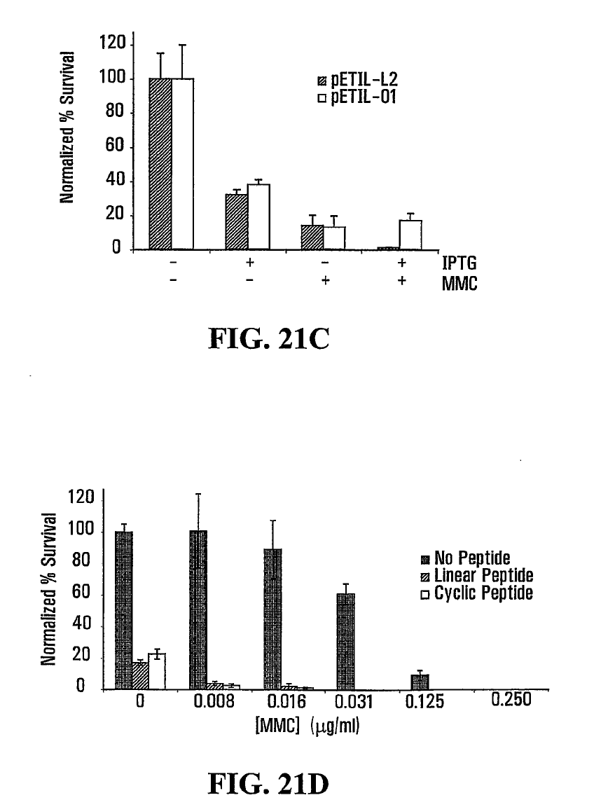

We monitored the ability of L2 lariat to block MMC-induced LexA cleavage. MMC is a potent inducer of bacterial SOS response that activates the RecA coprotease activity and induces cleavage of LexA (Lin, L. L. & Little, J. W. (1988) Bacteriol. 170:2163-2173). We transformed pETIL-L2 into BL21-CP and used Western analysis to monitor degradation of LexA after exposure to MMC in the presence and absence of the L2 lariat (Yasuda, T. et al. (1998) EMBO J. 17:3207-3216) (FIG. 21a). LexA cleavage is not observed after three hours in cells that express L2 lariat, whereas in cells that express a lariat intein with a CPGC amino acid noose (pETIL-01) LexA is completely cleaved after one hour.

We confirmed that expression of L2 lariat blocks MMC-induced expression of SOS response genes. We engineered the E. coil strain SMR6039 that expresses GFP under the control of a SOS-regulated sulA promoter (Hastings, P. J. et al. (2004) PLoS Biol. 2:e399) to express T7 RNA polymerase (SMR6039-DE3), which allows expression of the L2 lariat from the T7 promoter. MMC treatment of SMR6039-DE3 transfected with pETIL-L2 in the absence of inducer (IPTG) results in a time-dependent increase in the percentage of GFP expressing cells (FIG. 21b). Expression of L2 lariat peptide by the addition of IPTG decreases the percentage of GFP expressing cells (FIG. 21b).

We tested the ability of L2 lariat to inhibit bacterial growth in the presence and absence of MMC using the survival assay described by Lin and Little (Un, L. L. & Little, J. W. (1988) Bacteriol. 170:2163-2173). We expressed L2 lariat (pETIL-L2) or a lariat with a CPGC noose (pETIL-01) in BL21-CP cells, exposed the bacteria to MMC in 0.85% NaCl for one hour, and assayed their survival (FIG. 21c). Expression of either plasmid reduces the viability to ˜35% of the uninduced controls. MMC alone (0.1 μg/mL) reduced the viability to ˜14% of the untreated control. Expression of L2 lariat enhanced the activity of MMC and reduced the viability to <1% of the control, whereas expression of the lariat with CPGC noose did not enhance the activity of MMC.

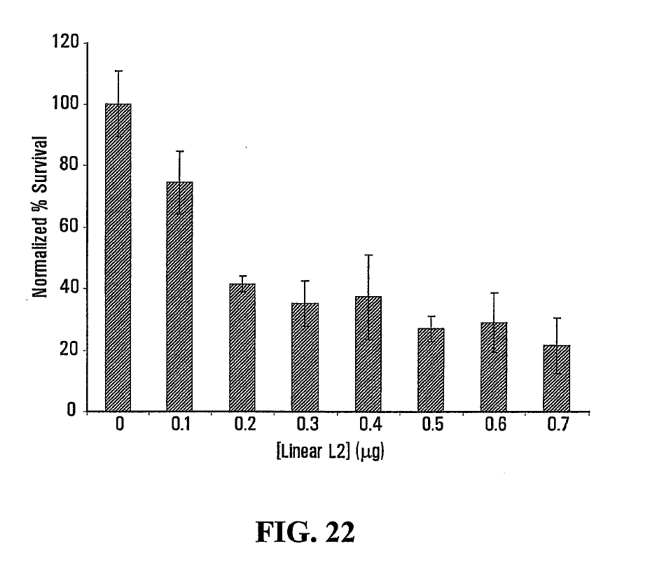

We synthesized cyclic and linear peptides that correspond to the L2 lariat noose and tested their ability to inhibit bacterial growth and potentiate the activity of MMC. First, we examined the ability of L2 peptides alone to inhibit bacterial growth using the survival assay in 0.85% NaCl⁹. Treatment of BL21-CP with cyclic or linear L2 peptides reduced the survival of BL21-CP to -20% of the untreated control (FIG. 21c). No further decrease in survival is observed when linear L2 peptide is increased from 0.2 to 0.7 μg/mL (FIG. 22). Next, we examined the ability of L2 peptides to potentiate the effects of MMC. We monitored cell survival at a constant L2 peptide concentration and varied the MMC concentration. Cyclic and linear L2 peptides decreased the minimal inhibitory concentration of MMC by approximately 10-fold (FIG. 21d).

Accordingly, the invention provides methods to genetically select lariats against a given target protein using intein-mediated peptide cyclization and the yeast two-hybrid interaction trap. This system allows lariats and cyclic peptides based on the noose sequence of the lariats to be rapidly generated against protein targets that are compatible with the yeast two-hybrid system. The lariat technology provides a rapid high throughput system for isolating cyclic peptide inhibitors that can be used for the reverse analysis of protein function or as drugs or pseudo-drugs for validating therapeutic targets.

We used this system to generate lariat inhibitors of LexA and validate LexA as a therapeutic target for potentiating the antimicrobial effects of reagents that activate the SOS response pathway. The lariats can be converted to cyclic or linear peptides that also potentiate the effects of MMC.

Methods

Reagents.

Linear peptides are from the University of Calgary Rapid Multiple Peptide Synthesis Service (Calgary, AB). Cyclic peptides are from Anygen Co. Ltd. (Korea). Oligonucleotides are from IDT DNA (Coralville, Iowa) and are listed in Supplementary Table 1 online.

Strains and Plasmids.

E. coli strains: BL21(DE3) is from Novagen (Madison, Wis.) and BL21-CodonPlus®(DE3)-RIL (BL21-CP) is from Stratagene (La Jolla, Calif.). SMR6039 is a gift from Susan Rosenberg (Hastings, P. J. et al. (2004) PLoS Biol. 2:e399).

S. cerevisiae strains: EY93 (MATa ura2 his3 trp1 leu2 ade2::URA3) is a derived from EGY42 (Cohen, B. A. et al. (1998) Proc. Natl. Acad. Sci. USA 95:14272-14277). EY111 (MATα his3 trp1 ura3::LexA8op-lacZ ade2::URA3-LexA8op-ADE2 leu2::LexA6op-LEU2) is derived from EGY48 (Golemis, E. A. & Brent, R. (1992) Mol. Cell. Biol. 12:3006-3014).

pIN01: The lariat intein design is based on the amino acid sequence of the Synechocystis spp. strain PCC6803 (Ssp) DnaE intein gene. We assembled the inactive intein gene by mixing 0.1 μg of each of the eight oligonucleotides [A-H) with 2.5 units of pfu polymerase (Fermentas, Burlington, ON), 200 μM dNTPs, 20 mM Tris-Cl, 10 mM (NH₄)₂SO₄, 10 mM KCl, 0.1% (v/v) Triton X-100, 0.1 mg/mL bovine serum albumin (BSA), and 2 mM MgSO₄. We incubated the assembly reaction for 5 minutes at 95° C., then performed 25 cycles of 30 seconds at 95° C., 30 seconds at 50° C. and 1.5 minutes 72° C., followed by a final incubation for 10 minutes at 72° C. We amplified the inactive intein gene using ⅕ (10 μL) of the assembly reaction in a 50 μl PCR reaction containing 1 μM PCR primers I and J using the reaction conditions and amplification cycles described above. We used lithium acetate transformation (Schiestl, R. H. & Gietz, R. D. (1989) Curr. Genet. 16:339-346) with 500 ng of EcoRI/XhoI (Fermentas) digested pJG4-5 (Gyuris, J. et al. (1993) Cell 75:791-803) and 400 ng of PCR amplified inactive intein to clone the inactive intein into pJG4-5 by in vivo homologous recombination in EY93 (Ma, H. et al. (1987) Gene 58:201-216).

Lariat Library (pIL-XX): We replaced the CPGC linker peptide in pIN01 with a combinatorial seven amino acid peptide using oligonucleotide K. We PCR amplified oligonucleotide K using primers L and M. We used the reaction conditions described above with seven amplification cycles consisting of a denaturing step at 95° C. for 30 seconds, an annealing step at 55° C. for 30 seconds, and an extension step at 72° C. for 15 seconds. We digested pIN01 with RsrII (New England Biolabs, Ipswich, Mass.) and dephosphorylated the digested plasmid with 10 units of shrimp alkaline phosphatase (Fermentas). We cloned the library into pIN01 using in vivo homologous recombination (Ma, H. et al. (1987) Gene 58:201-216) in EY93. We performed 100 lithium acetate transformations (Schiestl, R. H. & Gietz, R. D. (1989) Curr. Genet. 16:339-346) with each transformation containing 400 ng of amplified oligonucleotide K and 1 μg of RsrII-digested pIN01. In total, we obtained 20 million yeast colonies.

pIL-L2: pIL-L2 is a library member from the pILXX library. The noose sequence is (RSWDLPGEY).

pIN-L2: We constructed pIN-L2 by mutating cysteine at I_{N+}1 to alanine, which produces an inactive intein. Two overlapping PCR fragments were used to introduce the point mutation. We used primers I and N to amplify the N-terminus region and primers O and J to amplify the C-terminal region. We mixed the two PCR products together and amplified the full-length intein with primers I and J. We cloned the PCR fragment into EcoRI/XhoI-digested pIN01 using in vivo homologous recombination in EY93 (Ma, H. et al. (1987) Gene 58:201-216).

pETIL-L2: We constructed pETIL-L2 by PCR amplifying the entire pIL-L2 intein gene including the stop codon with primers P and Q. We digested the PCR fragment with EcoRI and XhoI (Fermentas) and cloned it into pET28b (Novagen).

pETIL-01: We constructed pETIL-01 by PCR amplifying the entire pIN-01 intein gene including the stop codon using primers P and Q. We digested the PCR fragment with EcoRI and XhoI (Fermentas) and cloned it into pET28b (Novagen).

Characterization of the Lariat Library

We isolated pIL-XX plasmids from an overnight culture of EY93 containing pIL-XX in Trp⁻ glucose media using the “Smash and Grab” yeast mini-prep (Geyer, C. R. & Brent, R. (2000) Methods Enzymol. 328:178-208). We electroporated 3 μL of the yeast mini-prep into MC1061 E. coil cells (Invitrogen, Burlington, ON) and selected for transformants on Luria broth (LB) with 100 μg/mL ampicillin (LB-AMP). We isolated the plasmids from 17 of the transformants using a Qiagen bacteria mini-prep kit (Qiagen, Mississauga, ON). We sequenced the seven amino acid combinatorial peptide insert using primer R and ABI big Dye terminator chemistry (Applied Biosciences Inc, Foster City, Calif.).

Screening of Combinatorial Lariat Intein Library.

We screened the lariat library for interactions with LexA using yeast two-hybrid interaction mating (Kolonin, M. G. et al. (2000) Methods Enzymol. 328:26-46). We transformed the LexA bait plasmid (pEG202) (Gyuris, J. et al. (1993) Cell 75:791-803) into EY111 and mated EY111::pEG202 to EY93::pIL-XX. We cultured EY111::pEG202 in 500 mL of His⁻ glucose media to an OD₆₀₀ of 0.6-0.9. We pelleted EY111::pEG202 cells by centrifugation and resuspended the pellet in an equal volume of yeast peptone dextrose (YPD) media. We mixed EY93::pIL-XX cells with EY111::pEG202 cells at a ratio of 1:20. We mated the yeast cells on YPD plates at 30° C. for 24 hours. We pooled the mated yeast cells and screened 20 million diploid yeast cells to detect lariats that interact with LexA using the LEU2, ADE2 and LacZ reporter genes. We cultured diploid yeast cells on His⁻Trp⁻Leu⁻Ade⁻ galactose/sucrose plates containing X-Gal for approximately seven days. We selected positive colonies and reconfirmed positive interactions by isolating pILXX from the positive colonies and repeating the yeast two-hybrid assay as described above.

Characterization of Intein Processing and Lariat Product.

We monitored expression of the lariat in EY93 using Western analysis with an anti-HA antibody. We incubated EY93 containing pIL-L2 or pIN-L2 overnight at 30° C. in Trp⁻ galactose/raffinose media. We collected the cells by centrifugation, resuspended the cells in 300 μL bead buffer (20 mM Tris-Cl pH 7.9, 10 mM MgCl₂ 1 mM EDTA, 5% Glycerol, 1 mM DTT, 0.3 M (NH₄)SO₄, 1 mM PMSF) and 500 μL of acid-washed glass beads (Sigma, Oakville, ON), and lysed the cells in a FastPrep FP120 (Q-Biogene, Irvine, Calif.). We cleared the cell lysate by centrifugation at 4° C. We normalized the samples using their OD₆₀₀ and analyzed 20 μL of supernatant using standard Western analysis procedures (Ausubel, F. M. et al. (1997) Current protocols in Molecular Biology) with an anti-HA tag antibody (Santa Cruz Biotechnology, Santa Cruz, Calif.) (1:200 dilution).

We used LC-ESI-TOF MS to confirm the molecular weight of the His-tag lariat purified from E. coli. To confirm the presence of the lariat lactone prior to MS analysis, we treated His-tag lariat with 0.5 M Na¹⁸OH, purified the products using reverse-phase HPLC, digested them with trypsin, and analyzed the molecular weight of the trypsin fragments using LC-ESI-TOF MS.

Analysis of LexA Autoproteolysis.

We monitored the effect of the L2 lariat on MMC-induced LexA autoproteolysis using an anti-LexA antibody. We grew overnight cultures of BL21-CP::pETIL-01 or BL21-CP::pETIL-L2 in 10 mL of LB with 30 μg/mL kanamycin (LB-KAN). We diluted cultures to an OD₆₀₀ of 0.1 in LB-KAN with 1 mM IPTG and cultured the cells at 30° C. to an OD₆₀₀˜0.4-0.6. We treated cells with 100 μg/mL chloramphenicol, incubated them for 10 minutes, and split the culture in two. We treated one culture with 0.1 μg/mL MMC and left the second culture untreated. We removed 4 mL samples from each culture at indicated time points, washed the cells with H₂O, and stored them at −80° C. until all time points were taken. We resuspended the cells in 250 μL of PBS Triton X-100 (0.05%) and ˜300 μL acid-washed glass beads (Sigma) and homogenized 4× in a FastPrep FP120 (Q-biogene). We centrifuged the cell lysates at 4° C. and analyzed the cleared supernatants using standard Western analysis procedures (Ausubel, F. M. et al. (1997) Current protocols in Molecular Biology) with an anti-LexA antibody (Invitrogen) (1:5000 dilution).

Analysis of MMC-Induced Expression of SOS Response Genes.

We used the SMR6039 E. coli strain, which expresses GFP under the control of a SOS-regulated sulA promoter (Hastings, P. J. et al. (2004) PLoS Biol. 2:e399) to monitor induction of the SOS response pathway. We used the λDE3 Lysogenization Kit (Novagen) to modify SMR6039 to express T7 RNA polymerase, which allows expression of the L.2 lariat from the T7 promoter in pETIL-L2 plasmid. We cultured SMR6039(DE3) containing pETIL-L2 overnight in LB-KAN, diluted the cultures to an OD₆₀₀=0.1 in LB-KAN with 1 mM IPTG, and cultured the cells to an OD₆₀₀˜0.4-0.6. We treated the cells with 0.1 μg/mL MMC, removed samples at specified time points, and diluted them in 2 mL 0.85% NaCl for a final concentration of ˜0.5×10⁶ cfu/mL. We measured the GFP fluorescence of the samples using flow cytometery (Epics X L, Coultier, Mississauga, ON). We scored cells as positive for SOS induction in they expressed more than one fluorescence unit of GFP.

Bacterial Viability Assays.

We performed cell viability assays as described by Lin and Little (Lin, L. L. & Little, J. W. (1988) Bacteriol. 170:2163-2173). For assays where L2 lariat is expressed from pET28b plasmid, we cultured BL21(DE3)-CP containing pETIL-L2 or pETIL-01 to an OD₆₀₀ of 0.4 in LB-KAN at 37° C. We split the samples in two and induced one sample with 1 mM IPTG for 1 hour and left the other the other sample uninduced. We diluted the samples 100-fold in 5 mL 0.85% NaCl with or without 0.1 μg/mL of MMC. We removed 10 μL and diluted it 1000-fold in ice cold LB (1 mL) for a zero time point control. We incubated the remaining sample at 37° C. for 1 hour and then removed 10 μL and diluted 1000-fold into 1 mL ice-cold LB. We plated a 60 μl aliquot from the 0 and 1 hour samples on LB plates and incubated the plates at 37° C. overnight. For assays using synthetic linear and cyclic L2 peptide, we performed the survival assay as described above except instead of inducing the cells prior to MMC treatment, we added 0.7 μg/mL of peptide. Normalized percent cell survival is calculated by dividing the number of colony forming units (cfu) after one hour by the number of cfu at the zero hour time point. The uninduced control or the no peptide control is normalized to 100%.

Surface Plasmon Resonance Analysis of L2 peptide-LexA Interaction

We synthesized linear L2 peptide with a TAT importer sequence (Vive's, E. et al. (1997) Biol. Chem. 272:16010-16017) at the N-terminal: NH₂-GRKKRRQRRRPPQ-SRSWDLPGEY. We attached the peptide to a carboxymethylated dextran matrix sensor chip (CM5, Biacore, Piscataway, N.J.) using the manufacture's protocol. We purified LexA proteins as described previously (Little, J. W. et al. (1994) Methods Enzymol. 244:266-284). We determined the binding kinetics of the L2 peptide-LexA interaction by injecting LexA in 50 mM Phosphate Buffer Saline, 100 mM NaCl at 20 μL/minute for 2 minutes and measuring the dissociation constant for 1.5 minute on a BiacoreX (Biacore). We determined the binding kinetics for LexA concentrations ranging from 11 μμM-110 μM. Binding curves for each dilution were fitted for kₒₙ and k_{off} rates using the BiaEvaluation software (Biacore).

Purification and Characterization of His-Tag Purified Lariat

We purified the His-tag lariats using a Ni-NTA Spin Kit (Qiagen). Briefly, we transformed BL21-CP E. coli (Invitrogen) with pIL-L2. We expressed the L2 lariat by inducing a 0.4 OD₆₀₀ culture of BL21-CP::pIL-L2 with 1 mM IPTG for three hours. We washed the cells, suspended them in phosphate buffered saline, 0.05% Triton X-100, 1 mg/mL lysozyme and used sonication to lyse them. We centrifuged the lysate at 10,000×g for 20 minutes at 4° C. and passed the clarified supernatant through a Ni—NA column (Qiagen). We washed the column 3 times with 50 mM NaH₂PO₄ and 300 mM NaCl and eluted the L2 lariat using 50 mM NaH₂PO₄ pH 7.0, 250 mM NaCl, and 100 mM EDTA. We separated and desalted the His-tag purified lariats using a C4 reverse phase column (Symmetry300® C4 3.5 μm 2.1×50 mm Column) (Waters, Milford, Mass.) with a gradient of 5% Buffer A/95% Buffer to 25% Buffer A/75% Buffer B over 20 minutes (Buffer A: H₂O and 0.1% Formic acid (v/v), Buffer B: Acetonitrile and 0.08% Formic acid (v/v)). We determined the molecular weights of the eluted proteins using ESI(+)-TOF MS (MicroMass LCT, Waters). We resolved the multi-charged lariat spectrums using maximum entropy software (MaxEnt3, Waters).

To determine the amount of lactone-cyclized lariat in the sample prior to MS analysis, we forced the cleavage of the lactone bound using Na¹⁸OH. We prepared 0.5 M Na¹⁸OH by dissolving sodium (Sigma) in 98% H₂¹⁸O (Stable Isotopes, Summit, N.J.). We lyophilized His-tag purified L2 lariat and treated 500 μg of the L2 lariat with either 0.5 M Na¹⁸OH or 0.5 M Na¹⁸OH for 16 hours at room temperature (4). We acidified the reaction with 0.5 N HCl to give a final pH between 2.0 to 7.0. We purified the lariat sample by HPLC under the same conditions described previously using a C4 reverse phase column (Symmetry300™ C4 3.5 μm 2.1×50 mm Column (Waters)). We lyophilized the purified L2 lariat and resuspended it in 6 M Urea and 100 mM Tris-HCl pH 8.0 and heated the sample at 80° C. for 10 minutes to denature the protein. We cooled the sample to room temperature, diluted it 10-fold in 100 mM Tris-HCl pH 8.0, added 0.68 μg of modified sequencing grade trypsin (Roche, Laval, QC), and incubated the sample overnight (18 hours) at 37° C. We separated the tryptic digests from the Na¹⁶OH or the Na¹⁸OH treated samples on a BioSuite™ C18 PA-A 3 μm 2.1×250 mm Column (Waters) using a gradient of 5% Buffer A/95% Buffer B to 50% Buffer A/50% Buffer B over 20 minutes (Buffer A: H₂O and 0.1% Formic acid (v/v), Buffer B: Acetonitrile and 0.08% Formic acid (v/v)). We analyzed eluted peptides were analyzed using a ESI-TOF(+) MS (MicroMass LCT, Waters).

We processed the raw spectra using MATCHING software (Fernandez-de-Cossio, J. et al. (2004) Rap. Commun. Mass Spectrom. 18:2465-2472), which detects proteins that have small differences in molecular weight by comparing the observed isotopic pattern to the predicted isotopic pattern. MATCHING software was used to calculate the percentage of ¹⁶O and ¹⁸O incorporation in the two tryptic peptide fragments involved in the lactone bond, SWDLPGEY [966.42 m/z] [amino acids 73-80] and IFDIGLPQDHNFLLANGAIAHASR [2590.352 m/z][amino acids 49-72]. For the 73-80 amino acid fragment, MATCHING software was used to determine the percentage of ¹⁶O and ¹⁸O incorporation assuming one oxygen incorporation. For the 49-72 amino acid fragment, MATCHING was used to determine the percentage of ¹⁶O and ¹⁸O incorporation assuming two oxygen incorporations.

We used the constraints generated by MATCHING software to calculate the maximum intensity of the mixture of peptides and plotted the calculated peak intensities against the observed peak intensities. First, we calculated the intensity of each peak using equation 1 (EQ1). Each peak was assigned an index j=1 . . . N, where N is the number of peaks. The maximum intensity of each peptide is defined by (x), where i=1 . . . P and P is the number of peptides. Each peptide has an associated isotopic distribution based on its molecular formula and the percentage of heavy isotopes found in nature. This distribution was determined using MS-ISOTOPE software (Clauser, K. R. et al. (1999) Anal. Chem.

71:2871-2882). The intensity of each peak is the sum of the maximum intensity of all peptides found in that peak multiplied by a scalar factor (Iⱼ), which is the percentage of the maximum intensity of the peptide at that peak location predicted by MS-ISOTOPE software.

The total error (R²) between the observed intensity (lobsⱼ) and the calculated intensity (Icalcⱼ) for all peaks (from j=1 . . . N) is defined by equation 2 (EQ2). We substituted the system of equations, one equation for each peak (j), from EQ1 into EQ2. EQ2 was then simplified to a single variable by applying the constraints given by MATCHING software. For amino acids 73-80 (SWDPLGEY), MATCHING software determined the ratio to be 14% ¹⁶O to 86% ¹⁸0. For amino acids 49-72 (IFDIGLPQDHNFLLANGAIAHASR), MATCHING software determined the ratio to be 8.8% for two ¹⁸O incorporations, 59.0% for one ¹⁶O incorporation and one ¹⁸O incorporation and 32.2% for two ¹⁸O incorporations. We took the derivative of EQ2 and solved for the minimum total error with respect to the single variable. This value gives the maximum intensity of one of the peptide species, which is used to calculate the values of the other peptide intensities.

Equations:

EQ1: Calculated peak intensity

Σ(xᵢIⱼ)=Icj

i is the number of different peptides in the model, j is the peak index, xᵢ is the maximum intensity of a peptide, Iⱼ is a scale factor determined by MS-ISOTOPE for the fraction of xᵢ expected at that peak location, and Icᵢ is the calculated peak intensity from the model at that peak location.

EQ2: Total Error

Σ(Iₒⱼ−I_{Cj})=R²

j is the peak index, lobsᵢ is the intensity observed, and Icalcⱼ is the calculated intensity.

Creation and Screening of a Mixed Lariat Intein Library

Amino acids in the extein at the intein-extein junction can effect splicing. The Ssp DnaE intein has been shown to be promiscuous in regards to the amino acids that are found adjacent to the splice site. Mutation of wild-type inteins or using mixed inteins can alter this dependency. Iwai et al., (Iwai, H. et al. (2006) FEBS Lett. 580:1853-1858) showed that a split-intein with the Ssp DnaE I_{C}domain and the Nostoc punctiforme (Npu) DnaE I_{N} domain could more efficiently ligate linear extein with a wider variety of amino acids at the I_{C}-extein junction, (I_{C}+2) than the wt Ssp DnaE intein.

Dassa et al. (Dassa, B. et al. (2007) Biochemistry 46:322-330) tried all combinations of N-terminal and C-terminal domains from, Nostoc sp. PCC7120 (Nsp), Oscillatoria limnetica (Oli), and Thermosynechococcus Vulcanus (Tvu). All of these combinations underwent some splicing demonstrating that split-inteins from different species can associate and that various combinations spliced more efficiently than the wild-type inteins. This association is thought to be in part partially due to charge-charge interactions between the negatively charged amino acids found 14 amino acids immediately preceding block B and the positively charged amino acids found 12 amino acids immediately preceding block F, including the F1 amino acid.

Based on these findings, we constructed mixed intein libraries with the I_{N} domain from Npu DnaE and the I_{C}domain from Ssp DnaE. As described previously, we generated lariats that are compatible with the yeast two-hybrid system by engineering the intein producing cyclic peptide system (Scott, C. P. et al. (1999) Proc. Natl. Acad. Sci. USA 96:13638-13643) to halt the cyclic peptide reaction at an intermediate step, which produces a lariat that contains a transcription activation domain covalently attached through an amide bond to a lactone-cyclized peptide. To prevent the lariat intermediate from undergoing Asn cyclization, which produces a cyclic peptide, we mutated Asn at position I_{C-1} (G7) to Ala. The plasmid backbone was modified to include a different selectable marker (Kan instead of Amp) as well as containing the I_{N} domain of the Npu DnaE intein and the I_{C}domain of the Ssp DnaE intein.

To verify this construct still processed, the L2 peptide (SRSWDLPGEY) isolated against LexA using the intein containing both domains from Ssp DnaE (I_{C}-Ssp, I_{N}-Ssp) was transferred to the (I_{C}-Ssp, I_{N}-Npu) intein the L2 peptide still interacted with LexA in a yeast-two-hybrid assay and underwent processing.

We created three combinatorial libraries of lariats. One library where the “noose” region contains the amino acid sequence SX₍₁₀₎, where X represents amino acids encoded by the NNK codon (R10). Two libraries where the “noose” region contains the amino acid sequence SX₍₅₎, where X represents amino acids encoded either by the NNK codon (R5), or the BNT codon (B=G, T, or C)(F5). Libraries of lariat peptides were constructed in the MATa yeast strain EY93. Library construction was confirmed by sequencing. The R5 and F5 library diversity was greater than the theoretical diversities of 3×10⁷ and 2.5×10⁵ respectively at the nucleotide level. The R10 library diversity was 6.5×10⁶.

Ten library copies of each library was mated to the PR domain of Riz1 as well as various domains of Jak2 including full-length Jak2 V617F, Tyrosine Kinase domain (JH1), Pseudokinase domain (JH2 V617F), and the Tyrosine Kinase domain fused to pseudokinase domain (JH1-JH2 V617F). The strongest hits from each screen were isolated, the plasmids obtained and their interactions were rechecked in the yeast two-hybrid assay. The PR domain screen resulted in three different lariat sequences that specifically bound the PR domain. Two sequences were from the R10 library and one sequences was from the R5 library. From the lariats against Jak2 that have been analyzed, there is one lariat against the JH1 domain, three different lariats against the JH2 V617F domain, and four different lariats against the full-length Jak2 V617F. All of these lariats are from the R10 library.

Methods

Construction of the (I_{C}-Ssp, I_{N}-Npu) Intein Plasmids

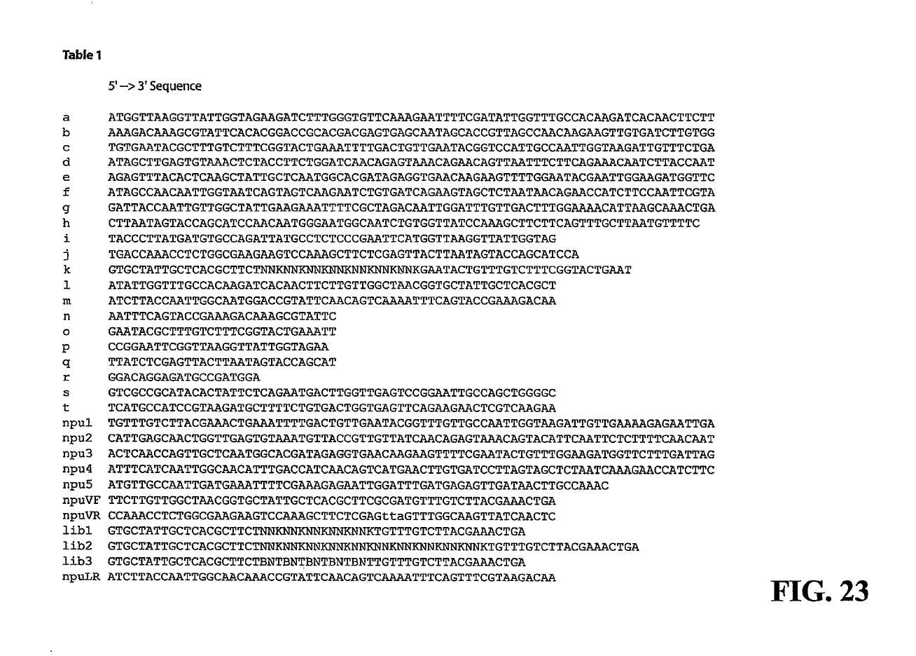

pIN01 was digested with RsrII and XhoI in NEBuffer 4 [50 mM Tris-Acetate pH 7.9, 50 mM Potassium Acetate, 10 mM Magnesium Acetate, 1 mM Dithiothreitol] for 3 hrs at 37° C. to remove the Ssp DnaE I_{N} domain. Synthetic Npu DnaE was constructed using five synthetic oligonucleotides optimized for expression in S. cerevisiae (FIG. 23). The Npu DnaE gene was constructed in three steps: (1) Dimer Extension (2) Full Length Construction (3) Full length amplification. In Step 1, ˜1 μg (20 μM) of oligonucleotides npu1+npu2, npu3+npu4, and npu5+npuVR (FIG. 23), that have overlapping regions, were mixed together in separate PCR tubes with 60 mM Tris-SO₄ (pH 8.9), 18 mM NH₄SO₄, 2 mM MgSO₄, 10 mM dNTPs, and 1.0 Unit Platinum High Fidelity Taq (Invitrogen). These dimers were extended using a 5 minute denaturation step at 95° C. followed by five rounds of incubation using the following cycle: 95° C. for 30 seconds, 55° C. for 30 seconds, and 72° C. for 15 seconds. In Step 2, full length Npu DnaE gene was constructed by mixing the dimers formed in Step 1 in a single reaction tube with 60 mM Tris-SO₄ (pH 8.9), 18 mM NH₄SO₄, 2 mM MgSO₄, 10 mM dNTPs, and 1.0 Unit Platinum High Fidelity Tag (Invitrogen) under the exact same conditions as in the dimer extension. Finally in Step 3, the full length gene was selectively amplified from the pool of incomplete dimer extensions to result in the full length gene, 1:10 of product from step (2) was mixed with npuVF, npuVR (FIG. 23), 60 mM Tris-SO₄ (pH 8.9), 18 mM NH₄SO₄, 2 mM MgSO₄, 10 mM dNTPs, and 1.0 Unit Platinum High Fidelity Taq (Invitrogen). The PCR reaction was initially denatured for 5 minutes at 95° C., followed by 25 cycles of 95° C. for 30 seconds, 55° C. for 30 seconds, and 72° C. for 30 seconds. The synthetic Npu DnaE gene was cloned into pIN01 digested with RsrII and XhoI (above) in the yeast strain EY93 by homologous recombination using lithium acetate transformations. This transformation resulted in the vector pIL100.

Next The Kan^{R} gene was then cloned into pIL100 at the Amp^{R} gene site. pIL100 was digested with ScaI in NEBuffer 3 [100 mM NaCl, 50 mM Tris-HCl pH 7.9, 10 mM MgCl₂, 1 mM Dithiothreitol] overnight at 37° C. The Kan^{R} gene was prepared by PCR amplification using S, T (FIG. 23), 60 mM Tris-SO₄ (pH 8.9), 18 mM NH₄SO₄, 2 mM MgSO₄, 10 mM dNTPs, and 1.0 Unit Platinum High Fidelity Taq (Invitrogen), with an initial denaturation of 5 minutes at 95° C. followed by 25 cycles of 95° C. for 30 seconds, 55° C. for 30 seconds, 72° C. for 1 minute. The Kan^{R} gene was cloned into pIL100 using in vivo homologous recombination and lithium acetate transformations. Positive clones were rechecked by PCR analysis, confirmation of growth on LB Kanamycin media, and no growth on LB Ampicillin. Successful clones were verified by sequencing, resulting in the completed pIL500 vector.

Construction of the Mixed Intein Libraries

Three additional pIL500 Lariat Libraries were constructed (pIL-XX): A random five amino acid library (Lib1), a random 10 amino acid library (Lib2), and a random five amino acid focused library (BNT codons, B=G,C,T , N=A,G,C,T) (Lib3). We replaced the Ser-Arg linker peptide that connects the Ssp DnaE I_{c} domain and the Npu DnaE I_{N} domain in pIL500 with a combinatorial five or ten amino acid peptide using a library oligonucleotide Lib1, Lib2 or Lib3 (FIG. 23). We PCR amplified the library oligonucleotide using primers L and npuLR (FIG. 23). We used the reaction conditions described above with seven amplification cycles consisting of a denaturing step at 95° C. for 30 seconds, an annealing step at 55° C. for 30 seconds, and an extension step at 72° C. for 15 seconds. We digested pIL500 with NruI (New England Biolabs, Ipswich, Mass.) and dephosphorylated the digested plasmid with 10 units of shrimp alkaline phosphatase (Fermentas). We cloned the library into pIL500 using in vivo homologous recombination (Ma, H. et al. (1987) Gene 58: 201-216) in EY93. We performed 100 lithium acetate transformations (Schiestl, R. H. & Gietz, R. D. (1989) Curr. Genet. 16: 339-346) with each transformation containing 400 ng of amplified library and 1 μg of NruI-digested pIIL500.

Mutant Lariat Inteins with Enhanced Stability

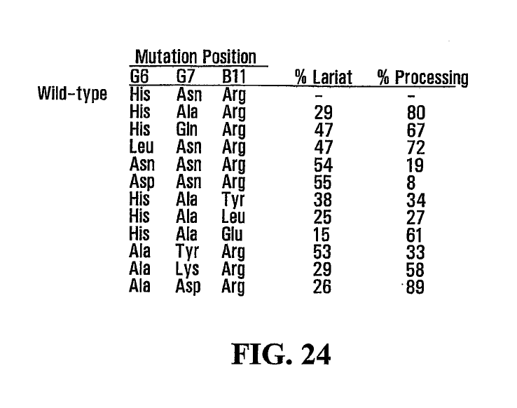

Stabilization of lactone-cyclized lariat: The lariat peptide is generated by inhibiting Asn-cyclization in the intein-cyclization reaction, which produces a peptide that is cyclized through a lactone bond. We generated a lariat by mutating Asn at position I_{C}., (G7) to Ala in the lariat intein construct. The lactone-bond cyclizing the lariat is more susceptible to hydrolysis than an amide bond and we have shown that ˜50% of the lariat exists in the lactone-cyclized state when expressed in E. coil. To improve our lariat yeast two-hybrid assay, to make it easier to purify and store lariats, and to expand the applications using lariats, we tested whether mutant lariats could be generated that stabilize in the lariat. Based on the intein reaction mechanism, Intein crystal structures, and the ability of specific mutations to stabilize the branched intermediate in the normal intein reaction, which is analogous to the lactone-cyclized lariat, we identified specific mutations or combination of mutations in the lariat construct that should stabilize the lactone bond. We tested a small subset of mutations to confirm whether the lariat lactone bond can be stabilized further beyond what is observed in the Asn to Ala mutation at position I_{C-1} (G7) by introducing the following mutations into the lariat construct (Summarized in FIG. 24):

(i) Mutation of Asn at I_{C-1} (G7): Asn at position I_{C-1} (G7) is essential for Asn-cyclization in the intein-mediated cyclization reaction. The Asn side chain undergoes cyclization to cleave the I_{N} domain from lariat and produce a lactone-cyclized peptide. In the normal intein reaction, branched intermediate accumulates when Asn at position I_{C-1} (G7) is mutated to Lys (Kawasaki M, et al. (1997) J. Biol. Chem. 272:15668-15674). However, not all mutations at position I_{C-1} (G7) cause accumulation of branched intermediates. For example, mutation of Asn at position I_{C-1} (G7) to Ser or Ala (Chong, S, et al. (1996) J. Biol. Chem. 271:22159-22168) does not result in accumulation of branched intermediate. Interestingly, mutation of Asn at position I_{C-1} (G7) to Ala leads to the accumulation of branched intermediate if Cys, at position I_{C+1} (G8), is also mutated to Ser (Chong, S, et al. (1996) J. Biol. Chem. 271:22159-22168). Based on these observations, the environment surrounding the ester bond appears to play a role in stabilizing the branched intermediate. To demonstrate that mutations at position I_{C-1} (G7) besides the Asn to Ala mutation can enhance the stability of the lactone bond, we mutated Asn to Gln at position I_{C-1} (G7). This mutation resulted in the further stabilization of the lactone bond from the 29% lactone observed with Ala at I_{C-1} (G7) to 47% lactone observed with Gln at I_{C-1} (G7). Gln at position I_{C-1} (G7) still maintained good lariat processing (67%) (Flg. 24). This result is surprising as one would expect based on the results with other inteins that substitution of Asn at I_{C-1} (G7) with Gln would result in a functional intein that process all the way to a cyclic peptide. In alternative embodiments, amino acids having other bulky side chains that possess an alkyl gamma carbon may be used stabilize the lactone (for example by blocking water from accessing the lactone bond). The following amino acids may accordingly be substituted at position G7 (presented in order of preference for blocking water access to the lactone bond): Trp, Phe, Leu, Ile, Tyr, Met, Val, Arg, Lys, His, Glu, Asp

(ii) Mutation of His at I_{C-2} (G6): His at position I_{C-2} (G6) assists in Asn-cyclization by hydrogen bonding to the Asn carbonyl oxygen at position I_{C-1} (G7). Branched intermediate accumulates when His at position I_{C-2} (G6) is mutated to Leu, Asn, or Gln, which also depends on the amino acid at position I_{C-1} (G7), since when Asn at this position is mutated to Ala no branched intermediate is observed (Xu, M. & Perler, F. B. (1996) EMBO J. 15:5146-5153). This observation suggests that Asn at position I_{C-1} (G7) is important for branched intermediate accumulation caused by I_{C-2} (G6) mutations. To demonstrate that mutations at position I_{C-2} (G6) enhance lactone-cyclized lariat stability, we mutated His at position I_{C-2} (G6) to Leu, Asn, or Asp and measured lactone bond stability. Leu, Asn, and Asp mutations enhanced lariat stability to the 47%, 54%, and 55%, respectively. The Leu mutation maintained good processing (72%), while the Asn and Asp mutations decreased processing to 19% and 8%, respectively. In alternative embodiments, amino acids having other hydrophobic side chains may also be used to stabilize lactone bond (for example by excluding water from the reactive site while still permitting processing). The following amino acids may also accordingly be substituted at position G6: Trp, Phe, Leu, Ile, Met, Tyr.

(iii) Mutation of Arg at B11: In the absence of the His at I_{C-2} (G6), it has been shown that Arg at position B11 can assist in Asn-cyclization by hydrogen bonding to the Asn carbonyl oxygen at position I_{C-1} (G7) (Ding, Y, et al. (2003) J. Biol. Chem. 278:39133-39142). B11 is predominately Lys or Arg, when I_{C-2} (G6) is not His. Currently, there are no mutagenic studies on the role of Arg at position B11 in accumulating branched intermediates. However, certain inteins can only function with specific amino acid at position I_{C-2} (G6) or B11. We have assessed the ability of single mutations at I_{C-2} (G6) or B11 to stabilize the lariat lactone. Mutation of lariat construct a B11 from Arg to Tyr increased the lariat stability to 38%, mutation to Leu had no effect on lariat stability, and mutation to Asp decreased lariat stability to 15%. Tyr, Leu, and Asp decreased lariat processing to 27%, 34%, and 61%, respectively. We also mutated His at position I_{C-2} (G6) to Ala combined with mutations a position I_{C-1} (G7). Mutation of I_{C-2} (G6) to Ala and I_{C-1} (G7) to Tyr increased lariat stability to 53%, whereas mutation of I_{C-1} (G7) to Asp or Lys has no effect on stability. Mutation of I_{C-2} (G6) to Ala and I_{C-1} (G7) to Tyr or Lys decreased processing to 33% and 58%, respectively, whereas mutation of I_{C-1} (G7) to Asp increased processing to 89%. In alternative embodiments, mutation of G6 (His) to Ala and mutation of B11 (Arg) to another large side chain may be used to stabilize the lactone bond (for example by excluding water while continuing to allow processing). The following amino acids may accordingly be substituted at B11 in conjunction with substituting Ala at G6: Lys, Tyr, Phe, Trp, His, Gln, Glu.

(iv) Position F4 (Asp): The amino acid at this position coordinates water near the lactone bond and participates in Steps 1 and 2 by polarizing the carbonyl to assist in nucleophilic attack by A1 and G8. Mutation of F4 from Asp to Glu, and Gln may accordingly be undertaken so as to allow Steps 1 and 2 to occur, while stabilizing the lactone bond (for example by excluding water from the region around the lactone bond).

(v) Position F13 (His): A His to Ala mutation at F13 does not block Step 3, while substitution of a bulky hydrophobic amino acid at F13 may be used to stabilize the lactone bond: including substitution of Phe, Leu , or Ile.

(vi) Position F14 (Asn): Bulky or charged amino acids substituted at F14 may be used to disrupt the correct positioning of F13 and thus block Asn cyclization and stabilize the lactone bond, including substitution of: Trp, Phe, Tyr, Leu, Lys, Arg

(vii) Position F15 (Phe): A mutation at F15 to Ala blocks Asn Cyclization, while mutation to Tyr slightly inhibits Asn cyclization. Accordingly, mutation of F15 to a bulky hydrophobic amino acid may be used to block Asn cyclization and exclude water around the lactone bond, thus stabilizing it. The following amino acids may accordingly be substituted at positioning of F13 to stabilize the lactone bond: Trp, Leu,

Methods

Mutations were constructed by site directed mutagenesis at the G6, G7, and B11 positions using Phusion™ Site-Directed Mutagenesis Kit (Finnzymes) as per manufacturers instructions. We purified the His-tag lariats using a Ni-NTA Spin Kit (Qiagen). Briefly, we transformed BL21-CP E. coil (Invitrogen) with the mutant intein expression plasmids. We expressed the mutant L2 lariats by inducing a 0.6 OD₆₀₀ culture of BL21-CP with 1 mM IPTG for four hours. We washed the cells, suspended them in phosphate buffered saline, 0.05% Triton X-100, 1 mg/mL lysozyme and lysed them using a FastPrep 120. We centrifuged the lysate at 10,000×g for 20 minutes at 4° C. and passed the clarified supernatant through a Ni-NA column (Qiagen). We washed the column 3 times with 50 mM NaH₂PO₄ and 300 mM NaCl and eluted the L2 lariat using 50 mM NaH₂PO₄ pH 7.0, 300 mM NaCl, and 250 mM Imidazole. We separated and desalted the His-tag purified lariats using a C4 reverse phase column (Symmetry300™ C4 3.5 μm 2.1×50 mm Column) (Waters, Milford, Mass.) with a gradient of 95% Buffer A/5% Buffer B to 25% Buffer A/75% Buffer B over 20 minutes (Buffer A: H₂O and 0.1% Formic acid (v/v), Buffer B: Acetonitrile and 0.08% Formic acid (v/v)). We determined the molecular weights of the eluted proteins using ESI(+)-TOF MS (MicroMass LCT, Waters). We resolved the multi-charged lariat spectrums using maximum entropy software (MaxEnt, Waters) to determine the ratio of hydrolyzed to unhydrolyzed lariat post HPLC/Mass spectrometry analysis.

Enhancing ScFv Stability by Cyclization

Certain protein domains and motifs, especially small motifs with little tertiary structure, may not be easily targeted by small cyclic peptides. These types of targets may be more effectively targeted by ScFvs, which are effective at binding small linear peptide epitopes. A common requirement for both medical and non-medical applications involving ScFvs is high stability. ScFvs comprise immunoglobulin variable domains of heavy and light chains that are held together by a short peptide linker (Bird, R. E., et al. (1988) Science 242:423-426). Many ScFvs generated from natural antibodies or isolated by in vitro selection fail to function effectively in their designed application as they often denature or aggregate (Worn, A. & Pluckthun, A. (2001) J. Mol. Biol. 305:989-1010). For intracellular applications, ScFvs are further destabilized by their inability to form a conserved intra-domain disulfide bond under the reducing conditions of the cytoplasm (Worn, A. & Pluckthun, A. (2001) J. Mol. Biol. 305:989-1010). A variety of strategies, including rational and evolutionary approaches, have been used to enhance the intra- and inter-domain stability of ScFvs (Worn, A. & Pluckthun, A. (2001) J. Mol. Biol. 305:989-1010) to produce stable scFv frameworks that a variety of complementarity determining regions (CDRs) regions can be grafted onto. These ScFv frameworks often work well, however in many cases specific CDRs are not compatible with given frameworks (Worn, A. & Pluckthun, A. (1998) FEBS Lett. 427:357-361). To create more universal and stable ScFv frameworks that are compatible with the yeast two-hybrid and other assays ScFvs can be cyclized or their surface charge can be increased, both of which should enhance stability and solubility.

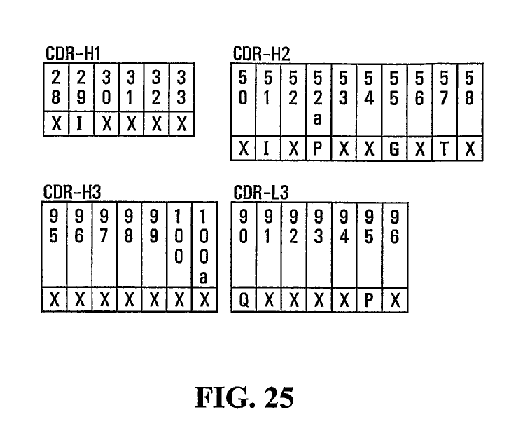

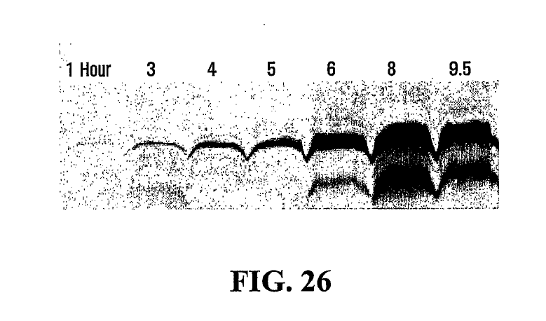

We constructed several ScFv libraries in yeast two-hybrid expression vectors using the ScFv framework used by Tanaka at al., for yeast two-hybrid assays (Tanaka, T., at al., (2003) Nucl. Acids Res. 31:e23). We randomized the three heavy chain variable loops and one light chain variable loop based on the design reported by Fellouse at al., for use in phage display (Fellouse, F. A. et al., (2004) Proc. Natl. Acad. Sci. USA 101:12467-12472; Fellouse, F. A., at al. (2005) J. Mol. Biol. 348:1153-1162). The residues chosen for randomization are shown in FIG. 25. Two libraries have three CDRs on the heavy chain and one CDR on the light chain randomized using combinations of Tyr and Ser, designated T4 or combinations of Tyr, Ala, Asp, and Ser designated K4. This limited amino acid diversity was chosen based on reports by Fellouse at al., where they showed that ScFvs with micromolar to nanomolar binding affinity could be isolated using ScFvs randomized with T4 (Fellouse, F. A., et al. (2005) J. Mol. Biol. 348:1153-1162) or K4 (Fellouse, F. A. et al., (2004) Proc. Natl. Acad. Sci. USA 101:12467-12472) diversity. Two additional libraries that are based on the T4 and K4 libraries have been generated by cloning these libraries in the lariat construct and they are designated cyc-T4 and cyc-K4. We have analyzed the expression of these libraries using Western analysis (FIG. 26).

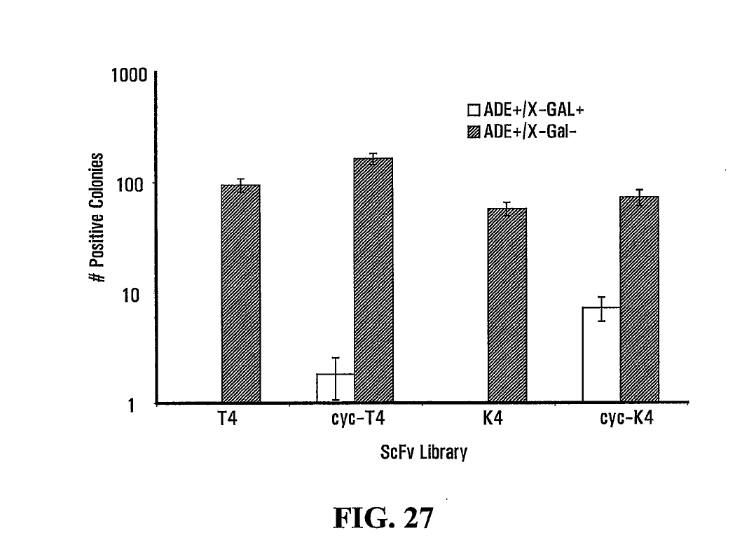

We calculated the effective library affinity, which is the number of positive interactions per library equivalent, using representative test proteins. We used two yeast two-hybrid reporter systems to evaluate effective library affinity. The first reporter is a “weak” Adenine reporter, which requires a lower affinity interaction to activate. The second reporter is a “stronger” adenine/LacZ reporter, which requires a higher affinity interaction to activate. For both the non-cyclized and lariat T4 and K4 libraries, we observed a small increase in the number of weak interacting library members (FIG. 27). Lariat cyclization of the T4 and K4 libraries increased the number of stronger interacting libraries members (FIG. 27).

An alternative method for stabilizing that can be used in conjunction with the lariat cyclization strategy involves enhancing or decreasing the ScFv surface charge. Recently, Lawrence at al., showed that radical changes in protein surface charge “supercharging” can significantly reduce aggregation tendency and improve the solubility of proteins without abolishing their function (Lawrence, M. A., at al., (2007) J. Am. Chem. Soc. 129:10110-10112). In some embodiments, supercharged cyclic ScFvs may accordingly be produced, for example with modifications that will decrease their propensity for aggregation (Worn, A. & Pluckthun, A. (2001) J. Mol. Biol. 305:989-1010). Crystal structures of ScFvs such as those reported by (Tanaka, T., at al., (2007) EMBO J. 26:3250-3259) can be used as guides for identifying surface residues to mutate. Surface residues on the ScFv that are solvent accessible can be identified using ASAView software (Ahmad, S., et al., (2004) BMC Bioinf. 5:51-56) or other similar software and techniques for identifying surface residues. Surface amino acids can be mutated to a positively (Lys, Arg, His) or negatively (Asp, Glu, Tyr, Cys) charged amino acids, depending on whether the desired charge on the ScFv is positive, negative, or a mixture of positive and negative charges.

ScFvs expressed as lariats, unprocessed, or dicysteine inteins that interact with a given target can be isolated from synthetic ScFv libraries, where the variable regions or CDRs are randomized with two or more amino acids, using genetic assays such as the yeast two-hybrid assay. Once they are isolated, ScFvs can be stably produced by expressing them as lariats or as head to tail cyclized ScFvs using the intein-mediated cyclic peptide/protein producing reaction. Alternatively, ScFvs can be cyclized by cross-linking. ScFvs can be engineered to contain small linker peptides at its N and C terminus that contain amino acids that can be can be cross-linked and give rise to a cyclized ScFv.

Cyclized and/or supercharged ScFvs for intracellular applications can also be constructed from existing monoclonal antibodies produced from hybridoma cell lines. In this case, the heavy and light chain antibody cDNA is used as a template to PCR amplify the light and heavy chain variable domains. These domains can then be cloned into one of the describe intein expression constructs, where they will be translated as a lariat, unprocessed, or discysteine ScFvs. Alternatively, the ScFv can be engineered to contain small linker peptides at its N and C terminus that contain amino acids that can be can be cross-linked and give rise to a cyclized ScFv.

ScFv and fragment antigen binding fragments (Fabs) that are isolated against a given target using an in vitro selection strategy such as phage display, yeast display, etc, can also be converted to an intracellular antibody by cyclizing and/or supercharging as described above. The fragment antigen binding (Fab fragment) is a region on an antibody, which binds to antigens. It is composed of one constant and one variable domain of each of the heavy and the light chain.

Cyclization or supercharging can be also applied to the expression of heavy or light chain fragments alone. In this case the heavy and light chain are used as affinity agents alone. It is also possible the cyclized and/or supercharged heavy and light chains can be expressed separately and that they will interact and form a functional Fv composed of both chains.

Cyclization or supercharging can be also applied to the expression of Fab fragments. Fabs are composed of one constant and one variable domain of each of the heavy and the light chain. Light and heavy chain regions of Fabs are held together by an inter-domain disulfide bond. In this case, Fabs can be stabilized in a reducing environment such as is present inside cells, by cyclization using one of the methods described above.

Purification of Cyclic ScFvs

We expect that ScFv cyclization and supercharging will reduce conformational breathing and hydrophobic aggregation and thus enhance stability and solubility. Cyclization has been shown to stabilize GFP (Iwai, H., et al., (2001) J. Biol. Chem. 276:16548-16554) and β-lactamase (Iwai, H. & Pluckthun, A. (1999) FEBS Lett. 459:166-172). We will cyclize ScFvs using intein-mediated cyclization and purify ScFvs using a protein L column. If an alternative method is required to purify higher levels of ScFvs, then we will use a histidine-tag as a linker to join the ScFv light and heavy chains, similar to the strategy used to purify cyclic GFP (Iwai, H., et al., (2001) J. Biol. Chem. 276:16548-16554) and β-lactamase (Iwai, H. & Pluckthun, A. (1999) FEBS Lett. 459:166-172).

Expression and Delivery of Cyclized Peptides and Proteins

In addition to being expressed intracellularly either transiently (plasmid transformation, adenovirus, etc) or by integration into the hosts genome (Stable cell lines, Retrovirus, etc), cyclized peptides, genomic fragments, and ScFvs, can be delivered exogenously for in vitro or in vivo applications. A variety of delivery systems are available for peptides using liposaccharides, lipopeptides, liposomes, and polyethylene glycol (PEG) conjugates (Reviewed by Ali, M & Manolios, N. (2002) Lett. Peptide Sci. 8:289-294). Peptides and proteins can also be delivered by conjugating them, either covalently or non-covalently to transduction peptides (Reviewed by Joliot, A & Prochiantz, A. (2004) Nature Cell Biol. 6:189-196).

Methods

Construction of ScFv and Library Design

ScFv Framework

A synthetic gene encoding the ScFv framework was constructed using codons optimized for S. cerevisiae expression. The amino acid sequences of the heavy and light chain were designed using the ScFv reported by Tanaka et a! (Tanaka, T., et al. (2003) Nucl. Acids Res. 31:e23). The region spanning the first and second CDR of the heavy chain was replaced with an NruI restriction endonuclease site to allow cloning of random amino acid libraries into CDR1 and 2 of the heavy chain. The heavy chain CDR3 was replaced by an XhoI restriction endonuclease site. The light and heavy chains were joined by a linker peptide consisting of glycine and serine repeats [G₄S]₃. The light chain CDRs were fixed based on anti-β-galactosidase ScFv reported by Martineau et al. (Martineau, P., et al. (1998) J. Mol. Biol. 280:117-127).

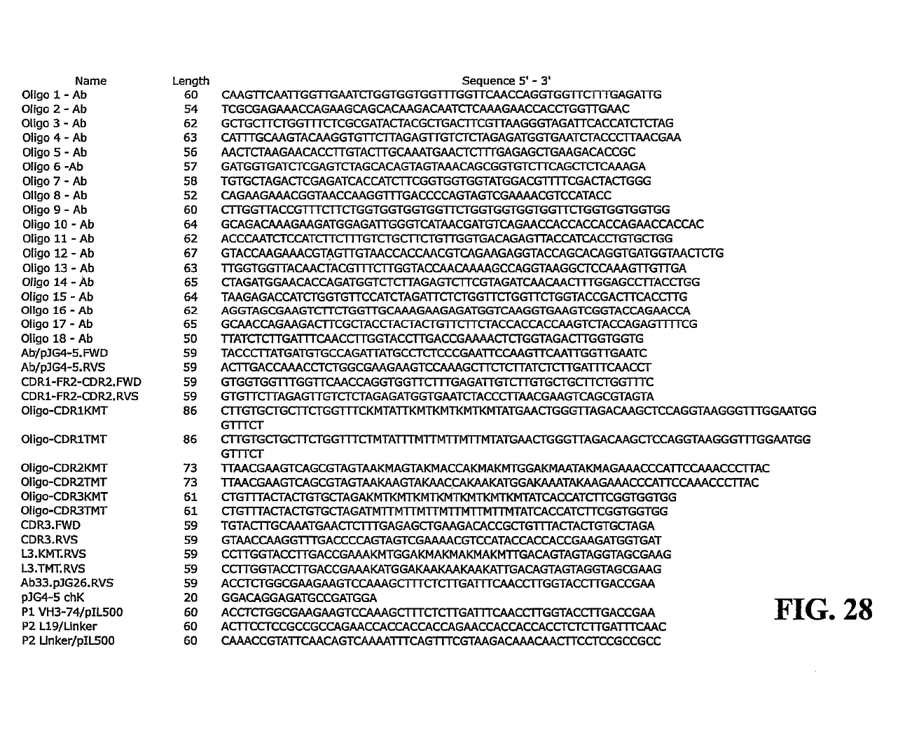

The program GeneDesign [http://slam.bs.jhmi.edu/gd/] was used to design eighteen overlapping oligonucleotides (Oligo 1-18-Ab) (FIG. 28) that were used to construct the synthetic ScFv intracellular antibody gene. The eighteen oligonucleotides (Oligo1-Ab to Oligo18-Ab) were mixed together (0.2 ng/μL of each) with HIFI Taq polymerase Buffer (60 mM Tris-SO₄ (pH 8.9), 18 mM (NH₄)₂SO4) (Invitrogen), 0.2 mM dNTPs, 2 mM MgSO₄, and 1.0 Unit Platinum HIFI Taq polymerase (Invitrogen). The reaction mix was incubated under the following conditions: 94° C. for 2 minutes, (94° C. for 30 seconds, 56° C. for 30 seconds, 68° C. for 1 minute (30 cycles), and 68° C. for 10 minutes. To amplify full-length gene product a second PCR was performed using 2 μL of PCR product from above and 0.2 μM Ab-pJG4-5.FWD primer and 0.2 μM of Ab-pJG4-5.RVS primer (FIG. 28) using the conditions described above.

Cloning ScFv Framework into Yeast Expression Vector

pIL500 was digested with 0.5 Units EcoRI endonuclease, 1 Units XhoI endonuclease, 1× y+/Tango Buffer (Fermentas) to remove the intein sequence. The reaction mixture was incubated at 37° C. overnight. The ScFv framework was cloned into EcoRI and XhoI digested pIL500 using homologous recombination. EcoRI and XhoI digested pIL500 and 40 μL of PCR amplified ScFv framework were transformed into yeast strain EY93 as described by Gietz et al. (Schiestl, R. H. & Gietz, R. D. (1989). Curr. Genet. 16:339-346) giving rise to the ScFv framework expression plasmid referred to as pScFv-Fr

CDR Library Oligonucleotides

The CDRs were randomized by cloning degenerate oligonucleotides flanked by fixed regions into the ScFv framework using homologous recombination. Combinatorial libraries consisting of Tyr (TAT codon) and Ser (TCT codon), referred to as T4 libraries, were constructed using TMT degenerate codons, where T=Thymine and M=Adenine or Cytosine. T4 libraries contain combinatorial Tyr and Ser CDRs in heavy chain CDRs 1-3 and light chain CDR3. Combinatorial libraries consisting of Tyr (TAT codon), Ser (TCT codon), Asp (GAT codon) and Ala (GCT codon), referred to as K4 libraries, were constructed using KMT degenerate codons, where M =Adenine or Cytosine and K=Thymine or Guanine. K4 libraries contain combinatorial Tyr, Ser, Ala, and Asp CDRs in heavy chain CDRs 1-3 and light chain CDR3. Oligonucleotides containing the degenerate CDRs (Oligo-CDR1KMT, Oligo-CDR1TMT, Oligo-CDR2KMT, Oligo-CDR2TMT, Oligo-CDR3KMT, Oligo-CDR3TMT, L3.KMT.RVS, and L3.KTMT.RVS are listed in FIG. 28.

Cloning Degenerate Heavy Chain CDR3 into ScFv Framework

To clone the degenerate heavy chain CDR3 into the ScFv framework, pScFv-Fr plasmid was digested with XhoI and gel purified. The degenerate CDR3 regions for the K or T libraries were cloned into pScFv-Fr using homologous recombination by transforming XhoI digested pScFv-Fr and PCR amplified Oligo-CDR3KMT into EY93 using lithium acetate transformation (Schiestl, R. H. & Gietz, R. D. (1989) Curr. Genet. 16:339-346), which gives the new plasmid pScFv-Fr-HCD3-K or pScFv-Fr-HCD3-T. Oligo-CDR3KMT was PCR amplified in the following reaction: 1× PCR Buffer, 0.2 mM dNTPs, 2 mM MgSO₄, 1 μM CDR.FWD, 1 μM CDR3.RVS, 0.02 μM Oligo-CDR3KMT or Oligo-CDR3KMT, 72 μL H₂O, 0.4 μL Taq polymerase. The PCR reaction was incubated under the following conditions: 95° C. for 1 minute, 95° C. for 30 seconds, 52 ‘C for 30 seconds, and 68° C. for 30 seconds (20 cycles).

Cloning Degenerate Heavy Chain CDR1 and 2 into ScFv Framework

To introduce CDR1 and CDR2 into the ScFv framework containing a degenerate CDR3, pScFv-Fr-HCD3-K and pScFv-Fr-HCD3-T was digested with NruI and gel purified. CDRs 1 and 2 were PCR amplified in the following reaction: 1× PCR Buffer, 0.2 mM dNTPs, 2 mM MgSO₄, 0.02 μM Oligo-CDR1TMT (or KMT), 0.02 μM Oligo-CDR2 TMT (or KMT), 0.2 μM CDR1-F2-CDR2.RVS, 0.2 μM CDR1-F2-CDR2.FWD, 80 μL H₂0, 0.4 μL Taq Polymerase. The PCR reaction was incubated under the following conditions: 95° C. for 2 minutes, 95° C. for 30 seconds, 56° C. for 30 seconds, 68° C. for 1 minute (25 cycles) and 68° C. for 10 minutes.

The degenerate K or T CDR1 and 2 regions were cloned into pScFv-Fr-HCD3-K and pScFv-Fr-HCD3-T, respectively using homologous recombination by transforming NruI digested pScFv-Fr-HCD3-K or pScFv-Fr-HCD3-T and PCR amplified CDR1 and 2 into EY93 using lithium acetate transformation (Schiestl, R. H. & Gietz, R. D. (1989) Curr. Genet. 16:339-346), which gives the new plasmid pScFv-Fr-HCD1-3-K and pScFv-Fr-HCD1-3-T.

Cloning Degenerate Light Chain CDR3 into ScFv Framework

To introduce light chain CDR3 into the ScFv framework containing degenerate heavy chain CDRs1-3, primers (FIG. 28) containing a degenerate light chain K or T library CDR3 was used to amplify the ScFv containing degenerate heavy chain CDRs1-3 from pScFv-Fr-HCD1-3-K and pScFv-Fr-HCD1-3-T. The following PCR reaction conditions were used 1× PCR Buffer (Invitrogen), 0.2 mM dNTPs, 1 μL pScFv-Fr-HCD1-3-K or pScFv-Fr-HCD1-3-T, 0.6 μM P1 pJG4-5 chK, 0.2 μM TMT (or KMT)L3.RVS, 0.2 μM Ab33.pJG26.RVS, 0.2 μM pJG4-5.RVS, 1 μL Taq polymerase. The reaction mixture was incubated under the following conditions: 95° C. for 2 minutes, 95° C. for 30 seconds, 55° C. for 30 seconds, 72° C. for 30 seconds (25 cycles), and 72° C. for 10 minutes. The PCR product was cloned into pIL500 digested with EcoRI and XhoI, giving rise to the plasmids expressing the K4 and T4 libraries, referred to as pScFv-K4 or pScFv-T4.

Cyclization of ScFv Library

pIL500 was digested with 10 Units of NruI and 1× NEBuffer in a 100 μL reaction. The reaction was incubated at 37° C. for 24 hours, DNA encoding ScFvs with T4 or K4 libraries were amplified from pScFv-K4 or pScFv-T4 using PCR using primer P1 VH3-74/pIL500 (FIG. 28), which contain overlapping complementary sequences to the IC domain. The second primer (P2 L19/Linker) (FIG. 28) contains DNA encoding a second linker peptide, which adds a peptide linker between the V_{H} domain and the I_{N} domain. ScFv libraries were PCR amplified using the following conditions: 1× PCR Buffer, 0.2 mM dNTPs, 1 μL pScFv-K4 or pScFv-T4, 0.2 μM P1 VH3-74/pIL500, 0.2 μM P2 L19/Linker, and 1 μL Taq polymerase. The PCR reaction was incubated under the following conditions: 95° C. for 2 minutes, 95° C. for 30 seconds, 50° C. for 30 seconds, 72° C. for 2 minute (30 cycles), and 72° C. for 7 minutes. A second PCR reaction was performed using a primer (P2 Linker/pIL500) that adds DNA that overlaps sequences to the I_{N} domain. The reaction was performed as described above. The PCR product was cloned into pIL500 digested with NruI giving rise to the plasmids expressing the K4 and 14 libraries, referred to as pScFv-cyc-K4 or pScFv-cyc-T4. 50 members from each library were sequenced to determine the percentage of functional ScFvs and to confirm library diversity.

Yeast Two-Hybrid Interaction Mating Screen

K4, cyc-K4, T4 and cyc-T4 libraries were screened against a pool of five baits: Bcr-Abl SH2 Domain, Bcr-Abl SH3 Domain, Bcr-Abl Coiled—coil domain, Bcr-Abl Y177 Motif, and Hck Tyr Kinase Domain. T4, Cyc-T4, K4, and cyc-K4 libraries were transformed into EY93 to give a final library diversity of 4.2×10⁸, 4.2×10⁶, 20×10⁶, and 2.2×10⁶, respectively. ScFv libraries and bait cells were cultured overnight in Trp-Glucose and His-Glucose media, respectively, to an optical density above 0.5. Cells were centrifuged at 4000 rpm for 5 minutes at room temperature and washed in 1× PBS. The cells were centrifuged again as above and re-suspended in YPD+Adenine (40 mg/L) media. Cells were mixed at a 60×10⁶ ScFv library cells to 30×10⁶ of each bait (Total baits 150×10⁶) ratio and plated on YPD+Adenine plates and incubated overnight at 30° C. Cells were scraped off the plate the next day, washed with 40 mL H₂0, re-suspended in glycerol freeze down solution (according to pellet size) and stored at −80 ‘C. The mating efficiency as calculated and the number of diploids determined.

6×10⁶ cells from each library (normalized for correctly cloned sequences) were plated on His-, Trp-, Leu-Galactose/Sucrose plates to score for ScFvs that interacted with the bait and activated the LEU2 yeast two-hybrid reporter gene. After one week the plates were replica plated to His-, Trp-, Ade-, X-Gal Galactose/Sucrose plates. Cells that grew were classified as weak interactors and cells that grew and turned blue were classified as strong interactors (FIG. 27). The assay was repeated five times.

Western Analysis of ScFv Lariat

An individual member of the cyc-K4 library member was grown up overnight in Trp-Glucose media. The cells were centrifuged at 4000 rpm for 5 minutes at room temperature, washed in 40 mL H₂0, centrifuged, and re-suspended in 10 mL Trp-Galactose/Raffinose media. 1 mL time points were taken at 1, 3, 4, 5, 6, 8, 9.5, and 25 hours to analyze expression of the cyc-K4 member. Aliquots at specific time points were centrifuged at 4000 rpm for 5 minutes at room temperature and washed in 1 mL of H₂O. The cells were re-suspended in 100 μL H₂O and 100 μL 0.2 M NaOH, lightly vortexed, and incubated for 5 minutes at room temperature. The reaction was centrifuged and re-suspended in 50 μL SOS-loading buffer (0.06 M Tris-HCl, pH 6.8, 5% glycerol, 2% SIDS, 4% β-mercapto-ethanol, 0.0025% bromophenol blue) and heated for 3 minutes at 95° C. The samples were analyzed using 15% SDS PAGE. The gel was electroblotted to a nitrocellulose membrane for 45 minutes at 15 V. The nitrocellulose membrane was incubated in 10 mL blocking buffer (Licor Biosciences) for one hour. The membrane was incubated in an a-HA primary antibody solution (50 μL of α-HA antibody (Santa Cruz), 10 mL blocking buffer, 5 μL Tween) overnight. The membrane was washed three times with 1× PBS incubated for one hour with a-mouse secondary antibody (Licor Biosciences). The membrane was washed 3 times with 1 X PBS and visualized using infrared Licor Analyzer.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A recombinant nucleic acid sequence encoding a split intein polypeptide, wherein the split intein polypeptide comprises, in amino to carboxy order:
an I_{C}domain comprising an F block and a G block, the F block being at least 80% identical to the sequence rVYDLpV**a--HNFh, designated respectively as positions F1 to F16, and the G block being at least 80% identical to the sequence NGhhhHNp, designated respectively as positions G1 to G8;
an extein domain attached to the C terminal portion of the G block; and,
an I_{N} domain attached to the C terminal portion of the extein domain, the I_{N} domain comprising an A block and a B block, the A block being at least 80% identical to the sequence Ch--Dp-hhh--G, designated respectively as positions A1 to A13, and the B block being at least 80% identical to the sequence G--h-hT--H-hhh, designated respectively as positions B1 to B14;
wherein:
a capital letter represents an amino acid designated by the single letter amino acid code;
“h” represents a hydrophobic residue selected from the group consisting of G, B, L, I, A and M;
“a” represents an acidic residue selected from the group consisting of D and E;
“r” represents an aromatic residue selected from the group consisting of F, Y and W;
“p” represents a polar residue selected from the group consisting of S, T and C;
“-” represents any amino acid; and
“*” represents optional gaps;
and wherein:
the residue encoded at position G7 is Q, W, F, L, I, Y, M, V, R, K, H, E or D; and/or
the residue encoded at position G6 is L, N, D, W, F, I, M or Y; and/or
the residue encoded at position B 11 is K, Y, F, W, H, Q or E; and/or
the residue encoded at position G6 is A and G7 is Y; and/or,
the residue encoded at position G6 is A and B 11 is K, Y, F, W, H, Q or E; and/or,
the residue encoded at position F4 is E or Q; and/or,
the residue encoded at position F13 is F, L or I; and/or,
the residue encoded at position F14 is W, F, Y, L, K or R; and/or
the residue encoded at position F15 is W or L; and/or,
the residue at position B9 is not R or T and is a non-catalytic amino acid for an N—X acyl shift; and/or,
the residue at position B 10 is not R or T and is a non-catalytic amino acid for an N—X acyl shift; and/or,
the residue at position F2 is not R or T and is a non-catalytic amino acid for an N—X acyl shift; and/or,
the residue at position F6 is not S, T or C and is a non-catalytic amino acid for a transesterification reaction involving a nucleophilic amino acid at position G8 attacking an ester or thioester bond.

2. The recombinant nucleic acid of claim 1, wherein:
the residue encoded at position G7 is Q; or
the residue encoded at position G6 is L, N or D; or
the residue encoded at position B11 is Y; or
the residue encoded at position G6 is A and G7 is Y.

3. The recombinant nucleic acid of claim 1, wherein the extein domain comprises an immunoglobulin encoding region that encodes an immunoglobulin molecule comprised of a heavy chain variable region attached by linkers to a light chain variable region, a first linker attaching the C-terminal region of the heavy chain variable region to the N-terminal region of the light chain variable region and a second linker attaching the N-terminal region of the heavy chain variable region to the C-terminal region of the light chain variable region, wherein the linkers comprise a polypeptide chain of at least 10 amino acids, wherein:
the heavy chain variable region comprises one or more heavy chain framework regions selected from the group consisting of HFR1, HFR2, HFR3, and HFR4; and the heavy chain variable region further comprises one or more complementarity determining regions selected from the group consisting of CDR-H1, CDR-H2, CDR-H3; with the heavy chain framework and complementarity determining regions arranged in accordance with the formula HFR1--CDR-H1--HFR2--CDR-H2--HFR3--CDR-H3--HFR4; and,
the light chain variable region comprises and one or more light chain framework regions selected from the group consisting of LFR1, LFR2, LFR3 and LFR4; and the light chain variable region further comprises one or more complementarity determining regions selected from the group consisting of CDR-L1, CDR-L2 and CDR-L3; with the light chain framework and complementarity determining regions arranged in accordance with the formula LFR1--CDR-L1--LFR27-CDR-L2--LFR3--CDR-L3--LFR4;
and wherein,
(i) HFR1 is a first heavy chain framework region consisting of a sequence of about 30 amino acid residues;
(ii) HFR2 is a second heavy chain framework region consisting of a sequence of about 14 amino acid residues;
(iii) HFR3 is a third heavy chain framework region consisting of a sequence of about 29 to about 32 amino acid residues;
(iv) HFR4 is a fourth heavy chain framework region consisting of a sequence of 7 to about 9 amino acid residues;
(v) CDR-H1 is a first heavy chain complementary determining region;
(vi) CDR-H2 is a second heavy chain complementary determining region;
(vii) CDR-H3 is a third heavy chain complementary determining region;
(viii) LFR1 is a first light chain framework region consisting of a sequence of about 22 to about 23 amino acid residues;
(ix) LFR2 is a second light chain framework region consisting of a sequence of about 13 to about 16 amino acid residues;
(x) LFR3 is a third light chain framework region consisting of a sequence of about 32 amino acid residues;
(xi) LFR4 is a fourth light chain framework region consisting of a sequence of about 12 to about 13 amino acid residues;
(xii) CDR-L1 is a first light chain complementary determining region;
(xiii) CDR-L2 is a second light chain complementary determining region; and,
(xiv) CDR-L3 is a third light chain complementary determining region.

4. A host cell comprising the recombinant nucleic acid of claim 1, wherein the split intein polypeptide is processed in the host cell in a self catalyzed reaction to form at least one cyclized polypeptide having no more than one linear terminal end.

5. A host cell comprising the recombinant nucleic acid of claim 3, wherein the split intein polypeptide is processed in the host cell in a self catalyzed reaction to form an immunoglobulin molecule having no more than one linear terminal end and having the conformation of an immunoglobulin fold.

6. The host cell of claim 4, wherein the cyclized polypeptide has one linear terminal end, being a C-terminal end or an N-terminal end.

7. The host cell of claim 6, wherein the cyclized polypeptide forms a lariat peptide.

8. The host cell of claim 5, wherein the immunoglobulin molecule forms a lariat peptide.

9. The host cell of claim 7, wherein the lariat peptide comprises a lactone junction.

10. The host cell of claim 6, wherein the cyclized polypeptide is cyclic and has no linear terminal end.

11. The host cell of claim 5, wherein the immunoglobulin molecule is cyclic and has no linear terminal end.

12. A host cell adapted for assaying interactions between fusion proteins, the cell comprising:
a first recombinant gene coding for a prey fusion protein, the prey fusion protein comprising a transcriptional repressor or activator domain and a first heterologous amino acid sequence;
a second recombinant gene coding for a bait fusion protein, the bait fusion protein comprising a DNA-binding domain and a second heterologous amino acid sequence; and,
a recombinant reporter gene coding for a detectable gene product, the recombinant reporter gene comprising an operator DNA sequence capable of binding to the DNA binding domain of the bait fusion protein;
wherein expression of the reporter gene is modulated in response to binding between the first heterologous amino acid sequence and the second heterologous amino acid sequence; and,
wherein at least one of the recombinant genes comprises the nucleic acid of claim 1.

13. A method of assaying for interactions between fusion proteins in cells, the method comprising:
causing the cells to express a recombinant gene coding for a prey fusion protein, the prey fusion protein comprising a transcriptional repressor or activator domain and a first heterologous amino acid sequence;
causing the cells to express a recombinant gene coding for a bait fusion protein, the bait fusion protein comprising a DNA-binding domain and a second heterologous amino acid sequence;
wherein at least one of the recombinant genes comprise the nucleic acid of claim 1;
providing the cells with a recombinant reporter gene coding for a detectable gene product, the recombinant reporter gene comprising an operator DNA sequence capable of binding to the DNA-binding domain of the bait fusion protein, wherein expression of the reporter gene is modulated in response to binding between the first heterologous amino acid sequence and the second heterologous amino acid sequence; and,
assaying for expression of the detectable gene product.

14. An immunoglobulin molecule having no more than one linear terminal end and having the conformation of an immunoglobulin fold comprised of a heavy chain variable region attached by linkers to a light chain variable region, a first linker attaching the C-terminal region of the heavy chain variable region to the N-terminal region of the light chain variable region and a second linker attaching the N-terminal region of the heavy chain variable region to the C-terminal region of the light chain variable region, wherein the linkers comprise flexible covalent molecular links of at least approximately 50 Angstroms in length, wherein:
the heavy chain variable region comprises one or more heavy chain framework regions selected from the group consisting of HFR1, HFR2, HFR3, and HFR4; and the heavy chain variable region further comprises one or more complementarity determining regions selected from the group consisting of CDR-H1, CDR-H2, CDR-H3; with the heavy chain framework and complementarity determining regions arranged in accordance with the formula HFR1--CDR-H1--HFR2--CDR-H2--HFR3--CDR-H3--HFR4; and,
the light chain variable region comprises and one or more light chain framework regions selected from the group consisting of LFR1, LFR2, LFR3 and LFR4; and the light chain variable region further comprises one or more complementarity determining regions selected from the group consisting of CDR-L1, CDR-L2 and CDR-L3; with the light chain framework and complementarity determining regions arranged in accordance with the formula LFR1--CDR-L1--LFR27-CDR-L2--LFR3--CDR-L3--LFR4;
and wherein,
(i) HFR1 is a first heavy chain framework region consisting of a sequence of about 30 amino acid residues;
(ii) HFR2 is a second heavy chain framework region consisting of a sequence of about 14 amino acid residues;
(iii) HFR3 is a third heavy chain framework region consisting of a sequence of about 29 to about 32 amino acid residues;
(iv) HFR4 is a fourth heavy chain framework region consisting of a sequence of 7 to about 9 amino acid residues;
(v) CDR-H1 is a first heavy chain complementary determining region;
(vi) CDR-H2 is a second heavy chain complementary determining region;
(vii) CDR-H3 is a third heavy chain complementary determining region;
(viii) LFR1 is a first light chain framework region consisting of a sequence of about 22 to about 23 amino acid residues;
(ix) LFR2 is a second light chain framework region consisting of a sequence of about 13 to about 16 amino acid residues;
(x) LFR3 is a third light chain framework region consisting of a sequence of about 32 amino acid residues;
(xi) LFR4 is a fourth light chain framework region consisting of a sequence of about 12 to about 13 amino acid residues;
(xii) CDR-L1 is a first light chain complementary determining region;
(xiii) CDR-L2 is a second light chain complementary determining region; and,
(xiv) CDR-L3 is a third light chain complementary determining region.

15. The immunoglobulin molecule of claim 14, wherein the linkers are polypeptide linkers comprising 14 to 25 amino acids.

16. The immunoglobulin molecule of claim 15, wherein the polypeptide linkers are comprised of glycine and serine amino acids.

17. A host cell comprising the recombinant nucleic acid of claim 2, wherein the split intein polypeptide is processed in the host cell in a self catalyzed reaction to form at least one cyclized polypeptide having no more than one linear terminal end.

18. The host cell of claim 8, wherein the lariat peptide comprises a lactone junction.


SEQUENCE LISTING (98 sequences)

SEQ ID NO: 1 - protein, 16 aa, Artificial Sequence
  Synthetic Peptide Sequence
XVYDLXVXXXXXHNFX

SEQ ID NO: 2 - protein, 8 aa, Artificial Sequence
  Synthetic Peptide Sequence
NGXXXHNX

SEQ ID NO: 3 - protein, 13 aa, Artificial Sequence
  Synthetic Peptide Sequence
CXXXDXXXXXXXG

SEQ ID NO: 4 - protein, 14 aa, Artificial Sequence
  Synthetic Peptide Sequence
GXXXXXTXXHXXXX

SEQ ID NO: 5 - protein, 8 aa, Artificial Sequence
  Synthetic Peptide Sequence
NGXXXXXX

SEQ ID NO: 6 - protein, 14 aa, Artificial Sequence
  Synthetic Peptide Sequence
GXXXXXTXXXXXXX

SEQ ID NO: 7 - protein, 16 aa, Artificial Sequence
  Synthetic Peptide Sequence
XXYXLXVXXXXXXXXX

SEQ ID NO: 8 - protein, 8 aa, Artificial Sequence
  Synthetic Peptide Sequence
SWDLPGEY

SEQ ID NO: 9 - protein, 24 aa, Artificial Sequence
  Synthetic Peptide Sequence
IFDIGLPQDHNFLLANGAIAHASR

SEQ ID NO: 10 - protein, 10 aa, Artificial Sequence
  Synthetic Peptide Sequence
SXXXXXXXEY

SEQ ID NO: 11 - protein, 9 aa, Artificial Sequence
  Synthetic Peptide Sequence
RSWDLPGEY

SEQ ID NO: 12 - protein, 23 aa, Artificial Sequence
  Synthetic Peptide Sequence
GRKKRRQRRRPPQSRSWDLPGEY

SEQ ID NO: 13 - protein, 10 aa, Artificial Sequence
  Synthetic Peptide Sequence
SRSWDLPGEY

SEQ ID NO: 14 - protein, 10 aa, Artificial Sequence
  Synthetic Peptide Sequence
SSWDLPQGEY

SEQ ID NO: 15 - protein, 13 aa, Artificial Sequence
  Synthetic Peptide Sequence
HASXXXXXXXEYC

SEQ ID NO: 16 - protein, 10 aa, Artificial Sequence
  Synthetic Peptide Sequence
HASCPGCEYA

SEQ ID NO: 17 - protein, 13 aa, Artificial Sequence
  Synthetic Peptide Sequence
HASYYGLGNAEYC

SEQ ID NO: 18 - protein, 13 aa, Artificial Sequence
  Synthetic Peptide Sequence
HASERARHTSEYC

SEQ ID NO: 19 - protein, 13 aa, Artificial Sequence
  Synthetic Peptide Sequence
HASMKYVAMSEYC

SEQ ID NO: 20 - protein, 13 aa, Artificial Sequence
  Synthetic Peptide Sequence
HASEVTHIRNEYC

SEQ ID NO: 21 - protein, 13 aa, Artificial Sequence
  Synthetic Peptide Sequence
HASSDAVHSLEYC

SEQ ID NO: 22 - protein, 13 aa, Artificial Sequence
  Synthetic Peptide Sequence
HASNLANYGPEYC

SEQ ID NO: 23 - protein, 7 aa, Artificial Sequence
  Synthetic Peptide Sequence
HASRVTI

SEQ ID NO: 24 - protein, 5 aa, Artificial Sequence
  Synthetic Peptide Sequence
SLEYC

SEQ ID NO: 25 - protein, 4 aa, Artificial Sequence
  Synthetic Peptide Sequence
HASP

SEQ ID NO: 26 - protein, 8 aa, Artificial Sequence
  Synthetic Peptide Sequence
TPANLEYC

SEQ ID NO: 27 - protein, 7 aa, Artificial Sequence
  Synthetic Peptide Sequence
HASQCRW

SEQ ID NO: 28 - protein, 5 aa, Artificial Sequence
  Synthetic Peptide Sequence
YEEYC

SEQ ID NO: 29 - protein, 6 aa, Artificial Sequence
  Synthetic Peptide Sequence
HASHNR

SEQ ID NO: 30 - protein, 6 aa, Artificial Sequence
  Synthetic Peptide Sequence
EEKEYC

SEQ ID NO: 31 - DNA, 80 bp, Artificial Sequence
  Synthetic Oligonucleotide
atggttaaggttattggtagaagatctttgggtgttcaaagaattttcgatattggtttgccacaagatcacaacttctt

SEQ ID NO: 32 - DNA, 80 bp, Artificial Sequence
  Synthetic Oligonucleotide
aaagacaaagcgtattcacacggaccgcacgacgagtgagcaatagcaccgttagccaacaagaagttgtgatcttgtgg

SEQ ID NO: 33 - DNA, 80 bp, Artificial Sequence
  Synthetic Oligonucleotide
tgtgaatacgctttgtctttcggtactgaaattttgactgttgaatacggtccattgccaattggtaagattgtttctga

SEQ ID NO: 34 - DNA, 80 bp, Artificial Sequence
  Synthetic Oligonucleotide
atagcttgagtgtaaactctaccttctggatcaacagagtaaacagaacagttaatttcttcagaaacaatcttaccaat

SEQ ID NO: 35 - DNA, 80 bp, Artificial Sequence
  Synthetic Oligonucleotide
agagtttacactcaagctattgctcaatggcacgatagaggtgaacaagaagttttggaatacgaattggaagatggttc

SEQ ID NO: 36 - DNA, 80 bp, Artificial Sequence
  Synthetic Oligonucleotide
atagccaacaattggtaatcagtagtcaagaatctgtgatcagaagtagctctaataacagaaccatcttccaattcgta

SEQ ID NO: 37 - DNA, 80 bp, Artificial Sequence
  Synthetic Oligonucleotide
gattaccaattgttggctattgaagaaattttcgctagacaattggatttgttgactttggaaaacattaagcaaactga

SEQ ID NO: 38 - DNA, 78 bp, Artificial Sequence
  Synthetic Oligonucleotide
cttaatagtaccagcatccaacaatgggaatggcaatctgtggttatccaaagcttcttcagtttgcttaatgttttc

SEQ ID NO: 39 - DNA, 59 bp, Artificial Sequence
  Synthetic Oligonucleotide
tacccttatgatgtgccagattatgcctctcccgaattcatggttaaggttattggtag

SEQ ID NO: 40 - DNA, 62 bp, Artificial Sequence
  Synthetic Oligonucleotide
tgaccaaacctctggcgaagaagtccaaagcttctcgagttacttaatagtaccagcatcca

SEQ ID NO: 41 - DNA, 69 bp, Artificial Sequence
  Synthetic Oligonucleotide
gtgctattgctcacgcttctnnknnknnknnknnknnknnkgaatactgtttgtctttcggtactgaat

SEQ ID NO: 42 - DNA, 59 bp, Artificial Sequence
  Synthetic Oligonucleotide
atattggtttgccacaagatcacaacttcttgttggctaacggtgctattgctcacgct

SEQ ID NO: 43 - DNA, 59 bp, Artificial Sequence
  Synthetic Oligonucleotide
atcttaccaattggcaatggaccgtattcaacagtcaaaatttcagtaccgaaagacaa

SEQ ID NO: 44 - DNA, 30 bp, Artificial Sequence
  Synthetic Oligonucleotide
aatttcagtaccgaaagacaaagcgtattc

SEQ ID NO: 45 - DNA, 30 bp, Artificial Sequence
  Synthetic Oligonucleotide
gaatacgctttgtctttcggtactgaaatt

SEQ ID NO: 46 - DNA, 29 bp, Artificial Sequence
  Synthetic Oligonucleotide
ccggaattcggttaaggttattggtagaa

SEQ ID NO: 47 - DNA, 30 bp, Artificial Sequence
  Synthetic Oligonucleotide
ttatctcgagttacttaatagtaccagcat

SEQ ID NO: 48 - DNA, 20 bp, Artificial Sequence
  Synthetic Oligonucleotide
ggacaggagatgccgatgga

SEQ ID NO: 49 - DNA, 59 bp, Artificial Sequence
  Synthetic Oligonucleotide
gtcgccgcatacactattctcagaatgacttggttgagtccggaattgccagctggggc

SEQ ID NO: 50 - DNA, 59 bp, Artificial Sequence
  Synthetic Oligonucleotide
tcatgccatccgtaagatgcttttctgtgactggtgagttcagaagaactcgtcaagaa

SEQ ID NO: 51 - DNA, 80 bp, Artificial Sequence
  Synthetic Oligonucleotide
tgtttgtcttacgaaactgaaattttgactgttgaatacggtttgttgccaattggtaagattgttgaaaagagaattga

SEQ ID NO: 52 - DNA, 80 bp, Artificial Sequence
  Synthetic Oligonucleotide
cattgagcaactggttgagtgtaaatgttaccgttgttatcaacagagtaaacagtacattcaattctcttttcaacaat

SEQ ID NO: 53 - DNA, 80 bp, Artificial Sequence
  Synthetic Oligonucleotide
actcaaccagttgctcaatggcacgatagaggtgaacaagaagttttcgaatactgtttggaagatggttctttgattag

SEQ ID NO: 54 - DNA, 80 bp, Artificial Sequence
  Synthetic Oligonucleotide
atttcatcaattggcaacatttgaccatcaacagtcatgaacttgtgatccttagtagctctaatcaaagaaccatcttc

SEQ ID NO: 55 - DNA, 66 bp, Artificial Sequence
  Synthetic Oligonucleotide
atgttgccaattgatgaaattttcgaaagagaattggatttgatgagagttgataacttgccaaac

SEQ ID NO: 56 - DNA, 59 bp, Artificial Sequence
  Synthetic Oligonucleotide
ttcttgttggctaacggtgctattgctcacgcttcgcgatgtttgtcttacgaaactga

SEQ ID NO: 57 - DNA, 59 bp, Artificial Sequence
  Synthetic Oligonucleotide
ccaaacctctggcgaagaagtccaaagcttctcgagttagtttggcaagttatcaactc

SEQ ID NO: 58 - DNA, 55 bp, Artificial Sequence
  Synthetic Oligonucleotide
gtgctattgctcacgcttctnnknnknnknnknnktgtttgtcttacgaaactga

SEQ ID NO: 59 - DNA, 70 bp, Artificial Sequence
  Synthetic Oligonucleotide
gtgctattgctcacgcttctnnknnknnknnknnknnknnknnknnknnktgtttgtcttacgaaactga

SEQ ID NO: 60 - DNA, 55 bp, Artificial Sequence
  Synthetic Oligonucleotide
gtgctattgctcacgcttctbntbntbntbntbnttgtttgtcttacgaaactga

SEQ ID NO: 61 - DNA, 59 bp, Artificial Sequence
  Synthetic Oligonucleotide
atcttaccaattggcaacaaaccgtattcaacagtcaaaatttcagtttcgtaagacaa

SEQ ID NO: 62 - DNA, 60 bp, Artificial Sequence
  Synthetic Oligonucleotide
caagttcaattggttgaatctggtggtggtttggttcaaccaggtggttctttgagattg

SEQ ID NO: 63 - DNA, 54 bp, Artificial Sequence
  Synthetic Oligonucleotide
tcgcgagaaaccagaagcagcacaagacaatctcaaagaaccacctggttgaac

SEQ ID NO: 64 - DNA, 62 bp, Artificial Sequence
  Synthetic Oligonucleotide
gctgcttctggtttctcgcgatactacgctgacttcgttaagggtagattcaccatctctag

SEQ ID NO: 65 - DNA, 63 bp, Artificial Sequence
  Synthetic Oligonucleotide
catttgcaagtacaaggtgttcttagagttgtctctagagatggtgaatctacccttaacgaa

SEQ ID NO: 66 - DNA, 56 bp, Artificial Sequence
  Synthetic Oligonucleotide
aactctaagaacaccttgtacttgcaaatgaactctttgagagctgaagacaccgc

SEQ ID NO: 67 - DNA, 57 bp, Artificial Sequence
  Synthetic Oligonucleotide
gatggtgatctcgagtctagcacagtagtaaacagcggtgtcttcagctctcaaaga

SEQ ID NO: 68 - DNA, 58 bp, Artificial Sequence
  Synthetic Oligonucleotide
tgtgctagactcgagatcaccatcttcggtggtggtatggacgttttcgactactggg

SEQ ID NO: 69 - DNA, 52 bp, Artificial Sequence
  Synthetic Oligonucleotide
cagaagaaacggtaaccaaggtttgaccccagtagtcgaaaacgtccatacc

SEQ ID NO: 70 - DNA, 60 bp, Artificial Sequence
  Synthetic Oligonucleotide
cttggttaccgtttcttctggtggtggtggttctggtggtggtggttctggtggtggtgg

SEQ ID NO: 71 - DNA, 64 bp, Artificial Sequence
  Synthetic Oligonucleotide
gcagacaaagaagatggagattgggtcataacgatgtcagaaccaccaccaccagaaccaccac

SEQ ID NO: 72 - DNA, 62 bp, Artificial Sequence
  Synthetic Oligonucleotide
acccaatctccatcttctttgtctgcttctgttggtgacagagttaccatcacctgtgctgg

SEQ ID NO: 73 - DNA, 67 bp, Artificial Sequence
  Synthetic Oligonucleotide
gtaccaagaaacgtagttgtaaccaccaacgtcagaagaggtaccagcacaggtgatggtaactctg

SEQ ID NO: 74 - DNA, 63 bp, Artificial Sequence
  Synthetic Oligonucleotide
ttggtggttacaactacgtttcttggtaccaacaaaagccaggtaaggctccaaagttgttga

SEQ ID NO: 75 - DNA, 65 bp, Artificial Sequence
  Synthetic Oligonucleotide
ctagatggaacaccagatggtctcttagagtcttcgtagatcaacaactttggagccttacctgg

SEQ ID NO: 76 - DNA, 64 bp, Artificial Sequence
  Synthetic Oligonucleotide
taagagaccatctggtgttccatctagattctctggttctggttctggtaccgacttcaccttg

SEQ ID NO: 77 - DNA, 62 bp, Artificial Sequence
  Synthetic Oligonucleotide
aggtagcgaagtcttctggttgcaaagaagagatggtcaaggtgaagtcggtaccagaacca

SEQ ID NO: 78 - DNA, 65 bp, Artificial Sequence
  Synthetic Oligonucleotide
gcaaccagaagacttcgctacctactactgttcttctaccaccaccaagtctaccagagttttcg

SEQ ID NO: 79 - DNA, 58 bp, Artificial Sequence
  Synthetic Oligonucleotide
ttatctcttgatttcaaccttggtaccttgaccgaaaactctggtagacttggtggtg

SEQ ID NO: 80 - DNA, 59 bp, Artificial Sequence
  Synthetic Oligonucleotide
tacccttatgatgtgccagattatgcctctcccgaattccaagttcaattggttgaatc

SEQ ID NO: 81 - DNA, 59 bp, Artificial Sequence
  Synthetic Oligonucleotide
acttgaccaaacctctggcgaagaagtccaaagcttctcttatctcttgatttcaacct

SEQ ID NO: 82 - DNA, 59 bp, Artificial Sequence
  Synthetic Oligonucleotide
gtggtggtttggttcaaccaggtggttctttgagattgtcttgtgctgcttctggtttc

SEQ ID NO: 83 - DNA, 59 bp, Artificial Sequence
  Synthetic Oligonucleotide
gtgttcttagagttgtctctagagatggtgaatctacccttaacgaagtcagcgtagta

SEQ ID NO: 84 - DNA, 86 bp, Artificial Sequence
  Synthetic Oligonucleotide
cttgtgctgcttctggtttckmtattkmtkmtkmtkmtatgaactgggttagacaagctccaggtaagggtttggaatgggtttct

SEQ ID NO: 85 - DNA, 86 bp, Artificial Sequence
  Synthetic Oligonucleotide
cttgtgctgcttctggtttctmtatttmttmttmttmtatgaactgggttagacaagctccaggtaagggtttggaatgggtttct

SEQ ID NO: 86 - DNA, 73 bp, Artificial Sequence
  Synthetic Oligonucleotide
ttaacgaagtcagcgtagtaakmagtakmaccakmakmtggakmaatakmagaaacccattccaaacccttac

SEQ ID NO: 87 - DNA, 73 bp, Artificial Sequence
  Synthetic Oligonucleotide
ttaacgaagtcagcgtagtaakaagtakaaccakaakatggakaaatakaagaaacccattccaaacccttac

SEQ ID NO: 88 - DNA, 61 bp, Artificial Sequence
  Synthetic Oligonucleotide
ctgtttactactgtgctagakmtkmtkmtkmtkmtkmtkmtatcaccatcttcggtggtgg

SEQ ID NO: 89 - DNA, 61 bp, Artificial Sequence
  Synthetic Oligonucleotide
ctgtttactactgtgctagatmttmttmttmttmttmttmtatcaccatcttcggtggtgg

SEQ ID NO: 90 - DNA, 59 bp, Artificial Sequence
  Synthetic Oligonucleotide
tgtacttgcaaatgaactctttgagagctgaagacaccgctgtttactactgtgctaga

SEQ ID NO: 91 - DNA, 59 bp, Artificial Sequence
  Synthetic Oligonucleotide
gtaaccaaggtttgaccccagtagtcgaaaacgtccataccaccaccgaagatggtgat

SEQ ID NO: 92 - DNA, 59 bp, Artificial Sequence
  Synthetic Oligonucleotide
ccttggtaccttgaccgaaakmtggakmakmakmakmttgacagtagtaggtagcgaag

SEQ ID NO: 93 - DNA, 59 bp, Artificial Sequence
  Synthetic Oligonucleotide
ccttggtaccttgaccgaaakatggakaakaakaakattgacagtagtaggtagcgaag

SEQ ID NO: 94 - DNA, 59 bp, Artificial Sequence
  Synthetic Oligonucleotide
acctctggcgaagaagtccaaagctttctcttgatttcaaccttggtaccttgaccgaa

SEQ ID NO: 95 - DNA, 20 bp, Artificial Sequence
  Synthetic Oligonucleotide
ggacaggagatgccgatgga

SEQ ID NO: 96 - DNA, 59 bp, Artificial Sequence; identical to SEQ ID NO: 94
  Synthetic Oligonucleotide

SEQ ID NO: 97 - DNA, 60 bp, Artificial Sequence
  Synthetic Oligonucleotide
acttcctccgccgccagaaccaccaccaccagaaccaccaccacctctcttgatttcaac

SEQ ID NO: 98 - DNA, 60 bp, Artificial Sequence
  Synthetic Oligonucleotide
caaaccgtattcaacagtcaaaatttcagtttcgtaagacaaacaacttcctccgccgcc